(19) United States
(12) Patent Application Publication
(10) Pub. No.: US 2022/0401559 A1
(43) Pub. Date: Dec. 22, 2022

(54) EXCIPIENT COMPOUNDS FOR PROTEIN FORMULATIONS

(71) Applicant: COMERA LIFE SCIENCES, INC., Woburn, MA (US)

(72) Inventors: David S. Soane, Coral Gables, FL (US); Philip Wuthrich, Lowell, MA (US); Robert P. Mahoney, Newbury, MA (US); Subhashchandra Naik, Watertown, MA (US); Timothy Tran, Medford, MA (US); Yuhong Zheng, Lexington, MA (US); Juan Davagnino, Durham, NC (US)

(21) Appl. No.: 17/716,248

(22) Filed: Apr. 8, 2022

Related U.S. Application Data
(63) Continuation of application No. PCT/US20/54749, filed on Oct. 8, 2020.
Application No. PCT/US20/54749 is a continuation-in-part of application No. 17/332,521, filed on May 27, 2021.
Application No. 17/332,521 is a continuation of application No. PCT/US2019/063374, filed on Nov. 26, 2019.
Application No. 17/332,521 is a continuation of application No. 17/011,014, filed on Sep. 3, 2020.
Application No. 17/011,014 is a continuation of application No. PCT/US2019/020751, filed on Mar. 5, 2019.
Application No. 17/011,014 is a continuation-in-part of application No. 15/896,374, filed on Feb. 14, 2018, now Pat. No. 11,357,857.
Application No. 15/896,374 is a continuation-in-part of application No. 15/331,197, filed on Oct. 21, 2016, now Pat. No. 10,478,498.
Application No. 15/331,197 is a continuation-in-part of application No. 14/966,549, filed on Dec. 11, 2015, now Pat. No. 9,605,051.
Application No. 14/966,549 is a continuation of application No. 14/744,847, filed on Jun. 19, 2015, now abandoned.
(60) Provisional application No. 62/912,375, filed on Oct. 8, 2019.
(60) Provisional application No. 62/773,018, filed on Nov. 29, 2018.
(60) Provisional application No. 62/639,950, filed on Mar. 7, 2018.
(60) Provisional application No. 62/679,647, filed on Jun. 1, 2018.
(60) Provisional application No. 62/459,893, filed on Feb. 16, 2017.
(60) Provisional application No. 62/014,784, filed on Jun. 20, 2014.
(60) Provisional application No. 62/083,623, filed on Nov. 24, 2014.
(60) Provisional application No. 62/136,763, filed on Mar. 23, 2015.
(60) Provisional application No. 62/245,513, filed on Oct. 23, 2015.
(60) Provisional application No. 62/245,513, filed on Oct. 23, 2015.

(51) Int. Cl. A61K 47/22; A61K 9/00; A61K 38/38; A61K 38/47; C12N 9/36; C12N 9/96; C07K 16/00; A61K 39/395; A61K 47/12; A61K 47/18; A61K 47/20; A61K 47/24; A61K 47/42; A61K 47/60

(52) U.S. Cl. CPC A61K 47/22; A61K 9/0019; A61K 38/385; A61K 38/47; C12N 9/2462; C12N 9/96; C07K 16/00; A61K 39/395; A61K 47/12; A61K 47/18; A61K 47/183; A61K 47/20; A61K 47/24; A61K 47/42; A61K 47/60; C12Y 302/01017; A61K 39/39591

(57) ABSTRACT

Disclosed herein are formulations with improved stability or reduced viscosity that comprise a therapeutic protein and a lyo-enhancing excipient, wherein the improved stability formulation is characterized by improved stability in comparison to a control formulation otherwise identical to the stability-enhanced formulation but lacking the lyo-enhancing excipient, and the reduced viscosity formulation is characterized by reduced viscosity in comparison to a control formulation otherwise identical to the reduced viscosity formulation but lacking the lyo-enhancing excipient. Further disclosed herein are methods of improving stability of therapeutic formulations, reducing viscosity of therapeutic formulations, or improving parameters of lyophilization processes.

### RELATED APPLICATIONS

This application is a continuation of International Application No. PCT/2020/054749, which designated the United States and was filed on Oct. 8, 2020, published in English, which claims the benefit of U.S. Provisional Application 62/912,375 filed Oct. 8, 2019. This application is also a continuation-in-part of U.S. application Ser. No. 17/332,521 filed May 27, 2021, which is a continuation of International Application No. PCT/2019/063374, which designated the United States and was filed on Nov. 26, 2019, published in English, which claims the benefit of U.S. Provisional Application 62/773,018 filed Nov. 29, 2018. U.S. application Ser. No. 17/332,521 is also a continuation-in-part of U.S. application Ser. No. 17/011,014 filed Sep. 3, 2020, which is a continuation of International Application No. PCT/US2019/020751 which designated the United States and was filed on Mar. 5, 2019, published in English, which claims the benefit of U.S. Provisional Application Ser. No. 62/639,950 filed Mar. 7, 2018 and U.S. Provisional Application Ser. No. 62/679,647 filed Jun. 1, 2018; U.S. application Ser. No. 17/011,014 is also a continuation-in-part of U.S. application Ser. No. 15/896,374 filed Feb. 14, 2018, which claims the benefit of U.S. Provisional Application No. 62/459,893 filed Feb. 16, 2017; U.S. application Ser. No. 15/896,374 is also a continuation-in-part of U.S. application Ser. No. 15/331,197 filed Oct. 21, 2016 (now U.S. Pat. No. 10,478,498), which is a continuation-in-part of U.S. application Ser. No. 14/966,549 filed Dec. 11, 2015 (now U.S. Pat. No. 9,605,051), which is a continuation of U.S. application Ser. No. 14/744,847 filed Jun. 19, 2015 (abandoned), which claims the benefit of U.S. Provisional Application No. 62/014,784 filed Jun. 20, 2014, U.S. Provisional Application No. 62/083,623, filed Nov. 24, 2014, and U.S. Provisional Application Ser. No. 62/136,763 filed Mar. 23, 2015; U.S. application Ser. No. 14/966,549 also claims the benefit of U.S. Provisional Application No. 62/245,513, filed Oct. 23, 2015; U.S. application Ser. No. 15/331,197 also claims the benefit of U.S. Provisional Application No. 62/245,513, filed Oct. 23, 2015. The entire contents of the above applications are incorporated by reference herein.

### FIELD OF APPLICATION

This application relates generally to biopolymer formulations, such as protein formulations, with stabilizing excipients.

### BACKGROUND

Biopolymers may be used for therapeutic or non-therapeutic purposes. Biopolymer-based therapeutics, such as formulations comprising proteins, antibodies, or enzymes, are widely used in treating disease. Non-therapeutic biopolymers, such as formulations comprising enzymes, peptides, or structural proteins, have utility in non-therapeutic applications such as household, nutrition, commercial, and industrial uses.

Of particular interest, for therapeutic and non-therapeutic uses are protein biopolymers. Proteins are complex biopolymers, each with a uniquely folded 3-D structure and surface energy map (hydrophobic/hydrophilic regions and charges). In concentrated protein solutions, these macromolecules may strongly interact and even inter-lock in complicated ways, depending on their exact shape and surface energy distribution. “Hot-spots” for strong specific interactions lead to protein clustering, increasing solution viscosity. To address these concerns, a number of excipient compounds are used in biotherapeutic formulations, aiming to reduce solution viscosity by impeding localized interactions and clustering. These efforts are individually tailored, often empirically, sometimes guided by in silico simulations. Combinations of excipient compounds may be provided, but optimizing such combinations again must progress empirically and on a case-by-case basis.

Biopolymers, such as proteins, used in therapeutic applications must be formulated to permit their introduction into the body for treatment of disease. For example, it is advantageous to deliver antibody and protein/peptide biopolymer formulations by subcutaneous (SC) or intramuscular (IM) routes under certain circumstances, instead of administering these formulations by intravenous (IV) injections. In order to achieve better patient compliance and comfort with SC or IM injection though, the liquid volume in the syringe is typically limited to 2 to 3 mL and the viscosity of the formulation is typically lower than about 20 centipoise (cP) so that the formulation can be delivered using conventional medical devices and small-bore needles. These delivery parameters do not always fit well with the dosage requirements for the formulations being delivered.

Antibodies, for example, may need to be delivered at high dose levels to exert their intended therapeutic effect. Using a restricted liquid volume to deliver a high dose level of an antibody formulation can require a high concentration of the antibody in the delivery vehicle, sometimes exceeding a level of 150 mg/mL. At this dosage level, the viscosity-versus-concentration plots of protein solutions lie beyond their linear-nonlinear transition, such that the viscosity of the formulation rises dramatically with increasing concentration. Increased viscosity, however, is not compatible with standard SC or IM delivery systems. The solutions of biopolymer-based or protein-based therapeutics are also prone to stability problems, such as precipitation, fragmentation, oxidation, deamidation, hazing, opalescence, denaturing, and gel formation, reversible or irreversible aggregation. The stability problems limit the shelf life of the solutions or require special handling.

One approach to producing protein formulations for injection is to transform the therapeutic protein solution into a powder that can be reconstituted to form a suspension suitable for IV, SC or IM delivery. Freeze-drying, spray drying and even precipitation followed by super-critical-fluid extraction are processes that have been used to generate protein powders for subsequent reconstitution. Lyophilization is a standard low-temperature technique to produce protein powders. An advantage of lyophilization is that the protein can be stored in a dry state, and this prevents degradation of the protein compared with storing the protein in a liquid solution. The lyophilized formulation can have less stringent requirements than liquid biologic formulations for cold chain delivery to patients due to the improved stability. Some proteins are not stable enough for storage in an aqueous buffer, and in these cases lyophilization can be the preferred formulation route.

Powdered protein suspensions in an aqueous or non-aqueous carrier fluid can be low in viscosity before re-dissolution (compared to solutions at the same overall dose) and thus may be suitable for SC or IM injection, provided the particles are sufficiently small to fit through the needle. However, protein particles that are present in the powder have the inherent risk of triggering immune response. The uncertain protein stability/activity following re-dissolution poses further concerns. While lyophilization can be an effective means to store proteins until they are used, the dried materials still have some disadvantages. For example, lyophilized proteins can still degrade in the powdered state, even under low temperature storage conditions. The degradation of the proteins can be a result of chemical degradation such as oxidation, proteolysis, deamidation, or hydrolysis; in other events the degradation can be a physical degradation such as aggregation, denaturation, or particle formation. The degradation of lyophilized protein might not be evident until the protein is dissolved in water or buffer solution for use. In some cases, the degraded protein can have poor solubility such that a hazy solution or particles can be seen after reconstitution of the lyophilized material. In view of these limitations, an alternative to lyophilization may be desirable, providing a stable formulation without the inherent limitations introduced by the lyophilization process itself.

If a lyophilized formulation is used, it is desirable for it to have high solubility, a fast reconstitution time, and low particle content after dissolution. As mentioned above, in some cases, a lyophilized protein can have limited solubility, leading to excessive time requirements to fully dissolve the material at the time of use. In some cases, a lyophilized protein can form highly viscous solutions after reconstitution, and this can also result in excessive time requirements to fully dissolve the material at the time of use. Another disadvantage of a highly viscous protein solution is that a higher amount of force would be required to dispense the liquid from a syringe. In some cases, the patient is expected to reconstitute the lyophilized protein in water or a buffer solution before self-administering the drug. Some patient populations have limited ability to sufficiently mix or self-administer lyophilized protein samples due to weakness, arthritis, low mechanical dexterity, or visual impairment, so it would be a benefit to have a lyophilized protein that dissolves more quickly in the solution and results in a low viscosity solution for administration. In some cases, a lyophilized protein can form highly viscous solutions after reconstitution, and this can also result in excessive time requirements to fully dissolve the material at the time of use.

Improving the lyophilization process and/or the formulations that it produces could allow the clinician to take advantage of the benefits of lyophilization (prolonged shelf life and stability, for example), while avoiding the drawbacks. Advantages of lyophilization for therapeutic proteins include the ease of processing the formulation liquid aseptically, preservation of protein activity due to relatively gentle processing conditions, enhanced stability of a dry powder for prolonged storage, and rapid, easy dissolution of the powder to form the reconstituted formulation. When these advantages are less prominent, there is a need either for alternatives to lyophilized formulations, or for improvements in the lyophilization process and/or the lyophilized formulation.

Therefore, under certain circumstances, there remains a need in the art for techniques to produce low viscosity protein formulations for therapeutic purposes that act as a substitute for lyophilized protein suspensions, offering a stable formulation while avoiding the limitations introduced by lyophilized and reconstituted protein powder suspensions. Under other circumstances, it would be advantageous to improve rather than supplant lyophilized formulations, for example by improving the lyophilization process, by improving stability of lyophilized formulations, or by decreasing viscosity of lyophilized formulations.

More complex antibody formulations, such as antibody-drug conjugates (ADCs), are especially vulnerable to viscosity and stability problems. An ADC links a small molecule drug to a monoclonal antibody (mAb) via a chemical linker; the mAb is targeted to a specific antigen on an abnormal “target cell,” and the small molecule drug is selected to have specific effects on that target cell. When the mAb contacts the target cell antigen, it and its attached drug is ingested by the cell and gains entry to the cell interior. Inside the cell, the mAb and/or the linker is broken down, releasing the drug to exert its biological effects on the cell. Typically, the drug is a chemotherapeutic agent that is too toxic to be released systemically. The ADC brings the chemotherapy into direct contact with the cancer cell that is its target. This attachment of a small molecule to a mAb can exacerbate the viscosity and stability problems that affect therapeutic protein formulations. The payload compound is typically a hydrophobic small molecule, which can exert significant effects on the stability, solubility, and solution interaction properties of the larger ADC as the drug-antibody ratio increases. High salt concentrations in the formulation can increase the hydrophobic interactions among ADC complexes, rendering the solubility of the ADC more sensitive to salt effects than an unconjugated antibody. Processing or storage of ADC solutions can incite aggregation or precipitation of the ADC species, especially at high drug-to-antibody ratios (DARs). Drug conjugation can also affect the conformational stability of the mAb, especially its Fc domain. In addition, drug conjugation may also reduce the net surface charge on the mAb, affecting the ADC's solubility.

In addition to the therapeutic applications of proteins described above, biopolymers such as enzymes, peptides, and structural proteins can be used in non-therapeutic applications. These non-therapeutic biopolymers can be produced from a number of different pathways, for example, derived from plant sources, animal sources, or produced from cell cultures.

The non-therapeutic proteins can be produced, transported, stored, and handled as a granular or powdered material or as a solution or dispersion, usually in water. The biopolymers for non-therapeutic applications can be globular or fibrous proteins, and certain forms of these materials can have limited solubility in water or exhibit high viscosity upon dissolution. These solution properties can present challenges to the formulation, handling, storage, pumping, and performance of the non-therapeutic materials, so there is a need for methods to reduce viscosity and improve solubility and stability of non-therapeutic protein solutions.

There remains a need in the art for a truly universal approach to reducing viscosity and/or improving stability in protein formulations, especially at high protein concentrations. There is an additional need in the art to achieve this viscosity reduction while preserving the activity of the protein. It would be further desirable to adapt the viscosity-reduction system to use with formulations having tunable and sustained release profiles, and to use with formulations adapted for depot injection. In addition, it is desirable to improve processes for producing proteins and other biopolymers.

### SUMMARY OF THE INVENTION

Disclosed herein, in embodiments, are liquid formulations comprising a protein and an excipient compound selected from the group consisting of hindered amines, anionic aromatics, functionalized amino acids, oligopeptides, short-chain organic acids, and low molecular weight aliphatic polyacids, wherein the excipient compound is added in a viscosity-reducing amount. In embodiments, the protein is a PEGylated protein and the excipient is a low molecular weight aliphatic polyacid. In embodiments, the formulation is a pharmaceutical composition, and the therapeutic formulation comprises a therapeutic protein, wherein the excipient compound is a pharmaceutically acceptable excipient compound. In embodiments, the formulation is a non-therapeutic formulation, and the non-therapeutic formulation comprises a non-therapeutic protein. In embodiments, the viscosity-reducing amount reduces viscosity of the formulation to a viscosity less than the viscosity of a control formulation. In embodiments, the viscosity of the formulation is at least about 10% less than the viscosity of the control formulation or is at least about 30% less than the viscosity of the control formulation, or is at least about 50% less than the viscosity of the control formulation, or is at least about 70% less than the viscosity of the control formulation, or is at least about 90% less than the viscosity of the control formulation. In embodiments, the viscosity is less than about 100 cP, or is less than about 50 cP, or is less than about 20 cP, or is less than about 10 cP. In embodiments, the excipient compound has a molecular weight of <5000 Da, or <1500 Da, or <500 Da. In embodiments, the formulation contains at least about 25 mg/mL of the protein, or at least about 100 mg/mL of the protein, or at least about 200 mg/mL of the protein, or at least about 300 mg/mL of the protein. In embodiments, the formulation comprises between about 5 mg/mL to about 300 mg/mL of the excipient compound or comprises between about 10 mg/mL to about 200 mg/mL of the excipient compound or comprises between about 20 mg/mL to about 100 mg/mL, or comprises between about 25 mg/mL to about 75 mg/mL of the excipient compound. In embodiments, the formulation has an improved stability when compared to the control formulation. In embodiments, the excipient compound is a hindered amine. In embodiments, the hindered amine is selected from the group consisting of caffeine, theophylline, tyramine, procaine, lidocaine, imidazole, aspartame, saccharin, and acesulfame potassium. In embodiments, the hindered amine is caffeine. In embodiments, the hindered amine is a local injectable anesthetic compound. The hindered amine can possess an independent pharmacological property, and the hindered amine can be present in the formulation in an amount that has an independent pharmacological effect. In embodiments the hindered amine can be present in the formulation in an amount that is less than a therapeutically effective amount. The independent pharmacological activity can be a local anesthetic activity. In embodiments, the hindered amine possessing the independent pharmacological activity is combined with a second excipient compound that further decreases the viscosity of the formulation. The second excipient compound can be selected from the group consisting of caffeine, theophylline, tyramine, procaine, lidocaine, imidazole, aspartame, saccharin, and acesulfame potassium. In embodiments, the formulation can comprise an additional agent selected from the group consisting of preservatives, surfactants, sugars, polysaccharides, arginine, proline, hyaluronidase, stabilizers, and buffers.

Further disclosed herein are methods of treating a disease or disorder in a mammal, comprising administering to said mammal a liquid therapeutic formulation, wherein the therapeutic formulation comprises a therapeutically effective amount of a therapeutic protein, and wherein the formulation further comprises an pharmaceutically acceptable excipient compound selected from the group consisting of hindered amines, anionic aromatics, functionalized amino acids, oligopeptides, short-chain organic acids, and low molecular weight aliphatic polyacids; and wherein the therapeutic formulation is effective for the treatment of the disease or disorder. In embodiments, the therapeutic protein is a PEGylated protein, and the excipient compound is a low molecular weight aliphatic polyacid. In embodiments, the excipient is a hindered amine. In embodiments, the hindered amine is a local anesthetic compound. In embodiments, the formulation is administered by subcutaneous injection, or an intramuscular injection, or an intravenous injection. In embodiments, the excipient compound is present in the therapeutic formulation in a viscosity-reducing amount, and the viscosity-reducing amount reduces viscosity of the therapeutic formulation to a viscosity less than the viscosity of a control formulation. In embodiments, the therapeutic formulation has an improved stability when compared to the control formulation. In embodiments, the excipient compound is essentially pure.

Further disclosed herein are methods of reducing pain at an injection site of a therapeutic protein in a mammal in need thereof, comprising: administering a liquid therapeutic formulation by injection, wherein the therapeutic formulation comprises a therapeutically effective amount of the therapeutic protein, wherein the formulation further comprises an pharmaceutically acceptable excipient compound selected from the group consisting of local injectable anesthetic compounds, wherein the pharmaceutically acceptable excipient compound is added to the formulation in a viscosity-reducing amount; and wherein the mammal experiences less pain with administration of the therapeutic formulation comprising the excipient compound than that with administration of a control therapeutic formulation, wherein the control therapeutic formulation does not contain the excipient compound and is otherwise identical to the therapeutic formulation.

Disclosed herein, in embodiments, are methods of improving stability of a liquid protein formulation, comprising: preparing a liquid protein formulation comprising a therapeutic protein and an excipient compound selected from the group selected from the group consisting of hindered amines, anionic aromatics, functionalized amino acids, oligopeptides, and short-chain organic acids, and low molecular weight aliphatic polyacids, wherein the liquid protein formulation demonstrates improved stability compared to a control liquid protein formulation, wherein the control liquid protein formulation does not contain the excipient compound and is otherwise identical to the liquid protein formulation. The stability of the liquid formulation can be a cold storage conditions stability, a room temperature stability or an elevated temperature stability.

Also disclosed herein, in embodiments, are liquid formulations comprising a protein and an excipient compound selected from the group consisting of hindered amines, anionic aromatics, functionalized amino acids, oligopeptides, short-chain organic acids, and low molecular weight aliphatic polyacids, wherein the presence of the excipient compound in the formulation results in improved protein-protein interaction characteristics as measured by the protein diffusion interaction parameter kD, or the second virial coefficient B22. In embodiments, the formulation is a therapeutic formulation, and comprises a therapeutic protein. In embodiments, the formulation is a non-therapeutic formulation, and comprises a non-therapeutic protein.

Further disclosed herein, in embodiments, are methods of improving a protein-related process comprising providing the liquid formulation described above, and employing it in a processing method. In embodiments, the processing method includes filtration, pumping, mixing, centrifugation, membrane separation, lyophilization, or chromatography. In embodiments, the processing method is selected from the group consisting of cell culture harvest, chromatography, viral inactivation, and filtration. In embodiments, the processing method is a chromatography process or a filtration process. In embodiments, the filtration process is a virus filtration process or an ultrafiltration/diafiltration process.

Also disclosed herein are methods of improving a parameter of a protein-related process, comprising providing a viscosity-reducing excipient additive comprising at least one excipient compound selected from the group consisting of hindered amines, anionic aromatics, functionalized amino acids, oligopeptides, short-chain organic acids, low molecular weight aliphatic polyacids, and diones and sulfones, and adding a viscosity-reducing amount of the at least one excipient compound to a carrier solution for the protein-related process, wherein the carrier solution contains a protein of interest, thereby improving the parameter. In embodiments, the parameter can be selected from the group consisting of cost of protein production, amount of protein production, rate of protein production, and efficiency of protein production. The parameter can be a proxy parameter. In embodiments, the protein-related process is an upstream processing process. The carrier solution for the upstream processing process can be a cell culture medium. In embodiments, if the carrier solution is a cell culture medium, the step of adding the excipient additive to the carrier solution comprises a first substep of adding the excipient additive to a supplemental medium to form an excipient-containing supplemental medium, and a second substep of adding the excipient-containing supplemental medium to the cell culture medium. In other embodiments, the protein-related process is a downstream processing process. The downstream process can be a chromatography process, and the chromatography process can be a Protein-A chromatography process. In embodiments, the chromatography process recovers the protein of interest, wherein the protein of interest is characterized by an improved protein-related parameter selected from the group consisting of improved purity, improved yield, fewer particles, less misfolding, or less aggregation, as compared to a control solution. In embodiments, the improved protein-related parameter is improved yield of the protein of interest from the chromatography process. In other embodiments, the protein-related process is a process selected from the group consisting of filtration, injection, syringing, pumping, mixing, centrifugation, membrane separation, and lyophilization, and the selected process can require less force than a control process. In embodiments, the protein-related process is selected from the group consisting of a cell culture process, a cell culture harvesting process, a chromatography process, a viral inactivation process, and a filtration process. In embodiments, the protein-related process is the viral inactivation process, and the viral inactivation process is conducted at a pH level of about 2.5 to about 5.0, or the viral inactivation process is conducted at a higher pH than the control process. In other embodiments, the protein-related process is the filtration process. The filtration process can be a virus removal filtration process or an ultrafiltration/diafiltration process. The filtration process can be characterized by an improved filtration-related parameter. The improved filtration-related parameter can be a faster filtration rate than the filtration rate of the control solution. The improved filtration-related parameter can be a production of a smaller amount of aggregated protein than the amount of aggregated protein produced by a control filtration process. The improved filtration-related parameter can be a higher mass transfer efficiency than the mass transfer efficiency of the control filtration process. The improved filtration-related parameter can be a higher concentration or a higher yield of the target protein than a concentration or yield of the target protein produced by the control filtration process.

Further disclosed herein are methods as described above, wherein the viscosity-reducing excipient additive comprises two or more excipient compounds. In embodiments, the at least one excipient compound is a hindered amine. In embodiments, the at least one excipient compound is selected from the group consisting of caffeine, saccharin, acesulfame potassium, aspartame, theophylline, taurine, 1-methyl-2-pyrrolidone, 2-pyrrolidinone, niacinamide, and imidazole. In embodiments, the at least one excipient compound is selected from the group consisting of caffeine, taurine, niacinamide, and imidazole. In embodiments, the at least one excipient compound is an anionic aromatic excipient, and, in some embodiments, the anionic aromatic excipient can be 4-hydroxybenzenesulfonic acid. In embodiments, the viscosity-reducing amount is between about 1 mg/mL to about 100 mg/mL of the at least one excipient compound, or the viscosity-reducing amount is between about 1 mM to about 400 mM of the at least one excipient compound, or the viscosity-reducing amount is an amount from about 2 mM to about 150 mM. In embodiments, the carrier solution comprises an additional agent selected from the group consisting of preservatives, sugars, polysaccharides, arginine, proline, surfactants, stabilizers, and buffers. In embodiments, the protein of interest is a therapeutic protein, and the therapeutic protein can be a recombinant protein, or can be selected from the group consisting of a monoclonal antibody, a polyclonal antibody, an antibody fragment, a fusion protein, a PEGylated protein, an antibody-drug conjugate, a synthetic polypeptide, a protein fragment, a lipoprotein, an enzyme, and a structural peptide. In embodiments, the methods further comprise a step of adding a second viscosity-reducing excipient to the carrier solution, wherein the step of adding the second viscosity-reducing compound adds an additional improvement to the parameter.

In addition, carrier solutions are disclosed herein, comprising a liquid medium in which is dissolved a protein of interest, and a viscosity-reducing additive, wherein the carrier solution has a lower viscosity that that of a control solution. The carrier solution can further comprise an additional agent selected from the group consisting of preservatives, sugars, polysaccharides, arginine, proline, surfactants, stabilizers, and buffers.

Furthermore, the disclosure relates to stability-enhanced formulations, comprising a therapeutic protein and a stability-improving amount of a stabilizing excipient, wherein the stabilized-enhanced formulation is characterized by an improved stability parameter in comparison to a control formulation otherwise identical to the stability-enhanced formulation but lacking the stabilizing excipient. In embodiments, the therapeutic protein is an antibody, and the antibody can be an antibody-drug conjugate. The stabilizing excipient can be a hindered amine compound, an anionic aromatic compound, a functionalized amino acid compound, an oligopeptide, a short-chain organic acid, a low molecular weight polyacid, a dione compound or a sulfone compound, zwitterionic compound, or a crowding agent with hydrogen bonding elements. In embodiments, the stabilizing excipient can be added to the formulation in an amount of about 1 mM to about 500 mM, or in an amount of about 5 mM to about 250 mM, or in an amount of about 10 mM to about 100 mM, or in an amount of about 5 mg/mL to about 50 mg/mL. The improved stability parameter can be thermal storage stability, for example, wherein the thermal storage stability is improved at a temperature between about 10° C. and 30° C. In embodiments, the improved stability parameter is improved freeze/thaw stability or improved shear stability. In embodiments, the stability-enhanced formulation has a reduced number of particles in comparison to the control. In embodiments, the stability-enhanced formulation has an improved biological activity in comparison to the control.

Also disclosed herein are methods of improving stability of a therapeutic formulation, comprising adding a stability-improving amount of a stabilizing excipient to the therapeutic formulation and thereby improving the stability of the therapeutic formulation, wherein the stability of the therapeutic formulation is measured in comparison to the stability of a control formulation otherwise identical to the therapeutic formulation but lacking the stabilizing excipient. The stabilizing excipient can be a hindered amine, an anionic aromatic compound, a functionalized amino acid, an oligopeptide, a short chain organic acid, a low molecular weight polyacid, a dione, a sulfone, a zwitterionic compound or a crowding agent with hydrogen bonding elements. In embodiments, the step of measuring the stability of the therapeutic formulation can comprise measuring a stability-related parameter, for example a parameter selected from the group consisting of thermal storage stability, freeze/thaw stability, and shear stability. In embodiments, the therapeutic formulation comprises a therapeutic protein, which can be an antibody, and the antibody can be an antibody-drug conjugate. Further disclosed herein are methods of improving a parameter of a protein-related process, comprising adding a stability-improving amount of a stabilizing excipient to a carrier solution for the protein-related process, wherein the carrier solution contains a protein of interest, thereby improving the parameter, where the protein of interest can be a therapeutic protein. In embodiments, the parameter can be selected from the group consisting of cost of protein production, amount of protein production, rate of protein production, and efficiency of protein production.

In other embodiments, methods are disclosed herein for improving stability of a therapeutic formulation, comprising preparing a liquid formulation comprising a therapeutic protein, lyophilizing the liquid formulation to produce a lyophilized formulation, reconstituting the lyophilized formulation to produce the therapeutic formulation, and adding an effective amount of a lyo-enhancing excipient to the therapeutic formulation, wherein the therapeutic formulation has improved stability in comparison to the stability of a control formulation otherwise identical to the therapeutic formulation but lacking the lyo-enhancing excipient. In embodiments, the therapeutic protein is a therapeutic antibody, which can be infliximab or ipilimumab. In embodiments, the lyo-enhancing excipient is caffeine. In embodiments, the method further comprises adding nicotinamide to the therapeutic formulation. Further disclosed herein are methods for improving stability of a therapeutic formulation, comprising preparing a liquid formulation comprising a therapeutic protein, adding an effective amount of a lyo-enhancing excipient to the liquid formulation, lyophilizing the liquid formulation to produce a lyophilized formulation, and reconstituting the lyophilized formulation to produce the therapeutic formulation, wherein the therapeutic formulation has improved stability in comparison to the stability of a control formulation otherwise identical to the therapeutic formulation but lacking the lyo-enhancing excipient. Also disclosed herein are methods of reducing the viscosity of a therapeutic formulation, comprising preparing a liquid formulation comprising a therapeutic protein, lyophilizing the liquid formulation to produce a lyophilized formulation, adding a viscosity-reducing amount of a lyo-enhancing excipient to the liquid formulation before, during, or after the step of lyophilizing the liquid formulation, and reconstituting the lyophilized formulation to produce the therapeutic formulation, wherein the viscosity of the therapeutic formulation is reduced in comparison to the stability of a control formulation otherwise identical to the therapeutic formulation but lacking the lyo-enhancing excipient. In embodiments, the step of adding the viscosity-reducing amount of the lyo-enhancing excipient to the liquid formulation is performed before the step of lyophilizing the liquid formulation. Additionally, methods are disclosed herein for improving a parameter of a lyophilization process, comprising preparing a liquid formulation comprising a therapeutic protein, adding a lyo-improving excipient to the liquid formulation, and treating the liquid formulation with the lyophilization process, wherein the parameter of the lyophilization process is improved when compared to a comparable parameter of a comparable lyophilization process performed in the absence of the lyo-improving excipient. In embodiments, the parameter can be selected from the group consisting of drying time, reconstitution speed, process efficiency, energy savings, ability to lyophilize at higher concentration than would be possible without the excipients, improved cake quality, reduced particle formation, and ability to lyophilize at higher concentration than is possible in the absence of the lyo-improving excipient.

The invention also includes a method of improving the stability of a reconstituted formulation of a therapeutic protein, comprising the steps of: preparing a liquid formulation comprising the therapeutic protein; lyophilizing the liquid formulation to produce a lyophilized formulation; and reconstituting the lyophilized formulation with a reconstituting solvent to produce the reconstituted formulation, wherein the method further comprises adding an effective amount of a lyo-enhancing excipient to the liquid formulation before or during the lyophilizing step; and/or adding an effective amount of a lyo-enhancing excipient to the lyophilized formulation after the lyophilizing step; wherein the reconstituted formulation has improved stability in comparison to the stability of a control formulation otherwise identical to the reconstituted formulation but lacking the lyo-enhancing excipient. In certain aspects, the therapeutic protein is a therapeutic antibody, for example, infliximab or ipilimumab. In additional aspects, the lyo-enhancing excipient is a hindered amine, for example, caffeine and/or nicotinamide. In certain aspects, the method comprises adding an effective amount of a lyo-enhancing excipient to the liquid formulation before or during the lyophilizing step, and optionally further comprises adding an effective amount of a second lyo-enhancing excipient to the lyophilized formulation. The lyo-enhancing excipient added before or during the lyophilizing step can be the same as or different from the second lyo-enhancing excipient. In certain aspects, wherein a second lyo-enhancing excipient is added, the lyo-enhancing excipient can be added before the step of lyophilizing and the second lyo-enhancing excipient is added to a reconstituting solvent. In some embodiments, the lyo-enhancing excipient is caffeine and the second lyo-enhancing excipient is nicotinamide. In certain aspects, the method comprises adding an effective amount of a lyo-enhancing excipient to the lyophilized formulation; for example, the lyo-enhancing excipient can be added to the reconstituting solvent prior to the step of reconstituting the liquid formulation. The lyo-enhancing excipient can, for example, be added to the reconstituting solvent in an amount such that the concentration of the lyo-enhancing excipient in the reconstituted formulation is between about 0.1 mg/mL and about 200 mg/mL, between about 1 mg/mL and about 100 mg/mL, or between about 2 mg/mL and about 50 mg/mL. In additional aspects, the lyo-enhancing excipient can be added to the reconstituting solvent in an amount such that the mass concentration of the lyo-enhancing excipient in the reconstituted formulation is between about 1% and about 10%, between about 2% and about 9%, or between about 3% and about 8%, of the mass concentration of the therapeutic protein in the reconstituted formulation. Thus, for example, if the mass concentration of the therapeutic protein in the reconstituted formulation is 100 mg/mL, then the mass concentration of the lyo-enhancing excipient that is between about 1% and 10% of that of the therapeutic protein is between about 1 to 10 mg/mL. The methods described herein can further comprise adding an additional excipient selected from the group consisting of sugars, polyols, polymers, buffers, inorganic salts, amino acids, chelating agents, and surfactants to the formulation before, during, or after the step of lyophilizing; for example, the additional excipient can be selected from the group consisting of trehalose, sucrose, mannitol, glucose, glycine, and polyvinylpyrrolidone to the formulation before, during, or after the step of lyophilizing.

The invention additionally encompasses a method of improving the stability of a freeze concentrate formulation of a therapeutic protein, comprising the steps of: preparing a liquid formulation comprising the therapeutic protein; adding an effective amount of a lyo-enhancing excipient to the liquid formulation before the freezing step; and freezing the liquid formulation to prepare a freeze concentrate formulation, wherein the freeze concentrate formulation has improved stability in comparison to the stability of a control formulation otherwise identical to the freeze concentrate formulation but lacking the lyo-enhancing excipient.

In additional aspects, the invention includes a method of reducing the viscosity of a reconstituted formulation of a therapeutic protein, comprising the steps of: preparing a liquid formulation comprising the therapeutic protein; lyophilizing the liquid formulation to produce a lyophilized formulation; and reconstituting the lyophilized formulation with a reconstituting solvent to produce the reconstituted formulation, wherein the method further comprises adding an effective amount of a lyo-enhancing excipient to the liquid formulation before or during the lyophilizing step, and/or wherein the method further comprises adding an effective amount of a lyo-enhancing excipient to the lyophilized formulation after the lyophilizing step, wherein the viscosity of the reconstituted formulation is reduced in comparison to the stability of a control formulation otherwise identical to the reconstituted formulation but lacking the lyo-enhancing excipient. In certain aspects, the therapeutic protein is a therapeutic antibody, for example, infliximab or ipilimumab. In yet additional aspects, the lyo-enhancing excipient is a hindered amine; for example, caffeine and/or nicotinamide. In certain aspects, the method comprises adding an effective amount of a lyo-enhancing excipient to the liquid formulation before or during the lyophilizing step, and optionally further comprises adding an effective amount of a second lyo-enhancing excipient to the lyophilized formulation. The lyo-enhancing excipient added before or during the lyophilizing step can be the same aa or different from the second lyo-enhancing excipient. In some embodiments where a second lyo-enhancing excipient is added, the lyo-enhancing excipient can be added before the lyophilizing step and the second lyo-enhancing excipient can be added to the reconstituting solvent. In certain examples, the lyo-enhancing excipient is caffeine and the second lyo-enhancing excipient is nicotinamide. In additional embodiments, the method comprises adding an effective amount of a lyo-enhancing excipient to the lyophilized formulation. For example, the lyo-enhancing excipient can be added to the reconstituting solvent prior to the step of reconstituting the liquid formulation. For example, the lyo-enhancing excipient can be added to the reconstituting solvent in an amount such that the concentration of the lyo-enhancing excipient in the reconstituted solvent is between about 0.1 mg/mL and about 200 mg/mL, between about 1 mg/mL and about 100 mg/mL, or between about 2 mg/mL and about 50 mg/mL. In yet additional aspects, the lyo-enhancing excipient is added to the reconstituting solvent in an amount such that the mass concentration of the lyo-enhancing excipient in the reconstituted formulation is between about 1% and about 10%, between about 2% and about 9%, or between about 3% and about 8% of the mass concentration of the therapeutic protein in the reconstituted formulation. The method can also comprise adding an additional excipient selected from the group consisting of sugars, polyols, polymers, buffers, inorganic salts, amino acids, chelating agents, and surfactants to the formulation before, during, or after the step of lyophilizing; for example, the additional excipient can be selected from the group consisting of trehalose, sucrose, mannitol, glucose, glycine, and polyvinylpyrrolidone to the formulation before, during, or after the step of lyophilizing.

The invention also encompasses a method of improving a parameter of a lyophilization process, comprising preparing a liquid formulation comprising a therapeutic protein, adding a lyo-improving excipient to the liquid formulation, and treating the liquid formulation with the lyophilization process, wherein the parameter of the lyophilization process is improved when compared to a comparable parameter of a comparable lyophilization process performed in the absence of the lyo-improving excipient. In some aspects, the parameter is selected from the group consisting of drying time, reconstitution speed, process efficiency, energy savings, improved cake quality, reduced particle formation, and ability to lyophilize or reconstitute at higher concentration than is possible in the absence of the lyo-improving excipient.

Also described herein is a reduced viscosity formulation, wherein the formulation is a reconstituted formulation of a lyophilizate that comprises a therapeutic protein, the formulation further comprising a liquid medium and a lyo-enhancing excipient, wherein the therapeutic protein is present in the formulation in an amount of at least about 100 mg/ml; wherein the lyo-enhancing excipient is caffeine or nicotinamide, wherein the lyo-enhancing excipient is present in the composition in an amount between about 2 and about 100 mg/ml or between about 2 and about 50 mg/ml; wherein the viscosity of the reduced viscosity formulation is at least about 30% lower than the viscosity of a control formulation otherwise identical to the reduced viscosity formulation but lacking the lyo-enhancing excipient. The therapeutic protein can be a therapeutic antibody; for example, infliximab or ipilimumab. In certain aspects, the lyo-enhancing excipient is caffeine. In some aspects, the formulation, further comprises a second lyo-enhancing excipient; for example, nicotinamide.

The invention also includes a therapeutic formulation in lyophilized form, comprising a therapeutic antibody and further comprising caffeine and an additional excipient in the following amounts for every 100 mg of infliximab (wherein the additional excipient is a sugar or a polyol): the sugar or polyol at the amount of about 50 mg to about 100 mg; and the caffeine at the amount of about 2 mg to about 10 mg. In certain aspects, the therapeutic antibody is infliximab or ipilimumab. The sugar or polyol can be selected from the group consisting of sucrose, trehalose, glucose, and sorbitol. In certain aspects, the amount of caffeine for every 100 mg of infliximab is between about 4 mg and about 8 mg, or between about 1 and about 10 mg. The formulation can further comprise a buffer, such as a phosphate or a histidine buffer. In additional aspects, the formulation further comprises a surfactant, for example, a polysorbate and the amount of polysorbate is between about 0.05 mg and about 1 mg; for example, the amount of polysorbate can be between about 0.1 mg and 0.5 mg. In yet further aspects, the formulation further comprises an additional agent selected from the group consisting of bulking and crystalline agents, and amino acids; for example, mannitol and glycine.

The invention also includes a reconstituted therapeutic formulation of a lyophilizate comprising infliximab, wherein the formulation comprises: infliximab in an amount between about 100 mg/mL and about 200 mg/mL; a sugar or polyol in the amount of between about 50 mg/mL to about 200 mg/mL; and caffeine in the amount of between about 25 mg to about 100 mg. In additional aspects, the amount of infliximab is between about 125 mg/mL and about 175 mg/mL, and/or the amount of sugar or polyol is between about 90 mg/mL and 120 mg/mL. In certain aspects, the sugar or polyol is selected from the group consisting of sucrose, trehalose, glucose, and sorbitol. The amount of caffeine can be between about 30 mg and about 80 mg. The amount of nicotinamide can be between about 10 mM and about 100 mM, or between about 25 mM and 50 mM. In certain embodiments, the reconstituted therapeutic formulation further comprises a buffer, wherein the pH of the reconstituted lyophilized therapeutic formulation is between 5.0 and 7.5 or between 5.5 and 6.5. The reconstituted therapeutic formulation can further comprise a surfactant; for example, in the amount between about 50 ppm and about 500 ppm or between about 100 ppm and 200 ppm.

### BRIEF DESCRIPTION OF THE FIGURES

FIG. 1 shows a graph of particle size distributions for solutions of a monoclonal antibody under stressed and non-stressed conditions, as evaluated by Dynamic Light Scattering. The data curves in FIG. 1 have a baseline offset to allow comparison: the curve for Sample 1-A is offset by 100 intensity units and the curve for Sample 1-FT is offset by 200 intensity units in the Y-axis.

FIG. 2 shows a graph measuring sample diameter vs. multimodal size distribution for several molecular populations, as evaluated by Dynamic Light Scattering. The data curves in FIG. 2 have a baseline offset to allow comparison: the curve for Sample 2-A is offset by 100 intensity units and the curve for Sample 2-FT is offset by 200 intensity units in the Y-axis.

FIG. 3 shows a size exclusion chromatogram of monoclonal antibody solutions with a main monomer peak at 8-10 minutes retention time. The data curves in FIG. 3 have a baseline offset to allow comparison: the curves for Samples 2-C, 2-A, and 2-FT are offset in the Y-axis direction.

FIG. 4 presents a block diagram showing the steps in a fermentation process (an “upstream processing”) for producing therapeutic proteins, for example monoclonal antibodies.

FIG. 5 presents a block diagram showing the steps in a purification process (a “downstream processing”) for producing therapeutic proteins, for example monoclonal antibodies.

FIGS. 6A, 6B, 6C, 6D are graphs that represent CEX-HPLC chromatograms showing charge variants in infliximab (FIGS. 6A and 6B) and ipilimumab (FIGS. 6C and 6D) formulations reconstituted after lyophilization and after being stored at 40° C. for 4 weeks.

FIGS. 7A, 7B, 7C, 7D are graphs that show amounts of aggregates measured via SEC in formulations of infliximab (FIGS. 7A and 7B) and ipilimumab (FIGS. 7C and 7D), with measurements made to formulations reconstituted after lyophilization, and after being stored at 40° C. for 4 weeks.

### DETAILED DESCRIPTION OF THE INVENTION

Disclosed herein are formulations and methods for their production that permit the delivery of protein solutions, including concentrated protein solutions. In embodiments, the approaches disclosed herein can yield a lower viscosity liquid formulation or a higher concentration of therapeutic or nontherapeutic proteins in the liquid formulation, as compared to traditional protein solutions.

In embodiments, the approaches disclosed herein can yield a liquid formulation having improved stability when compared to a traditional protein solution. In one aspect, a stable formulation is one in which the protein contained therein substantially retains its physical and chemical stability or integrity and its therapeutic or nontherapeutic efficacy upon exposure to a stress condition. In another aspect, a stable formulation is one in which the protein contained therein substantially retains its soluble, monomeric, or non-aggregated state. As used herein, a stress condition is a physical or chemical condition that adversely affects a protein in a formulation. Advantageously, a stable formulation can also offer protection against aggregation or precipitation of the proteins dissolved therein.

Examples of physical stress conditions include physical perturbations such as mechanical shear, contact with air/water interfaces, freeze-thaw cycles, prolonged storage under storage conditions (whether cold storage conditions, room temperature conditions, or elevated temperature storage conditions) or exposure to other denaturing conditions. For example, the cold storage conditions can entail storage in a refrigerator or freezer. In some examples, cold storage conditions can entail storage at a temperature of 10° C. or less. In additional examples, the cold storage conditions entail storage at a temperature from about 2° to about 10° C. In other examples, the cold storage conditions entail storage at a temperature of about 4° C. In additional examples, the cold storage conditions entail storage at freezing temperature such as about −20° C. or lower. In another example, cold storage conditions entail storage at a temperature of about −80° C. to about 0° C. The room temperature storage conditions can entail storage at ambient temperatures, for example, from about 10° C. to about 30° C. Elevated storage conditions can entail storage at a temperature greater than about 30° C. Elevated temperature stability, for example at temperatures from about 30° C. to about 50° C., can be used as part an accelerated aging study to predict the long-term storage at typical ambient (10-30° C.) conditions. Stress conditions can also include chemical perturbations, such as changes in pH, that can affect the stability or integrity of a protein in the formulation, for example by affecting its tertiary structure.

It is well known to those skilled in the art of polymer science and engineering that proteins in solution tend to form entanglements, which can limit the translational mobility of the entangled chains and interfere with the protein's therapeutic or nontherapeutic efficacy. In embodiments, excipient compounds as disclosed herein can suppress protein clustering due to specific interactions between the excipient compound and the target protein in solution. Excipient compounds as disclosed herein can be natural or synthetic, and desirably are substances that the FDA generally recognizes as safe (“GRAS”).

### 1. Definitions

For the purpose of this disclosure, the term “protein” refers to a sequence of amino acids having a chain length long enough to produce a discrete tertiary structure, typically having a molecular weight between 1-3000 kDa. In some embodiments, the molecular weight of the protein is between about 50-200 kDa; in other embodiments, the molecular weight of the protein is between about 20-1000 kDa or between about 20-2000 kDa. In contrast to the term “protein,” the term “peptide” refers to a sequence of amino acids that does not have a discrete tertiary structure. A wide variety of biopolymers are included within the scope of the term “protein.” For example, the term “protein” can refer to therapeutic or non-therapeutic proteins, including antibodies, aptamers, fusion proteins, PEGylated proteins, synthetic polypeptides, protein fragments, lipoproteins, enzymes, structural peptides, and the like.

a. Therapeutic Biopolymers and Related Definitions

Those biopolymers, including proteins, having therapeutic effects may be termed “therapeutic biopolymers.” Those proteins having therapeutic effects may be termed “therapeutic proteins.”

As non-limiting examples, therapeutic proteins can include mammalian proteins such as hormones and prohormones (e.g., insulin and proinsulin, glucagon, calcitonin, thyroid hormones (T3 or T4 or thyroid-stimulating hormone), parathyroid hormone, follicle-stimulating hormone, luteinizing hormone, growth hormone, growth hormone releasing factor, and the like); clotting and anti-clotting factors (e.g., tissue factor, von Willebrand's factor, Factor VIIIC, Factor IX, protein C, plasminogen activators (urokinase, tissue-type plasminogen activators), thrombin); cytokines, chemokines, and inflammatory mediators; interferons; colony-stimulating factors; interleukins (e.g., IL-1 through IL-10); growth factors (e.g., vascular endothelial growth factors, fibroblast growth factor, platelet-derived growth factor, transforming growth factor, neurotrophic growth factors, insulin-like growth factor, and the like); albumins; collagens and elastins; hematopoietic factors (e.g., erythropoietin, thrombopoietin, and the like); osteoinductive factors (e.g., bone morphogenetic protein); receptors (e.g., integrins, cadherins, and the like); surface membrane proteins; transport proteins; regulatory proteins; antigenic proteins (e.g., a viral component that acts as an antigen); and antibodies.

In certain embodiments, the therapeutic protein can be an antibody. The term “antibody” is used herein in its broadest sense, to include as non-limiting examples monoclonal antibodies (including, for example, full-length antibodies with an immunoglobulin Fc region), single-chain molecules, bi-specific and multi-specific antibodies, diabodies, antibody-drug conjugates, antibody compositions having polyepitopic specificity, polyclonal antibodies (such as polyclonal immunoglobulins used as therapies for immune-compromised patients), and fragments of antibodies (including, for example, Fc, Fab, Fv, nanobodies, and F(ab′)2). Antibodies can also be termed “immunoglobulins.” An antibody is understood to be directed against a specific protein or non-protein “antigen,” which is a biologically important material; the administration of a therapeutically effective amount of an antibody to a patient can complex with the antigen, thereby altering its biological properties so that the patient experiences a therapeutic effect.

In embodiments, the antibodies can be antibody-drug conjugates (ADCs). Antibody-drug conjugates are a category of therapeutic proteins that combine the highly particularized targeting capabilities of antibodies with a therapeutically active compound such as a cytotoxic compound: ADCs are composed of the antibody that is linked via a biodegradable chemical linker to the therapeutically active agent. In more detail, the ADC can include a human or humanized mAb that is specific for an antigen that is expressed on an abnormal “target” cell, but that has minimal or no expression on normal cells. The ADC further includes a potent pharmaceutical agent, such as a cytotoxic agent that can destroy the target cells; such agents are typically toxic systemically, so that they are not suitable for generalized, systemic administration. The targeting capabilities of the mAb component of the ADC allow the pharmaceutical agent to be directed specifically to the target cells, become absorbed by the target cells, and exert its effects within those cells, all without being distributed systemically. To form an ADC, the mAb is linked to the pharmaceutical agent with labile bonds that are stable in the extracellular milieu (e.g., in intravenous and interstitial circulation), but that are degraded when the ADC is internalized into the cell. As the linkage between the ADC and the pharmaceutical agent is degraded, the agent is released inside the cell to exert its effects on the cell ADCs are especially suitable for use with cytotoxic agents, especially where these compounds are too toxic for use as stand-alone treatments. In cancer chemotherapy, for example, some of the agents selected for use in ADCs are several orders of magnitude more toxic than traditional anticancer agents. Examples include anti-microtubule agents, alkylating agents and DNA minor groove binding agents, which may be too toxic to administer successfully but which can be targeted at cancer cells using the specificity of a mAb that binds with an antigen expressed only by the cancer cell. Once the ADC localizes to the tumor and binds to the target cell antigen on the surface, the complex can be internalized into the cell in a vesicle. The internalized vesicles fuse with each other and enter the endosome-lysosome pathway, where they encounter proteases that digest the mAb and/or the linker molecule, thereby releasing the pharmaceutical payload. The payload (e.g., the cytotoxic agent) then crosses the lysosomal membrane to enter the cytoplasm and/or the nucleus, where it exerts its pharmaceutical (e.g., cytotoxic) effects. This focused delivery of highly potent pharmaceutical compounds maximizes their intended therapeutic effect while minimizing the exposure of normal tissues to these agents. Formulations comprising ADCs are suitable for intravenous or local administration so that the ADC reaches the target cells to be treated.

In certain embodiments, the therapeutic proteins are PEGylated, meaning that they comprise poly(ethylene glycol) (“PEG”) and/or poly(propylene glycol) (“PPG”) units. PEGylated proteins, or PEG-protein conjugates, have found utility in therapeutic applications due to their beneficial properties such as solubility, pharmacokinetics, pharmacodynamics, immunogenicity, renal clearance, and stability. Non-limiting examples of PEGylated proteins are PEGylated interferons (PEG-IFN), PEGylated anti-VEGF, PEG protein conjugate drugs, Adagen, Pegaspargase, Pegfilgrastim, Pegloticase, Pegvisomant, PEGylated epoetin-β, and Certolizumab pegol.

PEGylated proteins can be synthesized by a variety of methods such as a reaction of protein with a PEG reagent having one or more reactive functional groups. The reactive functional groups on the PEG reagent can form a linkage with the protein at targeted protein sites such as lysine, histidine, cysteine, and the N-terminus. Typical PEGylation reagents have reactive functional groups such as aldehyde, maleimide, or succinimide groups that have specific reactivity with targeted amino acid residues on proteins. The PEGylation reagents can have a PEG chain length from about 1 to about 1000 PEG and/or PPG repeating units. Other methods of PEGylation include glyco-PEGylation, where the protein is first glycosylated and then the glycosylated residues are PEGylated in a second step. Certain PEGylation processes are assisted by enzymes like sialyltransferase and transglutaminase.

While the PEGylated proteins can offer therapeutic advantages over native, non-PEGylated proteins, these materials can have physical or chemical properties that make them difficult to purify, dissolve, filter, concentrate, and administer. The PEGylation of a protein can lead to a higher solution viscosity compared to the native protein, and this generally requires the formulation of PEGylated protein solutions at lower concentrations.

It is desirable to formulate protein therapeutics in stable, low viscosity solutions so they can be administered to patients in a minimal injection volume. For example, the subcutaneous (SC) or intramuscular (IM) injection of drugs generally requires a small injection volume, preferably less than 2 mL. The SC and IM injection routes are well suited to self-administered care, and this is a less costly and more accessible form of treatment compared with intravenous (IV) injection which is only conducted under direct medical supervision. Formulations for SC or IM injection require a low solution viscosity, generally below 30 cP, and preferably below 20 cP, to allow easy flow of the therapeutic solution through a narrow-gauge needle. This combination of small injection volume and low viscosity requirements present a challenge to the use of PEGylated protein therapeutics in SC or IM injection routes.

Formulations containing therapeutic proteins in therapeutically effective amounts may be termed “therapeutic formulations.” The therapeutic protein contained in a therapeutic formulation may also be termed its “protein active ingredient.” Typically, a therapeutic formulation comprises a therapeutically effective amount of a protein active ingredient and an excipient, with or without other optional components. As used herein, the term “therapeutic” includes both treatments of existing disorders and preventions of disorders. Therapeutic proteins include, for example, proteins such as clotting factors, antibodies, enzymes, fusion proteins, cytokines, peptides, antibody-drug conjugates, neurotoxins, PEGylated biologics, and the like; in embodiments, therapeutic proteins include but are not limited to proteins such as bevacizumab, trastuzumab, adalimumab, infliximab, etanercept, darbepoetin alfa, epoetin alfa, cetuximab, filgrastim, and rituximab. Other therapeutic proteins will be familiar to those having ordinary skill in the art.

A “treatment” includes any measure intended to cure, heal, alleviate, improve, remedy, or otherwise beneficially affect the disorder, including preventing or delaying the onset of symptoms and/or alleviating or ameliorating symptoms of the disorder. Those patients in need of a treatment include both those who already have a specific disorder, and those for whom the prevention of a disorder is desirable. A disorder is any condition that alters the homeostatic wellbeing of a mammal, including acute or chronic diseases, or pathological conditions that predispose the mammal to an acute or chronic disease. Non-limiting examples of disorders include cancers, metabolic disorders (e.g., diabetes), allergic disorders (e.g., asthma), dermatological disorders, cardiovascular disorders, respiratory disorders, hematological disorders, musculoskeletal disorders, inflammatory or rheumatological disorders, autoimmune disorders, gastrointestinal disorders, urological disorders, sexual and reproductive disorders, neurological disorders, and the like. The term “mammal” for the purposes of treatment can refer to any animal classified as a mammal, including humans, domestic animals, pet animals, farm animals, sporting animals, working animals, and the like. A “treatment” can therefore include both veterinary and human treatments. For convenience, the mammal undergoing such “treatment” can be referred to as a “patient.” In certain embodiments, the patient can be of any age, including fetal animals in utero.

In embodiments, a treatment involves providing a therapeutically effective amount of a therapeutic formulation to a mammal in need thereof. A “therapeutically effective amount” is at least the minimum concentration of the therapeutic protein administered to the mammal in need thereof, to effect a treatment of an existing disorder or a prevention of an anticipated disorder (either such treatment or such prevention being a “therapeutic intervention”). Therapeutically effective amounts of various therapeutic proteins that may be included as active ingredients in the therapeutic formulation may be familiar in the art; or, for therapeutic proteins discovered or applied to therapeutic interventions hereafter, the therapeutically effective amount can be determined by standard techniques carried out by those having ordinary skill in the art, using no more than routine experimentation.

b. Non-Therapeutic Biopolymers and Related Definitions

Those proteins used for non-therapeutic purposes (i.e., purposes not involving treatments), such as household, nutrition, commercial, and industrial applications, may be termed “non-therapeutic proteins.” Formulations containing non-therapeutic proteins may be termed “non-therapeutic formulations.” The non-therapeutic proteins can be derived from plant sources, animal sources, or produced from cell cultures; they also can be enzymes or structural proteins. The non-therapeutic proteins can be used in in household, nutrition, commercial, and industrial applications such as catalysts, human and animal nutrition, processing aids, cleaners, and waste treatment.

An important category of non-therapeutic biopolymers is the category of enzymes. Enzymes have a number of non-therapeutic applications, for example, as catalysts, human and animal nutritional ingredients, processing aids, cleaners, and waste treatment agents. Enzyme catalysts are used to accelerate a variety of chemical reactions. Examples of enzyme catalysts for non-therapeutic uses include catalases, oxidoreductases, transferases, hydrolases, lyases, isomerases, and ligases. Human and animal nutritional uses of enzymes include nutraceuticals, nutritive sources of protein, chelation or controlled delivery of micronutrients, digestion aids, and supplements; these can be derived from amylase, protease, trypsin, lactase, and the like. Enzymatic processing aids are used to improve the production of food and beverage products in operations like baking, brewing, fermenting, juice processing, and winemaking. Examples of these food and beverage processing aids include amylases, cellulases, pectinases, glucanases, lipases, and lactases. Enzymes can also be used in the production of biofuels. Ethanol for biofuels, for example, can be aided by the enzymatic degradation of biomass feedstocks such as cellulosic and lignocellulosic materials. The treatment of such feedstock materials with cellulases and ligninases transforms the biomass into a substrate that can be fermented into fuels. In other commercial applications, enzymes are used as detergents, cleaners, and stain lifting aids for laundry, dish washing, surface cleaning, and equipment cleaning applications. Typical enzymes for this purpose include proteases, cellulases, amylases, and lipases. In addition, non-therapeutic enzymes are used in a variety of commercial and industrial processes such as textile softening with cellulases, leather processing, waste treatment, contaminated sediment treatment, water treatment, pulp bleaching, and pulp softening and debonding. Typical enzymes for these purposes are amylases, xylanases, cellulases, and ligninases.

Other examples of non-therapeutic biopolymers include fibrous or structural proteins such as keratins, collagen, gelatin, elastin, fibroin, actin, tubulin, or the hydrolyzed, degraded, or derivatized forms thereof. These materials are used in the preparation and formulation of food ingredients such as gelatin, ice cream, yogurt, and confections; they area also added to foods as thickeners, rheology modifiers, mouthfeel improvers, and as a source of nutritional protein. In the cosmetics and personal care industry, collagen, elastin, keratin, and hydrolyzed keratin are widely used as ingredients in skin care and hair care formulations. Still other examples of non-therapeutic biopolymers are whey proteins such as beta-lactoglobulin, alpha-lactalbumin, and serum albumin. These whey proteins are produced in mass scale as a byproduct from dairy operations and have been used for a variety of non-therapeutic applications.

### 2. Measurements

In embodiments, the protein-containing formulations described herein are resistant to monomer loss as measured by size exclusion chromatography (SEC) analysis. In SEC analysis as used herein, the main analyte peak is generally associated with the target protein contained in the formulation, and this main peak of the protein is referred to as the monomer peak. The monomer peak represents the amount of target protein, e.g., a protein active ingredient, in the monomeric state, as opposed to aggregated (dimeric, trimeric, oligomeric, etc.) or fragmented states. The monomer peak area can be compared with the total area of the monomer, aggregate, and fragment peaks associated with the target protein. Thus, the stability of a protein-containing formulation can be observed by the relative amount of monomer after an elapsed time; an improvement in stability of a protein-containing formulation of the invention can therefore be measured as a higher percent monomer after a certain elapsed time, as compared to the percent monomer in a control formulation that does not contain the excipient.

In embodiments, an ideal stability result is to have from 98 to 100% monomer peak as determined by SEC analysis. In embodiments, an improvement in stability of a protein-containing formulation of the invention can be measured as a higher percent monomer after exposure to a stress condition, as compared to the percent monomer in a control formulation that does not contain the excipient when such control formulation is exposed to the same stress condition. In embodiments, the stress conditions can be a low temperature storage, high temperature storage, exposure to air, exposure to light, exposure to gas bubbles, exposure to shear conditions, or exposure to freeze/thaw cycles.

In embodiments, the protein-containing formulations as described herein are resistant to an increase in protein particle size as measured by dynamic light scattering (DLS) analysis. In DLS analysis as used herein, the particle size of the protein in the protein-containing formulation can be observed as a median particle diameter. Ideally, the median particle diameter of the target protein should be relatively unchanged when subjected to DLS analysis, since the particle diameter represents the active component in the monomeric state, as opposed to aggregated (dimeric, trimeric, oligomeric, etc.) or fragmented states. An increase of the median particle diameter could represent an aggregated protein. Thus, the stability of a protein-containing formulation can be observed by the relative change in median particle diameter after an elapsed time.

In embodiments, the protein-containing formulations as described herein are resistant to forming a polydisperse particle size distribution as measured by DLS analysis. In embodiments, a protein-containing formulation can contain a monodisperse particle size distribution of colloidal protein particles. In embodiments, an ideal stability result is to have less than a 10% change in the median particle diameter compared to the initial median particle diameter of the formulation. In embodiments, an improvement in stability of a protein-containing formulation of the invention can be measured as a lower percent change of the median particle diameter after a certain elapsed time, as compared to the median particle diameter in a control formulation that does not contain the excipient. In embodiments, an improvement in stability of a protein-containing formulation of the invention can be measured as a lower percent change of the median particle diameter after exposure to a stress condition, as compared to the percent change of the median particle diameter in a control formulation that does not contain the excipient when such control formulation is exposed to the same stress condition. In embodiments, the stress conditions can be a low temperature storage, high temperature storage, exposure to air, exposure to light, exposure to gas bubbles, exposure to shear conditions, or exposure to freeze/thaw cycles. In embodiments, an improvement in stability of a protein-containing formulation therapeutic formulation of the invention can be measured as a less polydisperse particle size distribution as measured by DLS, as compared to the polydispersity of the particle size distribution in a control formulation that does not contain the excipient when such control formulation is exposed to the same stress condition.

In embodiments, the protein-containing formulations disclosed herein are resistant to particle formation, denaturation, or precipitation as measured by turbidity, light scattering, and/or particle counting analysis. In turbidity, light scattering, or particle counting analysis, a lower value generally represents a lower number of suspended particles in a formulation. An increase of turbidity, light scattering, or particle counting can indicate that the solution of the target protein is not stable. Thus, the stability of a protein-containing formulation can be observed by the relative amount of turbidity, light scattering, or particle counting after an elapsed time. In embodiments, an ideal stability result is to have a low and relatively constant turbidity, light scattering, or particle counting value. In embodiments, an improvement in stability of a protein-containing formulation as described herein can be measured as a lower turbidity, lower light scattering, or lower particle count after a certain elapsed time, as compared to the turbidity, light scattering, or particle count values in a control formulation that does not contain the excipient. In embodiments, an improvement in stability of a protein-containing formulation as described herein can be measured as a lower turbidity, lower light scattering, or lower particle count after exposure to a stress condition, as compared to the turbidity, light scattering, or particle count in a control formulation that does not contain the excipient when such control formulation is exposed to the same stress condition. In embodiments, the stress conditions can be a low temperature storage, high temperature storage, exposure to air, exposure to light, exposure to gas bubbles, exposure to shear conditions, or exposure to freeze/thaw cycles. In embodiments, the protein-containing formulations as disclosed herein retain a higher percentage of biological activity compared with a control formulation. The biological activity can be observed via a binding assay or via a therapeutic effect in a mammal.

### 3. Therapeutic Formulations

In one aspect, the formulations and methods disclosed herein provide stable liquid formulations of improved or reduced viscosity, comprising a therapeutic protein in a therapeutically effective amount and an excipient compound. In embodiments, the formulation can improve the stability while providing an acceptable concentration of active ingredients and an acceptable viscosity. In embodiments, the formulation provides an improvement in stability when compared to a control formulation; for the purposes of this disclosure, a control formulation is a formulation containing the protein active ingredient that is identical on a dry weight basis in every way to the therapeutic formulation except that it lacks the excipient compound. In embodiments, the formulation provides an improvement in stability under the stress conditions of long term storage, elevated temperatures such as 25-45° C., freeze/thaw conditions, shear or mixing, syringing, dilution, gas bubble exposure, oxygen exposure, light exposure, and lyophilization. In embodiments, improved stability of the protein-containing formulation is in the form of lower percentage of soluble aggregates, particulates, subvisible particles, or gel formation, compared to a control formulation.

It is understood that the viscosity of a liquid protein formulation can be affected by a variety of factors, including but not limited to: the nature of the protein itself (e.g., enzyme, antibody, receptor, fusion protein, etc.); its size, three-dimensional structure, chemical composition, and molecular weight; its concentration in the formulation; the components of the formulation besides the protein; the desired pH range; the storage conditions for the formulation; and the method of administering the formulation to the patient. Therapeutic proteins most suitable for use with the excipient compounds described herein are preferably essentially pure, i.e., free from contaminating proteins. In embodiments, an “essentially pure” therapeutic protein is a protein composition comprising at least 90% by weight of the therapeutic protein, or preferably at least 95% by weight, or more preferably, at least 99% by weight, all based on the total weight of therapeutic proteins and contaminating proteins in the composition. For the purposes of clarity, a protein added as an excipient is not intended to be included in this definition. The therapeutic formulations described herein are intended for use as pharmaceutical-grade formulations, i.e., formulations intended for use in treating a mammal, in such a form that the desired therapeutic efficacy of the protein active ingredient can be achieved, and without containing components that are toxic to the mammal to whom the formulation is to be administered.

In embodiments, the therapeutic formulation contains at least 25 mg/mL of protein active ingredient. In other embodiments, the therapeutic formulation contains at least 100 mg/mL of protein active ingredient. In other embodiments, the therapeutic formulation contains at least 10 mg/mL of protein active ingredient. In other embodiments, the therapeutic formulation contains at least 50 mg/mL of protein active ingredient. In other embodiments, the therapeutic formulation contains at least 200 mg/mL of protein active ingredient. In yet other embodiments, the therapeutic formulation solution contains at least 300 mg/mL of protein active ingredient. Generally, the excipient compounds disclosed herein are added to the therapeutic formulation in an amount between about 5 to about 300 mg/mL. In embodiments, the excipient compound can be added in an amount of about 10 to about 200 mg/mL. In embodiments, the excipient compound can be added in an amount of about 20 to about 100 mg/mL. In embodiments, the excipient can be added in an amount of about 25 to about 75 mg/mL.

Excipient compounds of various molecular weights are selected for specific advantageous properties when combined with the protein active ingredient in a formulation. Examples of therapeutic formulations comprising excipient compounds are provided below. In embodiments, the excipient compound has a molecular weight of <5000 Da. In embodiments, the excipient compound has a molecular weight of <1000 Da. In embodiments, the excipient compound has a molecular weight of <500 Da.

In embodiments, the excipient compounds disclosed herein are added to the therapeutic formulation in a viscosity-reducing amount. In embodiments, a viscosity-reducing amount is the amount of an excipient compound that reduces the viscosity of the formulation at least 10% when compared to a control formulation; for the purposes of this disclosure, a control formulation is a formulation containing the protein active ingredient that is identical on a dry weight basis in every way to the therapeutic formulation except that it lacks the excipient compound. In embodiments, the viscosity-reducing amount is the amount of an excipient compound that reduces the viscosity of the formulation at least 30% when compared to the control formulation. In embodiments, the viscosity-reducing amount is the amount of an excipient compound that reduces the viscosity of the formulation at least 50% when compared to the control formulation. In embodiments, the viscosity-reducing amount is the amount of an excipient compound that reduces the viscosity of the formulation at least 70% when compared to the control formulation. In embodiments, the viscosity-reducing amount is the amount of an excipient compound that reduces the viscosity of the formulation at least 90% when compared to the control formulation.

In embodiments, the viscosity-reducing amount yields a therapeutic formulation having a viscosity of less than 100 cP. In other embodiments, the therapeutic formulation has a viscosity of less than 50 cP. In other embodiments, the therapeutic formulation has a viscosity of less than 20 cP. In yet other embodiments, the therapeutic formulation has a viscosity of less than 10 cP. The term “viscosity” as used herein refers to a dynamic viscosity value when measured by the methods disclosed herein.

Therapeutic formulations in accordance with this disclosure have certain advantageous properties that improve the formulation's stability. In embodiments, the therapeutic formulations are resistant to shear degradation, phase separation, clouding out, oxidation, deamidation, aggregation, precipitation, and denaturing. In embodiments, the therapeutic formulations are processed, purified, stored, syringed, dosed, filtered, and centrifuged more effectively, compared with a control formulation.

In embodiments, the therapeutic formulations are administered to a patient at high concentration of therapeutic protein. In embodiments, the therapeutic formulations are administered to patients in a smaller injection volume and/or with less discomfort than would be experienced with a similar formulation lacking the therapeutic excipient. In embodiments, the therapeutic formulations are administered to patients using a narrower gauge needle, or less syringe force that would be required with a similar formulation lacking the therapeutic excipient. In embodiments, the therapeutic formulations are administered as a depot injection. In embodiments, the therapeutic formulations extend the half-life of the therapeutic protein in the body.

These features of therapeutic formulations as disclosed herein would permit the administration of such formulations by intramuscular or subcutaneous injection in a clinical situation, i.e., a situation where patient acceptance of an intramuscular injection would include the use of small-bore needles typical for IM/SC purposes and the use of a tolerable (for example, 2-3 mL) injected volume, and where these conditions result in the administration of an effective amount of the formulation in a single injection at a single injection site. By contrast, injection of a comparable dosage amount of the therapeutic protein using conventional formulation techniques would be limited by the higher viscosity of the conventional formulation, so that a SC/M injection of the conventional formulation would not be suitable for a clinical situation.

Therapeutic formulations in accordance with this disclosure can have certain advantageous properties consistent with improved stability. In embodiments, the therapeutic formulations are resistant to shear degradation, phase separation, clouding out, precipitation, oxidation, deamidation, aggregation, and/or denaturing. In embodiments, the therapeutic formulations are processed, purified, stored, syringed, dosed, filtered, and/or centrifuged more effectively, compared with a control formulation.

In embodiments, the therapeutic formulations disclosed herein are resistant to monomer loss as measured by size exclusion chromatography (SEC) analysis. In SEC analysis, the main analyte peak is generally associated with the active component of the formulation, such as a therapeutic protein, and this main peak of the active component is referred to as the monomer peak. The monomer peak represents the amount of active component in the monomeric state, as opposed to aggregated (dimeric, trimeric, oligomeric, etc.). High concentration solutions of therapeutic proteins formulated with the excipient compounds described herein can be administered to patients using syringes or pre-filled syringes. Thus, the stability of a therapeutic formulation can be observed by the relative amount of monomer after an elapsed time. In embodiments, an improvement in stability of a therapeutic formulation as disclosed herein can be measured as a higher percent monomer after a certain elapsed time, as compared to the percent monomer in a control formulation that does not contain the excipient. In embodiments, an improvement in stability of a therapeutic formulation as disclosed herein can be measured as a higher percent monomer after exposure to a stress condition, as compared to the percent monomer in a control formulation that does not contain the excipient after exposure to the stress condition. In embodiments, the stress conditions can be a low temperature storage, high temperature storage, exposure to air, exposure to gas bubbles, exposure to shear conditions, or exposure to freeze/thaw cycles.

In embodiments, the therapeutic formulations of the invention are resistant to an increase in protein particle size as measured by dynamic light scattering (DLS) analysis. In DLS analysis, the particle size of the therapeutic protein can be observed as a median particle diameter. Ideally, the median particle diameter of the therapeutic protein should be relatively unchanged. An increase of the median particle diameter, therefore, can represent an aggregated protein. Thus, the stability of a therapeutic formulation can be observed by the relative change in median particle diameter after an elapsed time. In embodiments, the therapeutic formulations as disclosed herein are resistant to forming a polydisperse particle size distribution as measured by dynamic light scattering (DLS) analysis. In embodiments, an improvement in stability of a therapeutic formulation of the invention can be measured as a lower percent change of the median particle diameter after a certain elapsed time, as compared to the median particle diameter in a control formulation that does not contain the excipient. In embodiments, an improvement in stability of a therapeutic formulation as disclosed herein can be measured as a lower percent change of the median particle diameter after exposure to a stress condition, as compared to the percent change of the median particle diameter in a control formulation that does not contain the excipient. In other words, in embodiments, improved stability prevents an increase in particle size as measured by light scattering. In embodiments, the stress conditions can be a low temperature storage, high temperature storage, exposure to air, exposure to gas bubbles, exposure to shear conditions, or exposure to freeze/thaw cycles. In embodiments, an improvement in stability of a therapeutic formulation as disclosed herein can be measured as a less polydisperse particle size distribution as measured by DLS, as compared to the polydispersity of the particle size distribution in a control formulation that does not contain the excipient.

In embodiments, the therapeutic formulations as disclosed herein are resistant to precipitation as measured by turbidity, light scattering, or particle counting analysis. In embodiments, an improvement in stability of a therapeutic formulation as disclosed herein can be measured as a lower turbidity, lower light scattering, or lower particle count after a certain elapsed time, as compared to the turbidity, light scattering, or particle count values in a control formulation that does not contain the excipient. In embodiments, an improvement in stability of a therapeutic formulation as disclosed herein can be measured as a lower turbidity, lower light scattering, or lower particle count after exposure to a stress condition, as compared to the turbidity, light scattering, or particle count in a control formulation that does not contain the excipient. In embodiments, the stress conditions can be a low temperature storage, high temperature storage, exposure to air, exposure to gas bubbles, exposure to shear conditions, or exposure to freeze/thaw cycles.

In embodiments, the therapeutic excipient has antioxidant properties that stabilize the therapeutic protein against oxidative damage, thereby improving its stability. In embodiments, the therapeutic formulation is stored at ambient temperatures, or for extended time at refrigerator conditions without appreciable loss of potency of the therapeutic protein. In embodiments, the therapeutic formulation is dried down for storage until it is needed; then it is reconstituted with an appropriate solvent, e.g., water. Advantageously, the formulations prepared as described herein can be stable over a prolonged period of time, from months to years. When exceptionally long periods of storage are desired, the formulations can be preserved in a freezer (and later reactivated) without fear of protein denaturation. In embodiments, formulations can be prepared for long-term storage that do not require refrigeration.

In embodiments, the excipient compounds disclosed herein are added to the therapeutic formulation in a stability-improving amount. In embodiments, a stability-improving amount is the amount of an excipient compound that reduces the degradation of the formulation at least 10% when compared to a control formulation; for the purposes of this disclosure, a control formulation is a formulation containing the protein active ingredient that is substantially similar on a weight basis to the therapeutic formulation except that it lacks the excipient compound. In embodiments, the stability-improving amount is the amount of an excipient compound that reduces the degradation of the formulation at least 30% when compared to the control formulation. In embodiments, the stability-improving amount is the amount of an excipient compound that reduces the degradation of the formulation at least 50% when compared to the control formulation. In embodiments, the stability-improving amount is the amount of an excipient compound that reduces the degradation of the formulation at least 70% when compared to the control formulation. In embodiments, the stability-improving amount is the amount of an excipient compound that reduces the degradation of the formulation at least 90% when compared to the control formulation.

Methods for preparing therapeutic formulations may be familiar to skilled artisans. The therapeutic formulations of the present invention can be prepared, for example, by adding the excipient compound to the formulation before or after the therapeutic protein is added to the solution. The therapeutic formulation can, for example, be produced by combining the therapeutic protein and the excipient at a first (lower) concentration and then processed by filtration or centrifugation to produce a second (higher) concentration of the therapeutic protein. Therapeutic formulations can be made with one or more of the excipient compounds with chaotropes, kosmotropes, hydrotropes, and salts. Therapeutic formulations can be made with one or more of the excipient compounds using techniques such as encapsulation, dispersion, liposome, vesicle formation, and the like. Methods for preparing therapeutic formulations comprising the excipient compounds disclosed herein can include combinations of the excipient compounds. In embodiments, combinations of excipients can produce benefits in lower viscosity, improved stability, or reduced injection site pain. Other additives may be introduced into the therapeutic formulations during their manufacture, including preservatives, surfactants, sugars, sucrose, trehalose, polysaccharides, arginine, proline, hyaluronidase, stabilizers, buffers, and the like. As used herein, a pharmaceutically acceptable excipient compound is one that is non-toxic and suitable for animal and/or human administration.

In embodiments, therapeutic formulations of the present invention can be prepared by lyophilization. Lyophilization is a process by which water is removed from a liquid formulation by first freezing the liquid and the placing it under vacuum, allowing the ice to change directly from a solid to a vapor without passing through a liquid phase. The process of lyophilization includes three distinct but interdependent processes: freezing, primary drying (sublimation), and secondary drying (desorption). As the water or other liquid solvent freezes during the first step, the dissolved solid components of the therapeutic formulation remain in the residual liquid, forming a phase termed the freeze concentrate. The freeze concentrate solidifies within the crystalline lattice of ice as the freezing progresses. The ice is then removed by sublimation, carried out under vacuum at a temperature and pressure below the triple point value of the solution to enable conversion of the ice directly into the vapor state without passing through the liquid phase. After sublimation, there is still bound moisture present in the product. The third stage of lyophilization, desorption, involves raising the temperature at a low ambient pressure to drive off residual moisture.

Methods for preparing therapeutic formulations by lyophilization are familiar to skilled artisans. Such therapeutic formulations can comprise a protein and an excipient, where the excipient improves the stability of the protein, lowers the viscosity of the protein, improves the solubility of the protein, or a combination thereof. Excipients suitable for improving the stability or solubility of the therapeutic protein following its lyophilization and/or lowering the viscosity of the therapeutic protein following its lyophilization can be termed “lyo-enhancing excipients,” because their use enhances properties of the therapeutic formulation following its lyophilization and reconstitution. In embodiments, those excipients effective as lyo-enhancing excipients may have usefulness in improving the stability or reducing the viscosity of therapeutic formulations that have been subjected to lyophilization. In embodiments, a lyo-enhancing excipient is selected from the Excipient Compound Category I (Hindered Amines), as described below in more detail. As described below, one or more hindered amines, defined and detailed in the Excipient Compound Category I (Hindered Amines) below, can be added to a therapeutic formulation in order to improve its stability, viscosity, or both. Such excipients, when added to lyophilized formulations, can act as lyo-enhancing excipients. In embodiments, the lyo-enhancing excipient added to the therapeutic formulation is caffeine, with or without other hindered amines or other excipients being added simultaneously or sequentially. In certain aspects, the caffeine is added in an amount from about 2 mg/ml to about 100 mg/ml, about 5 mg/ml to about 100 mg/ml, about 10 mg/ml to about 50 mg/ml, about 10 mg/ml to about 40 mg/ml, and about 10 mg/ml to about 30 mg/ml. In yet additional aspects, the caffeine is added in an amount less than about 100 mg/ml, less than about 50 mg/ml, less than about 50 mg/ml, less than about 40 mg/ml, less than about 30 mg/ml, and less that about 25 mg/ml. In yet additional aspects, caffeine is added in an amount of about 2 to about 10 mg, or about 4 to about 8 mg, per 100 mg of therapeutic protein; optionally the therapeutic protein is a therapeutic antibody. In embodiments, caffeine is used as a lyo-enhancing excipient in combination with nicotinamide; in certain embodiments, nicotinamide can be used as a lyo-enhancing excipient with or without caffeine being present.

In embodiments, caffeine is present in the freeze concentrate or in the reconstituted formulation in amounts ranging from about 1 mM to about 100 mM, or in amounts ranging from about 10 mM to about 75 mM. In embodiments, nicotinamide is present in the freeze concentrate or in the reconstituted formulation in amounts ranging from about 1 mM to about 100 mM, or in amounts ranging from about 10 mM to about 80 mM. In embodiments, the formulations contain both caffeine and nicotinamide. In embodiments, the lyo-enhancing excipient is used in combination with conventional lyophilization excipients, such as sugars or polyols, polymers, lyoprotectants, buffers, inorganic salts, amino acids, chelating agents, and surfactants. In embodiments, the lyo-enhancing excipient is used in combination with conventional lyophilization excipients, such as trehalose, sucrose, mannitol, glucose, glycine, and polyvinylpyrrolidone. In embodiments, one or more lyo-enhancing excipients can be combined with the therapeutic protein in a solution that is buffered with histidine, phosphate, citrate, tris, succinate, or glutamate. In embodiments, one or more lyo-enhancing excipients can be combined with the therapeutic protein and a saccharide excipient such as trehalose, sucrose, mannitol, or glucose, where the saccharide to protein ratio is 0.1-10.0 on a mass basis. In embodiments, one or more lyo-enhancing excipients can be combined with the therapeutic protein and a saccharide excipient such as trehalose, sucrose, mannitol, or glucose, where the saccharide to protein ratio is 0.25-2.0 on a mass basis.

In embodiments, one or more lyo-enhancing excipients can be combined with the therapeutic protein before the freezing and drying steps are conducted, for example in amounts ranging from about 1 mM to about 100 mM. This way an excipient in the solution is treated by the various steps of lyophilization in tandem with the therapeutic protein, and it resides in the formulation at the conclusion of the process along with the lyophilized protein. The co-presence of the lyo-enhancing excipient and the therapeutic protein in the lyophilized formulation allows them to be re-dissolved approximately synchronously. In embodiments, the presence of the lyo-enhancing excipient improves the solubility of the lyophilized protein, wherein the lyophilized protein has a faster dissolution time and/or the reconstituted lyophilized protein contains less insoluble matter.

In embodiments, the therapeutic protein composition with lyo-enhancing excipients can be reconstituted to a high protein concentration, such as a protein concentration of at least about 50 mg/ml, at least about 100 mg/ml, or between about 50 mg/mL to about 300 mg/mL, or a protein concentration between about 100 mg/mL to about 250 mg/mL, or a protein concentration between about 125 mg/mL to about 200 mg/mL.

In embodiments, the therapeutic protein composition with lyo-enhancing excipients can be initially lyophilized and then reconstituted and fully dissolved in a reconstituting solvent in a short period of time, such as less than about 10 minutes, or about 0.5 to about 10 minutes. In embodiments, the therapeutic protein composition with lyo-enhancing excipients can be initially lyophilized and then reconstituted and fully dissolved in a reconstituting solvent in a short period of time, such as about 0.5 minutes to about 5 minutes. In embodiments, the therapeutic protein composition with lyo-enhancing excipients can be initially lyophilized and then reconstituted and fully dissolved in a reconstituting solvent in a short period of time, such as about 0.5 minutes to about 3 minutes.

In other embodiments, the therapeutic protein formulation is lyophilized using standard techniques, but in the absence of a lyo-enhancing excipient. Following lyophilization, the therapeutic protein is then reconstituted with a solvent (e.g., water, buffered solution, or other protein-specific formulation) that includes the lyo-enhancing excipient. In embodiments, a lyo-enhancing excipient is added via the reconstituting solvent at an amount such that the excipient level will be between about 0.01 and about 300 mg/mL in the reconstituted formulation. In embodiments, the lyo-enhancing excipient is added via the reconstituting solvent at an amount such that the excipient level will be between about 0.1 and about 200 mg/mL in the reconstituted formulation. In embodiments, the lyo-enhancing excipient is added via the reconstituting solvent at an amount such that the excipient level will be between about 1 and about 100 mg/mL in the reconstituted formulation. In embodiments, the lyo-enhancing excipient is added via the reconstituting solvent at an amount such that the excipient level will be between about 2 and about 50 mg/mL in the reconstituted formulation. In embodiments, the lyo-enhancing excipient is added via the reconstituting solvent at an amount such that the excipient level will be between about 1 and about 10% of the protein concentration on a mass basis in the reconstituted formulation. In embodiments, the lyo-enhancing excipient is added via the reconstituting solvent at an amount such that the excipient level will be between about 2 and about 9% of the protein concentration on a mass basis in the reconstituted formulation. In embodiments, the lyo-enhancing excipient is added via the reconstituting solvent at an amount such that the excipient level will be between about 3 and about 8% of the protein concentration on a mass basis in the reconstituted formulation.

In embodiments, the lyo-enhancing excipient is added to the lyophilized formulation in an amount that results in a reduced viscosity for the formulation following its reconstitution, as compared to a similar lyophilized formulation prepared and reconstituted in the absence of the lyo-enhancing excipient. In embodiments, the lyo-enhancing excipient is added to the lyophilized formulation in an amount that results in a reduction in insoluble protein in the formulation following its reconstitution, as compared to a similar lyophilized formulation prepared and reconstituted in the absence of the lyo-enhancing excipient.

In embodiments, a therapeutic formulation of infliximab in lyophilized form can comprise the following ingredients in the following ratios. For each 100 mg of infliximab, the lyophilized formulation includes:
a) Sucrose, 50-100 mg, or an equal amount of another sugar or polyol such as trehalose or sorbitol which can be substituted for sucrose;
b) Caffeine 2-10 mg, or 4-8 mg;
c) Optionally nicotinamide 1-10 mg;
d) A buffer, for example a phosphate or histidine buffer;
e) A surfactant, such as a polysorbate at 0.05-1 mg, or 0.1-0.5 mg;
f) Other optional ingredients such as bulking and crystalline agents such as mannitol and glycine, and other excipients such as amino acids.

In embodiments, a reconstituted lyophilized formulation can comprise the following ingredients:
a) Infliximab 100-200 mg/mL, or 125-175 mg/mL;
b) Sucrose 50-200 mg/mL, or preferably 90-120 mg/mL, or an equal amount of another sugar or polyol such as trehalose or sorbitol which can be substituted for sucrose;
c) Caffeine 25-100 mM, or 30-80 mM;
d) Optionally nicotinamide 10-100 mM or 25-50 mM;
e) Buffer, with a formulation pH between 5.0 and 7.5, or between 5.5 and 6.5;
f) A surfactant, such as polysorbate 50-500 ppm or 100-200 ppm.

Therapeutic formulations including lyophilized proteins and lyo-enhancing excipients can have an improved stability compared with lyophilized proteins in formulations that do not include the excipient. In embodiments, this stability improvement can be seen after aging of the lyophilized protein under storage conditions such as frozen (−80 to 0° C.), refrigerated (2 to 8° C.), controlled room temperature, elevated temperatures, body temperature, or ambient temperature. For the purposes of the invention, elevated temperatures are those between about 23° C. and about 50° C., and these conditions can be encountered during shipment and storage of the biologic. Body temperature is 35-39° C. and these temperatures can be important in cases where the biologic is stored in contact with the body such as a wearable device, or implanted into the body such as an implantable pump or device. In other embodiments, this stability improvement can be seen after aging a reconstituted solution of the lyophilized protein under storage conditions such as frozen (−80 to 0° C.), refrigerated (2 to 8° C.), controlled room temperature, elevated temperatures, body temperature, or ambient temperature.

Therapeutic formulations comprising lyophilized proteins and one or more lyo-enhancing excipients can possess desirable properties for use in treating patients. As an example, such a formulation comprising a lyophilized protein and a lyo-enhancing excipient can be used in a method of treating a patient, where the excipient improves the stability of the protein, reduces the insoluble particle count of the protein solution, lowers the viscosity of the protein solution, or a combination thereof. Such a formulation can offer advantages in treating patients in situations where the lyo-enhancing excipient improves the safety of the drug, or reduces the incidence of immune reactions to the administration of the drug. Such a formulation can offer advantages in treating patients in situations where the lyo-enhancing excipient reduces the viscosity of the reconstituted protein solution, thereby improving the ease of administration for the patient or caregiver or permitting the delivery of a more concentrated protein dose to the patient at a more convenient dosage schedule.

While the use of a lyo-enhancing excipient has been described above for use in processing before lyophilization is commenced, and after lyophilization has been completed, in embodiments the lyo-enhancing excipient can be added to the lyophilized product, producing an improved lyophilized formulation that dissolves faster and/or yields lower viscosity. In other embodiments, a lyo-enhancing excipient can be added to the therapeutic protein formulation before lyophilization, and the same or a different lyo-enhancing excipient can be included in the reconstitution solvent to be added to the lyophilized formulation when it is reconstituted. In other words, one or more lyo-enhancing excipients can be added to a therapeutic protein formulation both before it is lyophilized and after lyophilization when it is being reconstituted. The use of the lyo-enhancing excipients can act synergistically to improve the stability and/or viscosity of the final reconstituted formulation. In embodiments, the lyo-enhancing excipient used before lyophilization can be the same as the lyo-enhancing excipient added to the reconstitution solvent; in other embodiments, the lyo-enhancing excipients can be different. In embodiments, one or more lyo-enhancing excipients can be used in the formulation before lyophilization, which can be the same as or different than the one or more lyo-enhancing excipients that are used in the reconstitution solvent.

### 4. Non-Therapeutic Formulations

In one aspect, the formulations and methods disclosed herein provide stable liquid formulations of improved or reduced viscosity, comprising a non-therapeutic protein in an effective amount and an excipient compound. In embodiments, the formulation improves the stability while providing an acceptable concentration of active ingredients and an acceptable viscosity. In embodiments, the formulation provides an improvement in stability when compared to a control formulation; for the purposes of this disclosure, a control formulation is a formulation containing the protein active ingredient that is identical on a dry weight basis in every way to the non-therapeutic formulation except that it lacks the excipient compound.

It is understood that the viscosity of a liquid protein formulation can be affected by a variety of factors, including but not limited to: the nature of the protein itself (e.g., enzyme, structural protein, degree of hydrolysis, etc.); its size, three-dimensional structure, chemical composition, and molecular weight; its concentration in the formulation; the components of the formulation besides the protein; the desired pH range; and the storage conditions for the formulation.

In embodiments, the non-therapeutic formulation contains at least 25 mg/mL of protein active ingredient. In other embodiments, the non-therapeutic formulation contains at least 100 mg/mL of protein active ingredient. In other embodiments, the non-therapeutic formulation contains at least 200 mg/mL of protein active ingredient. In yet other embodiments, the non-therapeutic formulation solution contains at least 300 mg/mL of protein active ingredient. Generally, the excipient compounds disclosed herein are added to the non-therapeutic formulation in an amount between about 5 to about 300 mg/mL. In embodiments, the excipient compound is added in an amount of about 10 to about 200 mg/mL. In embodiments, the excipient compound is added in an amount of about 20 to about 100 mg/mL. In embodiments, the excipient is added in an amount of about 25 to about 75 mg/mL.

Excipient compounds of various molecular weights are selected for specific advantageous properties when combined with the protein active ingredient in a formulation. Examples of non-therapeutic formulations comprising excipient compounds are provided below. In embodiments, the excipient compound has a molecular weight of <5000 Da. In embodiments, the excipient compound has a molecular weight of <1000 Da. In embodiments, the excipient compound has a molecular weight of <500 Da.

In embodiments, the excipient compounds disclosed herein are added to the non-therapeutic formulation in a viscosity-reducing amount. In embodiments, a viscosity-reducing amount is the amount of an excipient compound that reduces the viscosity of the formulation at least 10% when compared to a control formulation; for the purposes of this disclosure, a control formulation is a formulation containing the protein active ingredient that is identical on a dry weight basis in every way to the therapeutic formulation except that it lacks the excipient compound. In embodiments, the viscosity-reducing amount is the amount of an excipient compound that reduces the viscosity of the formulation at least 30% when compared to the control formulation. In embodiments, the viscosity-reducing amount is the amount of an excipient compound that reduces the viscosity of the formulation at least 50% when compared to the control formulation. In embodiments, the viscosity-reducing amount is the amount of an excipient compound that reduces the viscosity of the formulation at least 70% when compared to the control formulation. In embodiments, the viscosity-reducing amount is the amount of an excipient compound that reduces the viscosity of the formulation at least 90% when compared to the control formulation.

In embodiments, the viscosity-reducing amount yields a non-therapeutic formulation having a viscosity of less than 100 cP. In other embodiments, the non-therapeutic formulation has a viscosity of less than 50 cP. In other embodiments, the non-therapeutic formulation has a viscosity of less than 20 cP. In yet other embodiments, the non-therapeutic formulation has a viscosity of less than 10 cP. The term “viscosity” as used herein refers to a dynamic viscosity value.

Non-therapeutic formulations in accordance with this disclosure can have certain advantageous properties that improve the formulation's stability. In embodiments, the non-therapeutic formulations are resistant to shear degradation, phase separation, clouding out, oxidation, deamidation, aggregation, precipitation, and denaturing. In embodiments, the therapeutic formulations can be processed, purified, stored, pumped, filtered, and centrifuged more effectively, compared with a control formulation.

In embodiments, the non-therapeutic excipient has antioxidant properties that stabilize the non-therapeutic protein against oxidative damage, thereby improving its stability. In embodiments, the non-therapeutic formulation is stored at ambient temperatures, or for extended time at refrigerator conditions without appreciable loss of potency for the non-therapeutic protein. In embodiments, the non-therapeutic formulation is dried down for storage until it is needed; then it can be reconstituted with an appropriate solvent, e.g., water. Advantageously, the formulations prepared as described herein is stable over a prolonged period of time, from months to years. When exceptionally long periods of storage are desired, the formulations are preserved in a freezer (and later reactivated) without fear of protein denaturation. In embodiments, formulations are prepared for long-term storage that do not require refrigeration.

In embodiments, the excipient compounds disclosed herein are added to the non-therapeutic formulation in a stability-improving amount. In embodiments, a stability-improving amount is the amount of an excipient compound that reduces the degradation of the formulation at least 10% when compared to a control formulation; for the purposes of this disclosure, a control formulation is a formulation containing the protein active ingredient that is substantially similar on a dry weight basis to the therapeutic formulation except that it lacks the excipient compound. In embodiments, the stability-improving amount is the amount of an excipient compound that reduces the degradation of the formulation at least 30% when compared to the control formulation. In embodiments, the stability-improving amount is the amount of an excipient compound that reduces the degradation of the formulation at least 50% when compared to the control formulation. In embodiments, the stability-improving amount is the amount of an excipient compound that reduces the degradation of the formulation at least 70% when compared to the control formulation. In embodiments, the stability-improving amount is the amount of an excipient compound that reduces the degradation of the formulation at least 90% when compared to the control formulation.

Methods for preparing non-therapeutic formulations comprising the excipient compounds disclosed herein may be familiar to skilled artisans. For example, the excipient compound can be added to the formulation before or after the non-therapeutic protein is added to the solution. The non-therapeutic formulation can be produced at a first (lower) concentration and then processed by filtration or centrifugation to produce a second (higher) concentration. Non-therapeutic formulations can be made with one or more of the excipient compounds with chaotropes, kosmotropes, hydrotropes, and salts. Non-therapeutic formulations can be made with one or more of the excipient compounds using techniques such as encapsulation, dispersion, liposome, vesicle formation, and the like. Other additives can be introduced into the non-therapeutic formulations during their manufacture, including preservatives, surfactants, stabilizers, and the like.

### 5. Excipient Compounds

Several excipient compounds are described herein, each suitable for use with one or more therapeutic or non-therapeutic proteins, and each allowing the formulation to be composed so that it contains the protein(s) at a high concentration. Some of the categories of excipient compounds described below are: (1) hindered amines; (2) anionic aromatics; (3) functionalized amino acids; (4) oligopeptides; (5) short-chain organic acids; (6) low-molecular-weight aliphatic polyacids; (7) diones and sulfones; (8) zwitterionic excipients; and (9) crowding agents with hydrogen bonding elements. Without being bound by theory, the excipient compounds described herein are thought to associate with certain fragments, sequences, structures, or sections of a therapeutic protein that otherwise would be involved in inter-particle (i.e., protein-protein) interactions. The association of these excipient compounds with the therapeutic or non-therapeutic protein can mask the inter-protein interactions such that the proteins can be formulated in high concentration without causing excessive solution viscosity. In embodiments, the excipient compound can result in more stable protein-protein interaction; protein-protein interaction can be measured by the protein diffusion parameter kD, or the osmotic second virial coefficient B22, or by other techniques familiar to skilled artisans.

Excipient compounds advantageously can be water-soluble, therefore suitable for use with aqueous vehicles. In embodiments, the excipient compounds have a water solubility of >1 mg/mL. In embodiments, the excipient compounds have a water solubility of >10 mg/mL. In embodiments, the excipient compounds have a water solubility of >100 mg/mL. In embodiments, the excipient compounds have a water solubility of >500 mg/mL. In embodiments, cosolutes or hydrotropes can be added in combination with the excipient compounds to increase the solubility of the excipient compounds. For example, certain excipients may have limited solubility in the aqueous solution containing the therapeutic protein, and this solubility can be even lower at cold storage conditions. Cosolutes or hydrotropes can be added to increase the solubility of the excipients in solution at cold storage conditions or at ambient room temperature or elevated temperature conditions. Examples of the cosolutes and hydrotropes include benzoate salts, benzyl alcohol, phenylalanine, nicotinamide, proline, procaine, 2,5-dihydroxybenzoate, tyramine, and saccharin. Advantageously for therapeutic proteins, the excipient compounds can be derived from materials that are biologically acceptable and are non-immunogenic and are thus suitable for pharmaceutical use. In therapeutic embodiments, the excipient compounds can be metabolized in the body to yield biologically compatible and non-immunogenic byproducts.

a. Excipient Compound Category 1: Hindered Amines

Solutions of therapeutic or non-therapeutic proteins can be formulated with hindered amine small molecules as excipient compounds. As used herein, the term “hindered amine” refers to a small molecule containing at least one bulky or sterically hindered group, consistent with the examples below. Hindered amines can be used in the free base form, in the protonated form, or a combination of the two. In protonated forms, the hindered amines can be associated with an anionic counterion such as chloride, hydroxide, bromide, iodide, fluoride, acetate, formate, phosphate, sulfate, or carboxylate. Hindered amine compounds useful as excipient compounds can contain secondary amine, tertiary amine, quaternary ammonium, pyridinium, pyrrolidone, pyrrolidine, piperidine, morpholine, or guanidinium groups, such that the excipient compound has a cationic charge in aqueous solution at neutral pH. The hindered amine compounds also contain at least one bulky or sterically hindered group, such as cyclic aromatic, cycloaliphatic, cyclohexyl, or alkyl groups. In embodiments, the sterically hindered group can itself be an amine group such as a dialkylamine, trialkylamine, guanidinium, pyridinium, or quaternary ammonium group. Without being bound by theory, the hindered amine compounds are thought to associate with aromatic sections of the proteins such as phenylalanine, tryptophan, and tyrosine, by a cation pi interaction. In embodiments, the cationic group of the hindered amine can have an affinity for the electron rich pi structure of the aromatic amino acid residues in the protein, so that they can shield these sections of the protein, thereby decreasing the tendency of such shielded proteins to associate and aggregate.

In embodiments, the hindered amine excipient compounds has a chemical structure comprising imidazole, imidazoline, or imidazolidine groups, or salts thereof, such as imidazole, 1-methylimidazole, 4-methylimidazole, 1-hexyl-3-methylimidazolium chloride, 1,3-Dimethyl-2-imidazolidinone, histamine, 4-methylhistamine, alpha-methylhistamine, betahistine, beta-alanine, 2-methyl-2-imidazoline, 1-butyl-3-methylimidazolium chloride, uric acid, potassium urate, betazole, carnosine, aspartame, saccharin, acesulfame potassium, xanthine, theophylline, theobromine, caffeine, and anserine. In embodiments, the hindered amine excipient compounds is selected from the group consisting of dimethylethanolamine, dimethylaminopropylamine, triethanolamine, dimethylbenzylamine, dimethylcyclohexylamine, diethylcyclohexylamine, dicyclohexylmethylamine, hexamethylene biguanide, poly(hexamethylene biguanide), imidazole, lysine, methylglycine, sarcosine, dimethylglycine, agmatine, diazabicyclo[2.2.2]octane, folinic acid sodium salt, folinic acid calcium salt, tetramethylethylenediamine, N,N-dimethylethanolamine, ethanolamine phosphate, glucosamine, choline chloride, phosphocholine, niacinamide, isonicotinamide, N,N-diethyl nicotinamide, nicotinic acid sodium salt, isonicotinic acid salts, tyramine, 3-aminopyridine, 2,4,6-trimethylpyridine, 3-pyridine methanol, nicotinamide adenine dinucleotide, biotin, morpholine, N-methylpyrrolidone, 2-pyrrolidinone, dipyridamole, procaine, lidocaine, dicyandiamide-taurine adduct, 2-pyridylethylamine, 6-hydroxypyridine-2-carboxylic acid, dicyandiamide-benzyl amine adduct, dicyandiamide-alkylamine adduct, dicyandiamide-cycloalkylamine adduct, and dicyandiamide-aminomethanephosphonic acid adducts. In embodiments, the hindered amine excipient compounds is selected from the group consisting of 1-(1-adamantyl) ethylamine, 1-aminobenzotriazole, 2-dimethylaminoethanol, 2-methyl-2-imidazoline, 2-methylimidazole, 3-aminobenzamide, 3-indoleacetic acid, 4-aminopyridine, 6-amino-1,3-dimethyluracil, acetylcholine, agmatine sulfate, benzalkonium chloride, ethyl 3-aminobenzoate, sulfacetamide, butyl anthranilate, amino hippuric acid, benzamide oxime, benzethonium chloride, benzylamine, berberine chloride, castanospermine, clemizole, cycloserine, phenylserine, DL-3-phenylserine, cysteamine, cytidine, diethanolamine, diphenhydramine, DL-norepinephrine, dopamine, emtricitabine, ethanolamine, guanfacine, isonicotinamide, lithium chloride, meglumine, methyl cytidine, myristyl gamma picolinium chloride, niacinamide, phenylethylamine, polyethyleneimine, pyridoxine, rasagiline mesylate, serotonin, synephrine, neamine, spermine, spermidine, 1,3-diaminopropane, adenosine, chloroquine phosphate, cystamine, pyridyl ethylamine, tetramethylethylenediamine, tryptamine, tyramine, 1-methylimidazole, spectinomycin, cyclohexane methylamine, N,N-dimethylphenethylamine, phenethylamine, tetraethylammonium, tetramethyl ammonium acetate, dicyclomine, hordenine, methylaminoethylpyridine, nicotinamide riboside, 1-butylimidazole, 1-hexylimidazole, 1-methylimidazole, 2-ethylimidazole, 2-n-butylimidazole, 2-methylimidazole, 1-dodecylimidazole, other imidazoles alkylated at the 1 or 2 position with a Ci to Cu hydrocarbon chain, pridinol methanesulfonate, hemin, N,N-dimethylphenethylamine, voglibose, N-ethyl-L-glutamine, nicotine, piperazine, demeclocycline, and the salts thereof. In embodiments, the hindered amine excipient compounds can have a phenethylamine functional group, such as phenethylamine, diphenhydramine, N-methylphenethylamine, N,N-dimethylphenethylamine, β,3-dihydroxyphenethylamine, β,3-dihydroxy-N-methylphenethylamine, 3-hydroxyphenethylamine, 4-hydroxyphenethylamine, tyrosinol, tyramine, N-methyltyramine, and hordenine. Preferably the phenethylamine containing structure is a non-psychoactive compound.

Suitable salts of the hindered amine structures can be chloride, bromide, acetate, citrate, sulfate, and phosphate. In embodiments, a hindered amine compound consistent with this disclosure is formulated as a protonated ammonium salt. In embodiments, a hindered amine compound consistent with this disclosure is formulated as a salt with an inorganic anion or organic anion as the counterion.

In embodiments, high concentration solutions of therapeutic or non-therapeutic proteins are formulated with a combination of caffeine with a benzoic acid, a hydroxybenzoic acid, or a benzenesulfonic acid as excipient compounds. In embodiments, the hindered amine excipient compounds are metabolized in the body to yield biologically compatible byproducts. In some embodiments, the hindered amine excipient compound is present in the formulation at a concentration of about 250 mg/mL or less. In additional embodiments, the hindered amine excipient compound is present in the formulation at a concentration of about 10 mg/mL to about 200 mg/mL. In yet additional aspects, the hindered amine excipient compound is present in the formulation at a concentration of about 20 to about 120 mg/mL.

In embodiments, viscosity-reducing excipients in this hindered amine category may include methylxanthines such as caffeine and theophylline, although their use has typically been limited due to their low water solubility. In some applications it may be advantageous to have higher concentrated solutions of these viscosity-reducing excipients despite their low water solubility. For example, in processing it may be advantageous to have a concentrated excipient solution that can be added to a concentrated protein solution so that adding the excipient does not dilute the protein below the desired final concentration. In other cases, despite its low water solubility, additional viscosity-reducing excipient may be necessary to achieve the desired viscosity reduction, stability, tonicity etc. of a final protein formulation. In embodiments, a highly concentrated excipient solution may be formulated (i) as a viscosity-reducing excipient at a concentration 1.5 to 50 times higher than the effective viscosity-reducing amount, or (ii) as a viscosity-reducing excipient at a concentration 1.5 to 50 times higher than its literature reported solubility in pure water at 298 K (e.g., as reported in The Merck Index; Royal Society of Chemistry; Fifteenth Edition, (Apr. 30, 2013)), or both.

Certain co-solutes have been found to substantially increase the solubility limit of these low solubility viscosity-reducing excipients, allowing for excipient solutions at concentrations multiple times higher than literature reported solubility values. These co-solutes can be classified under the general category of hydrotropes. Co-solutes found to provide the greatest improvement in solubility for this application were generally highly soluble in water (>0.25 M) at ambient temperature and physiological pH, and contained either a pyridine or benzene ring. Examples of compounds that may be useful as co-solutes include aniline HCl, isoniacinamide, niacinamide, n-methyltyramine HCl, phenol, procaine HCl, resorcinol, saccharin calcium salt, saccharin sodium salt, sodium aminobenzoic acid, sodium benzoate, sodium parahydroxybenzoate, sodium metahydroxybenzoate, sodium 2,5-dihydroxybenzoate, sodium salicylate, sodium sulfanilate, sodium parahydroxybenzene sulfonate, synephrine, and tyramine HCl.

In embodiments, certain hindered amine excipient compounds can possess other pharmacological properties. As examples, xanthines are a category of hindered amines commonly having independent pharmacological properties, including stimulant properties and bronchodilator properties when systemically absorbed. Representative xanthines include caffeine, aminophylline, 3-isobutyl-1-methylxanthine, paraxanthine, pentoxifylline, theobromine, theophylline, and the like. Methylated xanthines are understood to affect force of cardiac contraction, heart rate, and bronchodilation. In some embodiments, the xanthine excipient compound is present in the formulation at a concentration of about 30 mg/mL or less.

Another category of hindered amines having independent pharmacological properties are the local injectable anesthetic compounds. Local injectable anesthetic compounds are hindered amines that have a three-component molecular structure of (a) a lipophilic aromatic ring, (b) an intermediate ester or amide linkage, and (c) a secondary or tertiary amine. This category of hindered amines is understood to interrupt neural conduction by inhibiting the influx of sodium ions, thereby inducing local anesthesia. The lipophilic aromatic ring for a local anesthetic compound may be formed of carbon atoms (e.g., a benzene ring) or it may comprise heteroatoms (e.g., a thiophene ring). Representative local injectable anesthetic compounds include, but are not limited to, amylocaine, articaine, bupivicaine, butacaine, butanilicaine, chlorprocaine, cocaine, cyclomethycaine, dimethocaine, editocaine, hexylcaine, isobucaine, levobupivacaine, lidocaine, metabutethamine, metabutoxycaine, mepivacaine, meprylcaine, propoxycaine, prilocaine, procaine, piperocaine, tetracaine, trimecaine, and the like. The local injectable anesthetic compounds can have multiple benefits in protein therapeutic formulations, such as reduced viscosity, improved stability, and reduced pain upon injection. In some embodiments, the local anesthetic compound is present in the formulation in a concentration of about 50 mg/mL or less.

In embodiments, a hindered amine having independent pharmacological properties is used as an excipient compound in accordance with the formulations and methods described herein. In some embodiments, the excipient compounds possessing independent pharmacological properties are present in an amount that does not have a pharmacological effect and/or that is not therapeutically effective. In other embodiments, the excipient compounds possessing independent pharmacological properties are present in an amount that does have a pharmacological effect and/or that is therapeutically effective. In certain embodiments, a hindered amine having independent pharmacological properties is used in combination with another excipient compound that has been selected to decrease formulation viscosity, where the hindered amine having independent pharmacological properties is used to impart the benefits of its pharmacological activity. For example, a local injectable anesthetic compound can be used to decrease formulation viscosity and also to reduce pain upon injection of the formulation. The reduction of injection pain can be caused by anesthetic properties; also, a lower injection force can be required when the viscosity is reduced by the excipients. Alternatively, a local injectable anesthetic compound can be used to impart the desirable pharmacological benefit of decreased local sensation during formulation injection, while being combined with another excipient compound that reduces the viscosity of the formulation.

b. Excipient Compound Category 2: Anionic Aromatics

Solutions of therapeutic or non-therapeutic proteins can be formulated with anionic aromatic small molecule compounds as excipient compounds. The anionic aromatic excipient compounds can contain an aromatic functional group such as phenyl, benzyl, aryl, alkylbenzyl, hydroxybenzyl, phenolic, hydroxyaryl, heteroaromatic group, or a fused aromatic group. The anionic aromatic excipient compounds also can contain an anionic functional group such as carboxylate, oxide, phenoxide, sulfonate, sulfate, phosphonate, phosphate, or sulfide. While the anionic aromatic excipients might be described as an acid, a sodium salt, or other, it is understood that the excipient can be used in a variety of salt forms. Without being bound by theory, an anionic aromatic excipient compound is thought to be a bulky, sterically hindered molecule that can associate with cationic segments of a protein, so that they can shield these sections of the protein, thereby decreasing the interactions between protein molecules that render the protein-containing formulation viscous or result in stability problems.

In embodiments, examples of anionic aromatic excipient compounds include compounds such as salicylic acid, aminosalicylic acid, hydroxybenzoic acid, aminobenzoic acid, para-aminobenzoic acid, benzenesulfonic acid, hydroxybenzenesulfonic acid, 4-phenylbutyric acid, naphthalenesulfonic acid, 1,5-naphthalenedisulfonic acid, 2,6-naphthalenedisulfonic acid, 2,7-naphthalenedisulfonic acid, hydroquinone sulfonic acid, sulfanilic acid, vanillic acid, homovanillic acid, vanillin, vanillin-taurine adduct, aminophenol, anthranilic acid, cinnamic acid, menadione sodium bisulfite, 4-hydroxy-3-methoxycinnamic acid, caffeic acid, chlorogenic acid, gentisic acid, coumaric acid, adenosine monophosphate, indole acetic acid, potassium urate, furan dicarboxylic acid, furan-2-acrylic acid, 2-furanpropionic acid, sodium phenylpyruvate, sodium hydroxyphenylpyruvate, trimethoxybenzoic acid, dihydroxybenzoic acid, ferrocenecarboxylic acid, trihydroxybenzoic acid, pyrogallol, benzoic acid, and the salts of the foregoing acids. In embodiments, the anionic aromatic excipient compounds are formulated in the ionized salt form. In embodiments, an anionic aromatic compound is formulated as the salt of a hindered amine, such as dimethylcyclohexylammonium hydroxybenzoate. In embodiments, the anionic aromatic excipient compounds are formulated with various counterions such as organic cations. In embodiments, high concentration solutions of therapeutic or non-therapeutic proteins are formulated with anionic aromatic excipient compounds and caffeine. In embodiments, the anionic aromatic excipient compounds are metabolized in the body to yield biologically compatible byproducts.

In embodiments, examples of aromatic excipient compounds include phenols and polyphenols. As used herein, the term “phenol” refers an organic molecule that consists of at least one aromatic group or fused aromatic group bonded to at least one hydroxyl group and the term “polyphenol” refers to an organic molecule that consists of more than one phenol group. Such excipients can be advantageous under certain circumstances, for example when used in formulations with high concentration solutions of therapeutic or nontherapeutic PEGylated proteins to lower solution viscosity. Non-limiting examples of phenols include the benzenediols resorcinol (1,3-benzenediol), catechol (1,2-benzenediol) and hydroquinone (1,4-benzenediol), the benzenetriols hydroxyquinol (1,2,4-benzenetriol), pyrogallol (1,2,3-benzenetriol), and phloroglucinol (1,3,5-benzenetriol), the benzenetetrols 1,2,3,4-Benzenetetrol and 1,2,3,5-Benzenetetrol, and benzenepentol and benzenehexol. Non-limiting examples of polyphenols include tannic acid, ellagic acid, epigallocatechin gallate, catechin, tannins, ellagitannins, and gallotannins. More generally, phenolic and polyphenolic compounds include, but are not limited to, flavonoids, lignans, phenolic acids, and stilbenes. Flavonoid compounds include, but are not limited to, anthocyanins, chalcones, dihydrochalcones, dihydroflavanols, flavanols, flavanones, flavones, flavonols, and isoflavonoids. Phenolic acids include, but are not limited to, hydroxybenzoic acids, hydroxycinnamic acids, hydroxyphenylacetic acids, hydroxyphenylpropanoic acids, and hydroxyphenylpentanoic acids. Other polyphenolic compounds include, but are not limited to, alkylmethoxyphenols, alkylphenols, curcuminoids, hydroxybenzaldehydes, hydroxybenzoketones, hydroxycinnamaldehydes, hydroxycoumarins, hydroxyphenylpropenes, methoxyphenols, naphtoquinones, hydroquinones, phenolic terpenes, resveratrol, and tyrosols. In embodiments, the polyphenol is tannic acid. In embodiments, the phenol is gallic acid. In embodiments, the phenol is pyrogallol. In embodiments, the phenol is resorcinol. Without being bound by theory, the hydroxyl groups of phenolic compounds, e.g., gallic acid, pyrogallol, and resorcinol, form hydrogen bonds with ether oxygen atoms in the backbone of the PEG chain and thus form a phenol/PEG complex that fundamentally alters the PEG solution structure such that the solution viscosity is reduced. Polyphenolic compounds, such as tannic acid, derive their viscosity-reducing properties from their respective phenol group building blocks, such as gallic acid, pyrogallol, and resorcinol. The specific organization of the phenol groups within a polyphenolic compound can give rise to complex behavior in which a viscosity reduction attained by the addition of a phenol is enhanced by the addition of a lower quantity of the respective polyphenol.

c. Excipient Compound Category 3: Functionalized Amino Acids

Solutions of therapeutic or non-therapeutic proteins can be formulated with one or more functionalized amino acids, where a single functionalized amino acid or an oligopeptide comprising one or more functionalized amino acids may be used as the excipient compound. In embodiments, the functionalized amino acid compounds comprise molecules (“amino acid precursors”) that can be hydrolyzed or metabolized to yield amino acids. In embodiments, the functionalized amino acids can contain an aromatic functional group such as phenyl, benzyl, aryl, alkylbenzyl, hydroxybenzyl, hydroxyaryl, heteroaromatic group, or a fused aromatic group. In embodiments, the functionalized amino acid compounds can contain esterified amino acids, such as methyl, ethyl, propyl, butyl, benzyl, cycloalkyl, glyceryl, hydroxyethyl, hydroxypropyl, PEG, and PPG esters. In embodiments, the functionalized amino acid compounds are selected from the group consisting of arginine ethyl ester, arginine methyl ester, arginine hydroxyethyl ester, and arginine hydroxypropyl ester. In embodiments, the functionalized amino acid compound is a charged ionic compound in aqueous solution at neutral pH. For example, a single amino acid can be derivatized by forming an ester, like an acetate or a benzoate, and the hydrolysis products would be acetic acid or benzoic acid, both natural materials, plus the amino acid. In embodiments, the functionalized amino acid excipient compounds are metabolized in the body to yield biologically compatible byproducts.

d. Excipient Compound Category 4: Oligopeptides

Solutions of therapeutic or non-therapeutic proteins can be formulated with oligopeptides as excipient compounds. In embodiments, the oligopeptide is designed such that the structure has a charged section and a bulky section. In embodiments, the oligopeptides consist of between 2 and 10 peptide subunits. The oligopeptide can be bi-functional, for example a cationic amino acid coupled to a non-polar one, or an anionic one coupled to a non-polar one. In embodiments, the oligopeptides consist of between 2 and 5 peptide subunits. In embodiments, the oligopeptides are homopeptides such as polyglutamic acid, polyaspartic acid, poly-lysine, poly-arginine, and poly-histidine. In embodiments, the oligopeptides have a net cationic charge. In other embodiments, the oligopeptides are heteropeptides, such as Trp2Lys3. In embodiments, the oligopeptide can have an alternating structure such as an ABA repeating pattern. In embodiments, the oligopeptide can contain both anionic and cationic amino acids, for example, Arg-Glu, Lys-Glu, His-Glu, Arg-Asp, Lys-Asp, His-Asp, Glu-Arg, Glu-Lys, Glu-His, Asp-Arg, Asp-Lys, and Asp-His. Without being bound by theory, the oligopeptides comprise structures that can associate with proteins in such a way that it reduces the intermolecular interactions that lead to high viscosity solutions and stability problems; for example, the oligopeptide-protein association can be a charge-charge interaction, leaving a somewhat non-polar amino acid to disrupt hydrogen bonding of the hydration layer around the protein, thus lowering viscosity or improving stability. In some embodiments, the oligopeptide excipient is present in the composition in a concentration of about 50 mg/mL or less.

e. Excipient Compound Category 5: Short-Chain Organic Acids

Solutions of therapeutic or non-therapeutic proteins can be formulated with short-chain organic acids as excipient compounds. As used herein, the term “short-chain organic acids” refers to C₂-C₆ organic acid compounds and the salts, esters, amides, or lactones thereof. This category includes saturated and unsaturated carboxylic acids, hydroxy functionalized carboxylic acids, amides, and linear, branched, or cyclic carboxylic acids. In embodiments, the acid group in the short-chain organic acid is a carboxylic acid, sulfonic acid, phosphonic acid, or a salt thereof.

Solutions of therapeutic or non-therapeutic proteins can be formulated with short-chain organic acids, for example, the acid or salt forms of sorbic acid, valeric acid, propionic acid, caproic acid, and ascorbic acid as excipient compounds. Examples of excipient compounds in this category include potassium sorbate, calcium gluconate, glucuronic acid, calcium lactate, 2-hydroxylactate, sodium glycolate, potassium glycolate, ammonium glycolate, sodium valproate, taurine, acetohydroxamic acid, acetone sodium bisulfite adduct, acetyl hydroxyproline, calcium propionate, magnesium propionate, sodium propionate, sodium ascorbate, and salts thereof.

f. Excipient Compound Category 6: Low Molecular Weight Polyacids

Solutions of therapeutic or non-therapeutic proteins or PEGylated proteins can be formulated with certain excipient compounds that enable lower solution viscosity or improved stability, where such excipient compounds are low molecular weight polyacids. Low molecular weight polyacids can include organic polyacids or inorganic polyacids. These low molecular weight polyacid excipients can also be used in combination with other excipients.

Organic polyacids, in embodiments, can be structured as low molecular weight aliphatic polyacids. As used herein, the term “low molecular weight aliphatic polyacids” refers to organic aliphatic polyacids having a molecular weight less than about 1500 Da, and having at least two acidic groups, where an acidic group is understood to be a proton-donating moiety. Non-limiting examples of acidic groups include carboxylate, phosphonate, phosphate, sulfonate, sulfate, nitrate, and nitrite groups. Acidic groups on the low molecular weight aliphatic polyacid can be in the anionic salt form such as carboxylate, phosphonate, phosphate, sulfonate, sulfate, nitrate, and nitrite; their counterions can be sodium, potassium, lithium, and ammonium. Specific examples of low molecular weight aliphatic polyacids useful for interacting with PEGylated proteins as described herein include oxalic acid, adipic acid, pimelic acid, suberic acid, azelaic acid, sebacic acid, succinic acid, maleic acid, tartaric acid, glutaric acid, malonic acid, itaconic acid, methyl malonic acid, azelaic acid, citric acid, 3,6,9-trioxaundecanedioic acid, ethylenediaminetetraacetic acid (EDTA), aspartic acid, pyrrolidone carboxylic acid, pyroglutamic acid, glutamic acid, alendronic acid, medronic acid, etidronic acid and salts thereof.

In other embodiments, low molecular weight polyacids are inorganic polyacids. Further examples of low molecular weight polyacids in their anionic salt form include phosphate (PO₄³⁻), hydrogen phosphate (HPO₄²⁻), dihydrogen phosphate (H₂PO₄⁻), sulfate, bisulfate (HSO₄⁻), pyrophosphate (P₂O₇⁴⁻), hexametaphosphate, borate, carbonate (CO₃²⁻), and bicarbonate (HCO₃⁻). The counterion for the anionic salts can be Na, Li, K, or ammonium ion.

In embodiments, the low molecular weight aliphatic polyacid can also be an alpha hydroxy acid, where there is a hydroxyl group adjacent to a first acidic group, for example glycolic acid, lactic acid, and gluconic acid and salts thereof. In embodiments, the low molecular weight aliphatic polyacid is an oligomeric form that bears more than two acidic groups, for example polyacrylic acid, polyphosphates, polypeptides and salts thereof. In some embodiments, the low molecular weight aliphatic polyacid excipient is present in the composition in a concentration of about 50 mg/mL or less.

g. Excipient Compound Category 7: Diones and Sulfones

An effective viscosity-reducing or stabilizing excipient can be a molecule containing a sulfone, sulfonamide, or dione functional group that is soluble in pure water to at least 1 g/L at 298K and that has a net neutral charge at pH 7. Preferably, the molecule has a molecular weight of less than 1000 g/mol and more preferably less than 500 g/mol. The diones and sulfones effective in reducing viscosity and/or improving stability have multiple double bonds, are water soluble, have no net charge or an anionic charge at pH 7, and are not strong hydrogen bonding donors. Not to be bound by theory, the double bond character can allow for weak pi-stacking interactions with protein. In embodiments, at high protein concentrations and in proteins that only develop high viscosity at high concentration, charged excipients are not effective because electrostatic interaction is a longer-range interaction. Solvated protein surfaces are predominantly hydrophilic, making them water soluble. The hydrophobic regions of proteins are generally shielded within the 3-dimensional structure, but the structure is constantly evolving, unfolding, and re-folding (sometimes called “breathing”) and the hydrophobic regions of adjacent proteins can come into contact with each other, leading to aggregation by hydrophobic interactions. The pi-stacking feature of dione and sulfone excipients can mask hydrophobic patches that may be exposed during such “breathing.” Another other important role of the excipient can be to disrupt hydrophobic interactions and hydrogen bonding between proteins in close proximity, which will effectively reduce solution viscosity. Dione and sulfone compounds that fit this description include dimethylsulfone, ethyl methyl sulfone, ethyl methyl sulfonyl acetate, ethyl isopropyl sulfone, sodium cyclamate, bis(methylsulfonyl)methane, methane sulfonamide, methionine sulfone, 1,2-cyclopentanedione, 1,3-cyclopentanedione, 1,4-cyclopentanedione, and butane-2,3-dione.

h. Excipient Compound Category 8: Zwitterionic Excipients

Solutions of therapeutic or non-therapeutic proteins can be formulated with certain zwitterionic compounds as excipients to improve stability or reduce viscosity. As used herein, the term “zwitterionic” refers to a compound that has a cationic charged section and an anionic charged section. In embodiments, the zwitterionic excipient compounds are amine oxides. In embodiments, the opposing charges are separated from each other by 2-8 chemical bonds. In embodiments, the zwitterionic excipient compounds can be small molecules, such as those with a molecular weight of about 50 to about 500 g/mol, or can be medium molecular weight molecules, such as those with a molecular weight of about 500 to about 2000 g/mol, or can be high molecular weight molecules, such as polymers having a molecular weight of about 2000 to about 100,000 g/mol.

Examples of the zwitterionic excipient compounds include (3-carboxypropyl) trimethylammonium chloride, 1-aminocyclohexane carboxylic acid, homocycloleucine, 1-methyl-4-imidazoleacetic acid, 3-(1-pyridinio)-1-propanesulfonate, 4-aminobenzoic acid, alendronate, aminoethyl sulfonic acid, aminohippuric acid, aspartame, aminotris (methylenephosphonic acid) (ATMP), calcobutrol, calteridol, cocamidopropyl betaine, cocamidopropyl hydroxysultaine, creatine, cytidine monophosphate, diaminopimelic acid, diethylenetriaminepentaacetic acid, dimethyl phenylalanine, methylglycine, sarcosine, dimethylglycine, zwitterionic dipeptides (e.g., Arg-Glu, Lys-Glu, His-Glu, Arg-Asp, Lys-Asp, His-Asp, Glu-Arg, Glu-Lys, Glu-His, Asp-Arg, Asp-Lys, Asp-His), diethylenetriamine penta(methylene phosphonic acid) (DTPMP), dipalmitoyl phosphatidylcholine, ectoine, ethylenediamine tetra(methylenephosphonic acid) (EDTMP), folate benzoate mixture, folate niacinamide mixture, gelatin, hydroxyproline, iminodiacetic acid, isoguvacine, lecithin, myristamine oxide, nicotinamide adenine dinucleotide (NAD), N-methyl aspartic acid, N-methylproline, N-trimethyl lysine, ornithine, oxolinic acid, risendronate, allyl cysteine, S-allyl-L-cysteine, somapacitan, taurine, theanine, trigonelline, vigabatrin, ectoine, 4-(2-hydroxyethyl)-1-piperazineethanesulfonate, o-octylphosphoryl choline, nicotinamide mononucleotide, triglycine, tetraglycine, β-guanidinopropionic acid, 5-aminolevulinic acid hydrochloride, picolinic acid, lidofenin, phosphocholine, 1-(5-Carboxypentyl)-4-methylpyridin-1-ium bromide, L-anserine nitrate, L-glutathione reduced, N-ethyl-L-glutamine, N-methyl proline, (Z)-1-[N-(2-aminoethyl)-N-(2-ammonioethyl) amino]diazen-1-ium-1,2-diolate (DETA-NONOate), (Z)-1-[N-(3-aminopropyl)-N-(3-ammoniopropyl)amino]diazen-1-ium-1,2-diolate (DPTA-NONate), and zoledronic acid.

Not to be bound by theory, the zwitterionic excipient compounds can exert viscosity reducing or stabilizing effects by interacting with the protein, for example by charge interactions, hydrophobic interactions, and steric interactions, causing the proteins to be more resistant to aggregation, or by affecting the bulk properties of the water in the protein formulation, such as an electrolyte contribution, a surface tension reduction, a change in the amount of unbound water available, or a change in dielectric constant.

i. Excipient Compound Category 9: Crowding Agents with Hydrogen Bonding Elements

Solutions of therapeutic or non-therapeutic proteins can be formulated with crowding agents with hydrogen bonding elements as excipients to improve stability or reduce viscosity. As used herein, the term “crowding agent” refers to a formulation additive that reduces the amount of water available for dissolving a protein in solution, increasing the effective protein concentration. In embodiments, crowding agents can decrease protein particle size or reduce the amount of protein unfolding in solution. In embodiments, the crowding agents can act as solvent modifiers that cause structuring of the water by hydrogen bonding and hydration effects. In embodiments, the crowding agents can reduce the amount of intermolecular interactions between proteins in solution. In embodiments, the crowding agents have a structure containing at least one hydrogen bond donor element such as hydrogen attached to an oxygen, sulfur, or nitrogen atom. In embodiments, the crowding agents have a structure containing at least one weakly acidic hydrogen bond donor element having a pKa of about 6 to about 11. In embodiments, the crowding agents have a structure containing between about 2 and about 50 hydrogen bond donor elements. In embodiments, the crowding agents have a structure containing at least one hydrogen bond acceptor element such as a Lewis base. In embodiments, the crowding agents have a structure containing between about 2 and about 50 hydrogen bond acceptor elements. In embodiments, the crowding agents have a molecular weight between about 50 and 500 g/mol. In embodiments, the crowding agents have a molecular weight between about 100 and 350 g/mol. In other embodiments, the crowding agents can have a molecular weight above 500 g/mol, such as raffinose, inulin, pullulan, or sinistrins.

Examples of the crowding agent excipients with hydrogen bonding elements include 1,3-Dimethyl-3,4,5,6-tetrahydro-2(1H)-pyrimidione, 15-crown-5, 18-crown-6, 2-butanol, 2-butanone, 2-phenoxyethanol, acetaminophen, allantoin, arabinose, arabitol, benzyl acetonacetate, benzyl alcohol, chlorobutanol, cholestanoltetraacetyl-b-glucoside, cinnamaldehyde, cyclohexanone, deoxyribose, diethyl carbonate, dimethyl carbonate, dimethyl isosorbide, dimethylacetamide, dimethylformamide, dimethylol ethylene urea, dimethyluracil, epilactose, erythritol, erythrose, ethyl lactate, ethyl maltol, ethylene carbonate, formamide, fucose, galactose, genistein, gentisic acid ethanolamide, gluconolactone, glyceraldehyde, glycerol, glycerol carbonate, glycerol formal, glycerol urethane, glycyrrhizic acid, gossypin, harpagoside, hederacoside C, icodextrin, iditol, imidazolidone, inositol, inulins, isomaltitol, kojic acid, lactitol, lactobionic acid, lactose, lactulose, lyxose, madecassoside, maltotriose, mangiferin, mannose, melzitose, methyl lactate, methylpyrrolidone, mogroside V, N-acetylgalactosamine, N-acetylglucosamine, N-acetylneuraminic acid, N-methyl acetamide, N-methyl formamide, N-methyl propionamide, pentaerythritol, pinoresinol diglucoside, piracetam, propyl gallate, propylene carbonate, psicose, pullulan, pyrogallol, quinic acid, raffinose, rebaudioside A, rhamnose, ribitol, ribose, ribulose, saccharin, sedoheptulose, sinistrins, solketal, stachyose, sucralose, tagatose, t-butanol, tetraglycol, triacetin, N-acetyl-d-mannosamine, nystose, kestose, turanose, acarbose, D-saccharic acid 1,4-lactone, thiodigalactoside, fucoidan, hydroxysafflor yellow A, shikimic acid, diosmin, pravastatin sodium salt, D-altrose, L-gulonic gamma-lactone, neomycin, rubusoside dihydroartemisinin, phloroglucinol, naringin, baicalein, hesperidin, apigenin, pyrogallol, morin, salsalate, kaempferol, myricetin, 3′,4′,7-trihydroxyisoflavone, (±)-taxifolin, silybin, perseitol diformal, 4-hydroxyphenylpyruvic acid, sulfacetamide, isopropyl β-D-1-thiogalactopyranoside, ethyl 2,5-dihydroxybenzoate, spectinomycin, resveratrol, quercetin, kanamycin sulfate, 1-(2-Pyrimidyl)piperazine, 2-(2-pyridyl)ethylamine, 2-imidazolidone, DL-1,2-isopropylideneglycerol, metformin, m-xylylenediamine, demeclocycline, tripropylene glycol, tubeimoside I, verbenaloside, xylitol, and xylose.

### 6. Protein/Excipient Solutions: Properties and Processes

In certain embodiments, solutions of therapeutic or non-therapeutic proteins formulated with the above-identified excipient compounds or combinations thereof (hereinafter, “excipient additives”), such as hindered amines, anionic aromatics, functionalized amino acids, oligopeptides, short-chain organic acids, low molecular weight aliphatic polyacids, diones and sulfones, zwitterionic excipients, and crowding agents with hydrogen bonding elements, result in improved protein-protein interaction characteristics as measured by the protein diffusion interaction parameter, kD, or the second virial coefficient, B₂₂. As used herein, an “improvement” in one or more protein-protein interaction parameters achieved by test formulations using the above-identified excipient compounds or combinations thereof can refer to a decrease in attractive protein-protein interactions when a test formulation is compared under comparable conditions with a comparable formulation that does not contain the excipient compounds or excipient additives. Such improvements can be identified by measuring certain parameters that apply to the overall process or an aspect thereof, where a parameter is any metric pertaining to the process where an alteration can be can be quantified and compared to a previous state or to a control. A parameter can pertain to the process itself, such as its efficiency, cost, yield, or rate. Improving the stability of protein containing formulations during processing can have the advantages of improved yield, increased biological activity, and decreased presence of particulates in a formulation.

A parameter can also be a proxy parameter that pertains to a feature or an aspect of the larger process. As an example, parameters such as the kD or B₂₂ parameters can be termed proxy parameters. Measurements of kD and B₂₂ can be made using standard techniques in the industry and can be an indicator of process-related parameters such as improved solution properties or stability of the protein in solution. Not to be bound by theory, it is understood that a highly negative kD value can indicate that the protein has strong attractive interactions, and this can lead to aggregation, instability, and rheology problems. When formulated in the presence of certain of the above-identified excipient compounds or combinations thereof, the same protein can have an improved proxy parameter of a less negative kD value, or a kD value near or above zero, with this improved proxy parameter being associated with an improvement in a process-related parameter.

In embodiments, certain of the above-described excipient compounds or combinations thereof, such as hindered amines, anionic aromatics, functionalized amino acids, oligopeptides, short-chain organic acids, low molecular weight aliphatic polyacids, diones and sulfones, zwitterionic excipients, and crowding agents with hydrogen bonding elements are used to improve a protein-related process, such as the manufacture, processing, sterile filling, purification, and analysis of protein-containing solutions, using processing methods such as filtration, syringing, transferring, pumping, mixing, heating or cooling by heat transfer, gas transfer, centrifugation, chromatography, membrane separation, centrifugal concentration, tangential flow filtration, radial flow filtration, axial flow filtration, lyophilization, and gel electrophoresis. In these and related protein-related processes, the protein of interest is dissolved in a solution that conveys it through the processing apparatus. Such solutions, referred to herein as “carrier solutions,” can include cell culture media (containing, for example, secreted proteins of interest), lysate solutions following the lysis of host cells (where the protein of interest resides in the lysate), elution solutions (which contain the protein of interest following chromatographic separations), electrophoresis solutions, transport solutions for carrying the protein of interest through conduits in a processing apparatus, and the like. A carrier solution containing the protein of interest may also be termed a protein-containing solution or a protein solution. As described in more detail below, one or more above-identified excipient compounds or combinations thereof can be added to the protein-containing solution to improve various aspects of processing. As used herein, the terms “improve,” “improvements,” and the like refer to an advantageous change in a parameter of interest in a carrier solution when that parameter is compared to the same parameter as measured in a control solution. As used herein, a “control solution” means a solution that lacks the viscosity-reducing excipient but otherwise substantially similar to the carrier solution. As used herein, a “control process,” for example a control filtration process, a control chromatographic process, and the like, is a protein-related process that is substantially similar to the protein-related process of interest and is performed with a control solution instead of a carrier solution.

For example, in processes where a protein-containing solution is pumped through conduits (e.g., flow chambers, piping or tubing), it is advantageous to add above-identified excipient compounds or combinations thereof in order to reduce viscosity. Adding a viscosity-lowering excipient to the protein solution, as described above, before or during the pumping process can substantially reduce the force and the power required to pump the solution. It is understood that fluids generally exhibit a resistance to flow, i.e., a viscosity, and that a force must be applied to the fluid to overcome this viscosity in order to induce and propagate flow. The power, P, required for pumping scales with the head, H, and capacity, Q, as shown in the following equation:

P˜HQ (Eq. 1)

Viscous fluids tend to increase the power requirements for pumps, to lower pump efficiency, to decrease pump head and capacity, and to increase frictional resistance in piping. Adding the viscosity-lowering excipients described above to a protein solution prior to or during pumping can substantially lower processing costs by decreasing either the head (H, Eq. 1) or the capacity (Q, Eq. 1) or both. The benefits of reduced viscosity can be manifested, for example, by improved throughput, increased yield, or decreased processing time. Moreover, frictional losses from the transmission of a fluid through a conduit can account for a significant fraction of the costs associated with conveying such fluids. Adding a viscosity-lowering excipient as described above to a protein solution prior to or during pumping can substantially lower processing costs by decreasing the friction accompanying the pumping process. Measurement of processing costs represents a processing parameter that can be improved by using a viscosity-reducing excipient.

These processes and processing methods for protein solutions can have improved efficiency due to the lower viscosity, improved solubility, or improved stability of the proteins in the solution during manufacture, processing, purification, and analysis steps. Measurement of processing efficiency or measurement of proxy parameters such as viscosity, solubility or stability of the proteins in solution represent processing parameters that can be improved by using a viscosity-reducing excipient. Several different factors are understood to adversely affect protein viscosity, solubility, and stability during processing. For example, protein-containing solutions are subject to a variety of physical stressors during manufacturing and purification, including significant shear stresses induced by manipulating protein solutions through typical processing operations, including, but not limited to, pumping, mixing, centrifugation, and filtration. In addition, during these processing steps, air bubbles can become entrained within the fluid to which proteins can adsorb. Such interfacial tension forces, coupled with typical shear stresses encountered during processing, can cause adsorbed protein molecules to unfold and aggregate. Additionally, significant protein unfolding can occur during pump cavitation events and during exposure to solid surfaces during manufacturing, such as ultrafiltration and diafiltration membranes. Such events can impair protein folding and product quality.

For Newtonian fluids, the stress, τ, imposed by a given process scales with the shear rate, \dot{γ}, and viscosity of the fluid, η, as shown in the following equation:

τ=\dot{γ}η (Eq. 2)

By formulating a protein solution with one or more of the above-described excipient compounds or combinations thereof, solution viscosity can be decreased, thus decreasing the shear stress encountered by the protein solution. The decreased shear stress can improve the stability of the formulation being processed, as manifested, for example, by a better or more desirable measurement of a processing parameter. Such improved processing parameters can include metrics such as reduced levels of protein aggregates, particles, or subvisible particles (manifested macroscopically as turbidity), reduced product losses, or improved overall yield. As another example of an improved processing parameter, reducing viscosity of a protein-containing solution can decrease the processing time for the solution. The processing time for a given unit operation generally scales inversely with the shear rate. Therefore, for a given characteristic stress, a decrease in protein solution viscosity by the addition of the above-described excipient compounds or combinations thereof is associated with an increase in shear rate (\dot{γ}, see Eq. 2), and therefore a decrease in the processing time. Moreover, adding certain of the above-identified excipient compounds or combinations thereof can improve the stability of the protein solution during different stages of processing.

During processing, it is understood that a protein in a solution may be a desired protein active ingredient, for example a therapeutic or non-therapeutic protein. Facilitating the processing of such a protein active ingredient using the excipients described herein can increase the yield or the rate of production of the protein active ingredient, or improve the efficiency of the particular process, or decrease the energy use, or the like, any of which outcomes represent processing parameters that have been improved by the use of the viscosity-reducing excipient. It is also understood that protein contaminants can be formed during certain processing technologies, for example during the fermentation and purification steps of bioprocessing. Removing the contaminants more quickly, more thoroughly, or more efficiently can also improve the processing of the desired protein, i.e., the protein active ingredient; these outcomes represent processing parameters that have been improved by the use of the viscosity-reducing excipient compound or additive. As described herein, certain excipients as described herein, by lowering solution viscosity, improving protein stability, and/or increasing protein solubility, can improve the transport of desired protein active ingredients, and can improve the removal of undesirable protein contaminants; both effects, which represent processing parameters that have been improved by the use of the viscosity-reducing excipient or additive, show that these excipients or additives improve the overall process of protein manufacture. A reduction of misfolded protein, particulates, denatured protein, or other artifacts of a destabilized protein in solution can be achieved by use of a stabilizing excipient during processing steps.

Specific platform unit operation for therapeutic protein production and purification offer further examples of the advantageous uses of above-identified excipient compounds or combinations thereof, and further examples of these excipients' or additives' improving processing parameters. As one example, the addition of one or more of the above-identified excipient compounds or combinations thereof can provide substantial improvements in lyophilization processes, resulting in greater efficiency, decrease in operation costs, and/or greater yield of lyophilized protein. Excipients yielding such effects in lyophilization processes can be termed “lyo-improving excipients.” In embodiments, excipients described above as lyo-enhancing excipients can function as lyo-improving excipients as well. Benefits to lyo processes provided by lyo-improving excipients can include improvements such as shorter drying time, faster reconstitution speed, improved process efficiency, energy savings, ability to lyophilize at higher concentration than would be possible without the excipients, improved cake quality, and reduced particle formation.

As another example, introducing one or more of above-identified excipient compounds or combinations thereof into these production and purification processes, as described below, can provide substantial improvements in molecule stability and recovery, and a decrease in operation costs.

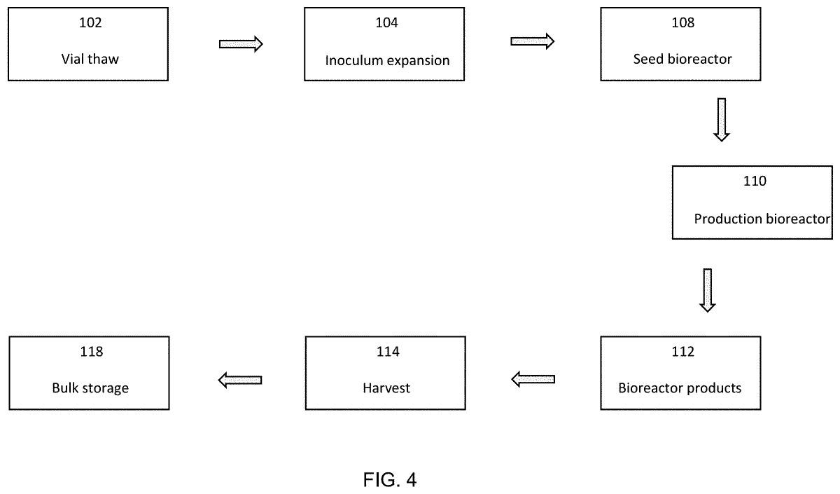

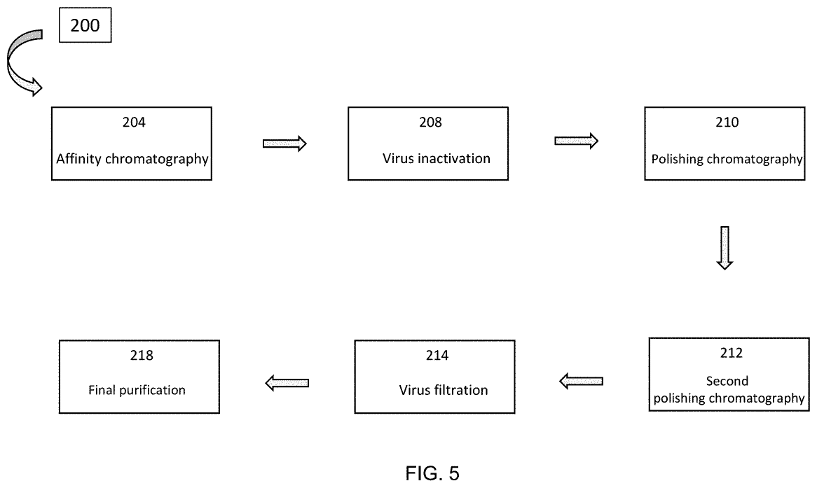

It is understood in the art that the widely-practiced technology for producing and purifying therapeutic proteins like monoclonal antibodies generally consists of a fermentation process followed by a series of steps for purification processing. Fermentation, or upstream processing (USP), comprises those steps by which therapeutic proteins are grown in bioreactors, typically using bacterial or mammalian cell lines. USP may, in embodiments, include steps such as those shown in FIG. 4. Purification, or downstream processing (DSP) may, in embodiments, include steps such as those shown in FIG. 5.

As shown in FIG. 4, USP may commence with the step 102 of thawing of vials from a master cell bank (MCB). The MCB can be expanded as shown in step 104, to form a working cell bank (not shown) and/or to produce the working stock for further production. Cell culture takes place in a series of seed and production bioreactors, as shown in steps 108 and 110, to yield those bioreactor products 112 from which the desired therapeutic protein can be harvested, as shown in step 114. Following harvest 114, the products can be submitted to further purification (i.e., DSP, as described below in more detail and as depicted in FIG. 5), or these products may be stored in bulk, typically by freezing and storing at a temperature of approximately −80° C.

In embodiments, protein production by cell culture techniques can be improved by the use of the above-identified excipients, as manifested by improvements in process-related parameters. In embodiments, the desired excipient can be added during USP in an amount effective to reduce the viscosity of the cell culture medium by at least 20%. In other embodiments, the desired excipient can be added during USP in an amount effective to reduce the viscosity of the cell culture medium by at least 30%. In embodiments, the desired excipient can be added to the cell culture medium in an amount of about 1 mM to about 400 mM. In embodiments, the desired excipient can be added to the cell culture medium in an amount of about 20 mM to about 200 mM. In embodiments, the desired excipient can be added to the cell culture medium in an amount of about 25 mM to about 100 mM. The desired excipient or combination of excipients can be added directly to the cell culture medium, or it can be added as a component of a more complex supplemental medium, for example a nutrient-containing solution or “feed solution” that is formulated separately and added to the cell culture medium. In embodiments, a second excipient, for example, a viscosity-reducing compound, can be added to the carrier solution, either directly or via a supplemental medium, wherein the second viscosity-reducing compound adds an additional improvement to a particular parameter of interest.

As described below, there are many process-related parameters during USP that can be improved by use of one or more of the above-identified excipient compounds or combinations thereof. For example, in embodiments, use of a viscosity-reducing excipient can improve parameters such as the rate and/or degree of cell growth during steps such as inoculum expansion 104, and cell culture 108 and 110, and/or can improve proxy parameters that are correlated with the improvement in various process parameters. For example, adding certain of the above-identified excipients to the USP process at a step such as the production bioreactor step 110, can decrease the viscosity of the cell culture medium, which can subsequently improve heat transfer efficiency and gas transfer efficiency. Because the cell culture process requires oxygen infusion to the cells to enable protein expression, and the diffusion of oxygen into the cells can therefore be a rate-limiting step, improving the rate of oxygen uptake by improving gas transfer efficiency through decreasing solution viscosity can improve the rate or amount of protein expression and/or its efficiency. In this context, parameters such as the rate of oxygen uptake and the rate of gas transfer efficiency can be deemed proxy parameters, whose improvement is correlated with an improvement in the process parameter of improved protein expression or improved processing efficiency. As another example, the availability of viscosity-reducing excipients can improve processing, for example, during the inoculum expansion step 104 and during the cell culture steps 108 and 110, by improving a proxy parameter such as the solubility of protein growth factors that are required for protein expression; with improved growth factor solubility, these substances can become more available to the cells, thereby facilitating cell growth.

In embodiments, process parameters such as the amount of protein recovery or the rate of protein recovery during USP can be improved by reducing viscosity during USP by several mechanisms. For example, the harvest of therapeutic protein at the end of the lysis step during harvest 114 from the completed cell culture can be more efficient or can be otherwise improved with the use of the above-identified excipients. Not to be bound by theory, by reducing viscosity of the expressed protein, these viscosity-reducing excipients can increase the efficiency of diffusion of therapeutic protein away from other lysate components. In addition, the separation of membranes and other cell debris from the protein-containing supernate can be accomplished with a faster separation rate or a higher degree of supernate purity, with the use of the viscosity-reducing excipients, thereby improving the process parameter of USP efficiency. Furthermore, the protein separation steps that use centrifugation or filtration steps can be accomplished faster with the use of the viscosity-reducing excipients, since the excipients reduce the viscosity of the medium. Since the excipients can also improve stability of therapeutic protein solutions, the upstream and downstream processing of proteins can benefit from the use of these excipients. In embodiments, the excipients can improve the stress tolerance of the proteins during processing, and this can reduce the amount of aggregation or denaturation of the protein during the processing steps.

In embodiments, as an additional benefit, use in cell culture of the above-described excipient compounds or combinations thereof, for example viscosity-reducing excipients, can increase a process parameter such as protein yield during USP because protein misfolding and aggregation are reduced. It is understood that, as the cell culture is optimized to produce a maximum yield of recombinant protein, the resulting protein is expressed in a highly concentrated manner, which can result in misfolding; adding the above-identified excipient compounds or combinations thereof, for example a viscosity-reducing excipient, can reduce the attractive protein-protein interactions that lead to misfolding and aggregation, thereby increasing the amount of intact recombinant protein that is available for harvest 114.

Downstream processing (DSP), depicted in FIG. 5 in an illustrative embodiment, involves a sequence of steps that results in the recovery and purification of therapeutic proteins, for example monoclonal antibodies, biopharmaceuticals, vaccines, and other biologics. At the end of USP, the therapeutic protein of interest can be dissolved in the cell culture medium, having been secreted from the host cells. The therapeutic protein can also be dissolved in a fluid medium following the lysis of the host cells at the end of the USP sequences. DSP is undertaken to retrieve the protein of interest from the solution in which it is dissolved (e.g., the culture medium or host cell lysate medium), and to purify it. During DSP, (i) various contaminants (such as insoluble cell debris and particulates) are removed from the media, (ii) the protein product is isolated through techniques such as extraction, precipitation, adsorption or ultrafiltration, (iii) the protein product is purified through techniques such as affinity chromatography, precipitation, or crystallization, and (iv) the product is further polished, and viruses are removed.

As shown in FIG. 5, a feedstock from cell culture harvest 200 (also as described in FIG. 4) is initially subjected to affinity chromatography 204, typically involving Protein-A chromatography or other analogous chromatographic steps. The virus inactivation step 208 typically entails subjecting the feedstock to a low pH hold. One or more polishing chromatography steps 210 and 212 are performed to remove impurities, such as host cell proteins (HCP), DNA, charge variants, and aggregates. Cation exchange (CEX) chromatography is commonly used as an initial polishing chromatography step 210, but it may be accompanied by a second chromatography step 212 that either precedes or follows it. The second chromatography step 212 further removes host-cell-related impurities (e.g., HCP or DNA), or product related impurities such as aggregates. Anion exchange (AEX) chromatography and hydrophobic interaction chromatography (HIC) can be employed as second chromatography steps 212. Virus filtration 214 is performed to effect virus removal. Final purification steps 218 can include ultrafiltration and diafiltration, and preparation for formulation.

As generally described above, purification processes or DSP following the fermentation process can include (1) cell culture harvest, (2) chromatography (e.g., Protein-A chromatography and chromatographic polishing steps, including ion exchange and hydrophobic interaction chromatography), (3) viral inactivation, and (4) filtration (e.g., viral filtration, sterile filtration, dialysis, and ultrafiltration and diafiltration steps to concentrate the protein and exchange the protein into the formulation buffer). Examples are provided below to illustrate the advantages from using the above-identified excipient compounds or combinations thereof, for example a viscosity-reducing excipient, to improve process parameters associated with these purification processes. It is understood that the above-identified excipient compounds or combinations thereof, for example a viscosity-reducing excipient, can be introduced at any phase of DSP by adding it to a carrier solution or in any other way engineering the contact of the protein of interest with the excipient, whether in soluble or stabilized form. In embodiments, a second excipient, for example, a viscosity-reducing compound, can be added to the carrier solution during DSP, wherein the second compound adds an additional improvement to a particular parameter of interest.

(1) Cell culture harvest: Cell culture harvest generally involves centrifugation and depth filtration operations in which cellular debris is physically removed from protein-containing solutions. The centrifugation step can provide a more complete separation of soluble protein from cell debris with the benefit of a viscosity-reducing excipient. Whether done by batch or continuous processing, the centrifuge separation requires the dense phase to consolidate as much as possible to maximize recovery of the target protein. In embodiments, addition of the above-identified excipients or combinations thereof can increase the process parameter of protein yield, for example, by increasing the yield of protein-containing centrate that flows away from the dense phase of the centrifuge separation process. The depth filtration step is a viscosity-limited step, and thus can be made more efficient by using an excipient that reduces solution viscosity. These processes can also introduce air bubbles into the protein solution, which can couple with shear-induced stresses to destabilize the therapeutic protein molecules being purified. Adding a viscosity-reducing excipient to the protein-containing solution, before and/or during cell culture harvest, as described above, can protect the protein from these stresses, thereby reducing the likelihood of protein aggregation and improving the process parameter of quantified product recovery.

(2) Chromatography. After cell culture harvest by centrifugation or filtration, chromatography is typically used to separate the therapeutic protein from the fermentation broth. Protein A chromatography is used when the therapeutic protein is an antibody: Protein A is selective towards IgG antibodies, which it will bind dynamically at a high flow rate and capacity. Cation exchange (CEX) chromatography can be used as a cost-effective alternative to Protein A chromatography. If CEX is used, the pH of the feed must be adjusted and its conductivity decreased prior to loading onto the column to optimize the dynamic binding capacity. Mimetic resins can also be used as an alternative to Protein A chromatography. These resins provide ligands to bind immunoglobulins, for example Ig-binding proteins like protein G or protein L, synthetic ligands, or protein A-like porous polymers.

Other chromatography processes can be employed during DSP. Ion exchange chromatography (IEC) can be used to remove impurities introduced during previous processes, for example, leached Protein A, endotoxins or viruses from the cell line, remaining host cell proteins or DNA, or media components. IEC, whether CEX or anion exchange chromatography, can be applied directly after Protein A chromatography. Hydrophobic interaction chromatography (HIC) can complement IEC, generally used as a polishing step to remove aggregates. In embodiments, the use of the above-identified excipients can increase the solubility of and decrease the viscosity of host cell proteins during chromatography column loading steps. In embodiments, the use of the above-identified excipients can increase the solubility of and decrease the viscosity of the therapeutic protein during chromatography column loading steps and elution steps.

Chromatographic processes during protein purification impose harsh conditions on the protein formulation, such as (a) low pH conditions during elution from Protein-A chromatography columns, (b) elevated local protein concentration (often on the order of 300-400 mg/mL) within the pore-space of chromatographic resin, (c) elevated salt concentrations during ion exchange chromatography, and (d) elevated concentrations of salting-out agents during elution from HIC columns. Adding a viscosity-reducing excipient to the protein-containing solution, before and/or during chromatography, as described above, can facilitate the transit of the proteins through the chromatography column so that they are less exposed to the potentially damaging conditions imposed by chromatographic processing steps. In addition, the elevated local protein concentration within the column pore-space can result in a highly viscous material within this space, which places significant back pressure on the column. To alleviate this back pressure, media with relatively large pores are typically used. However, the resolving power of large-pore media is lower than small-pore counterparts. The incorporation of viscosity-modifying excipients as described above can enable the use of smaller pores in the chromatographic media. In embodiments, the elution steps from Protein-A chromatography expose the therapeutic protein to a low pH condition that can reduce solubility and increase aggregation of the target protein; addition of the excipients can increase the solubility of the target protein such that recovery yield from the Protein-A chromatography step is improved. In other embodiments, use of the excipient can enable elution of the target protein from Protein-A resin at a higher pH, and this can reduce chemical stresses on the target protein, resulting in improving a process parameter of protein yield by reducing the amount of protein degradation during processing.

(3) Viral inactivation: Viral inactivation processes typically involve holding the protein solution at a low pH, e.g., pH lower than 4, for an extended period of time. This environment, though, can destabilize therapeutic proteins. Formulating the protein in the presence of a viscosity-reducing excipient, for example, by adding a viscosity-reducing excipient before and/or during a viral inactivation process, can improve process parameters such as the stability or solubility of the protein, or its net yield.

(4) Filtration: Filtration processes include viral filtration processes (nanofiltration) to remove virus particles, and ultrafiltration/diafiltration processes to concentrate protein solutions and to exchange buffer systems.

(a) Viral filtration purifies the protein solution by removing virus particles, which can be on the order of twice the size of a recombinant human monoclonal antibodies. Thus, the filtration membrane for viral filtration can require nano-sized pores. As a result of the small pore size through which the proteins must pass, this filtration step can introduce stress to the protein, and is accompanied by significant levels of membrane fouling from protein aggregate particles. The addition of a viscosity-reducing excipient, for example, before and/or during filtration, as described above, can reduce a measurable parameter such as back pressure in the filtration system by increasing collective diffusivity, and can decrease the tendency for membrane fouling by mitigating the protein-protein interactions that give rise to it. The end result is improvement in those parameters indicting improved performance of the viral filtration unit during protein purification.

(b) Ultrafiltration and diafiltration (UF/DF) processes concentrate protein solutions and exchange buffer systems by passing the protein-containing solution through a filter membrane with a characteristic molecular weight cutoff that is smaller than the protein of interest. In this step, the protein solution faces high shear stresses within the filter units, elevated protein concentrations, and adsorption of the protein to the hydrophobic membranes typically used during UF/DF processes, all of which can increase protein aggregation. The addition of a viscosity-reducing excipient, for example, before and/or during a UF/DF process, as described above, can reduce back pressure in the filtration system by increasing collective diffusivity (measured, for example, by an increase in kD). This not only reduces shear stress across the membrane, but also promotes back-diffusion away from the filter membrane, thus lowering the effective protein concentration at the membrane interface and increasing the permeate flux. As a result, the use of viscosity-reducing excipients can improve parameters associated with higher throughput during these filtration processes, with reduced product losses and increased net yield. Additionally, passing viscous fluids through ultra- and diafilters can produce a large pressure drop across the filter device, making the separation inefficient. Formulating the protein solution in the presence of viscosity-reducing excipients as described above can substantially reduce the pressure drop across the filter device, thereby improving the process parameters of operation costs and processing time by decreasing them both.

After the upstream protein processing or downstream purification have been completed with the added excipient, the excipient can remain as a part of the drug substance mixture or it can be separated from the protein active ingredient. Typical small molecule separation methods can be used to separate the excipient from the protein active ingredient, such as buffer exchange, ion exchange, ultrafiltration, and dialysis. In addition to the beneficial effects on the protein purification processes as outlined above, the use of the above-identified excipients can protect and preserve equipment used in protein manufacture, processing, and purification. For example, equipment-related processes such as the cleanup, sterilization, and maintenance of protein processing equipment can be facilitated by the use of the above-identified excipients due to decreased fouling, decreased denaturing, lower viscosity, and improved solubility of the protein, and parameters associated with the improvement of these processes are similarly improved.

While the use of an excipient compound to improve upstream and/or downstream processing has been described extensively herein, it is understood that a combination of excipients can be added together in order to achieve a desired effect, such as an improvement in a parameter of interest. The term “excipient additive” can refer to either a single excipient compound that leads to the desired effect or improved parameter, or to a combination of excipient compounds where the combination is responsible for the desired effect or the improved parameter.

### EXAMPLES

Materials:
Bovine gamma globulin (BGG), >99% purity, Catalog #G5009, Sigma Aldrich
Histidine, Sigma Aldrich
Other materials described in the examples below were from Sigma Aldrich unless otherwise specified.

### Example 1: Preparation of Formulations Containing Excipient Compounds and Test Protein

Formulations were prepared using an excipient compound and a test protein, where the test protein was intended to simulate either a therapeutic protein that would be used in a therapeutic formulation, or a non-therapeutic protein that would be used in a non-therapeutic formulation. Such formulations were prepared in 50 mM histidine hydrochloride with different excipient compounds for viscosity measurement in the following way. Histidine hydrochloride was first prepared by dissolving 1.94 g histidine in distilled water and adjusting the pH to about 6.0 with 1 M hydrochloric acid (Sigma-Aldrich, St. Louis, Mo.) and then diluting to a final volume of 250 mL with distilled water in a volumetric flask. Excipient compounds were then dissolved in 50 mM histidine HCl. Lists of excipients are provided below in Examples 4, 5, 6, and 7. In some cases excipient compounds were adjusted to pH 6 prior to dissolving in 50 mM histidine HCl. In this case the excipient compounds were first dissolved in deionized water at about 5 wt % and the pH was adjusted to about 6.0 with either hydrochloric acid or sodium hydroxide. The prepared salt solution was then placed in a convection laboratory oven at about 65° C. to evaporate the water and isolate the solid excipient. Once excipient solutions in 50 mM histidine HCl had been prepared, the test protein bovine gamma globulin (BGG) was dissolved at a ratio of about 0.336 g BGG per 1 mL excipient solution. This resulted in a final protein concentration of about 280 mg/mL. Solutions of BGG in 50 mM histidine HCl with excipient were formulated in 20 mL vials and allowed to shake at 100 rpm on an orbital shaker table overnight. BGG solutions were then transferred to 2 mL microcentrifuge tubes and centrifuged for ten minutes at 2300 rpm in an IEC MicroMax microcentrifuge to remove entrained air prior to viscosity measurement.

### Example 2: Viscosity Measurement

Viscosity measurements of formulations prepared as described in Example 1 were made with a DV-IIT LV cone and plate viscometer (Brookfield Engineering, Middleboro, Mass.). The viscometer was equipped with a CP-40 cone and was operated at 3 rpm and 25° C. The formulation was loaded into the viscometer at a volume of 0.5 mL and allowed to incubate at the given shear rate and temperature for 3 minutes, followed by a measurement collection period of twenty seconds. This was then followed by 2 additional steps consisting of 1 minute of shear incubation and subsequent twenty-second measurement collection period. The three data points collected were then averaged and recorded as the viscosity for the sample.

### Example 3: Protein Concentration Measurement

The concentration of the protein in the experimental solutions was determined by measuring the optical absorbance of the protein solution at a wavelength of 280 nm in a UV/VIS Spectrometer (Perkin Elmer Lambda 35). First the instrument was calibrated to zero absorbance with a 50 mM histidine buffer at pH 6. Next the protein solutions were diluted by a factor of 300 with the same histidine buffer and the absorbance at 280 nm recorded. The final concentration of the protein in the solution was calculated by using the extinction coefficient value of 1.264 mL/(mg×cm).

### Example 4: Formulations with Hindered Amine Excipient Compounds

Formulations containing 280 mg/mL BGG were prepared as described in Example 1, with some samples containing added excipient compounds. In these tests, the hydrochloride salts of dimethylcyclohexylamine (DMCHA), dicyclohexylmethylamine (DCHMA), dimethylaminopropylamine (DMAPA), triethanolamine (TEA), dimethylethanolamine (DMEA), and niacinamide were tested as examples of the hindered amine excipient compounds. Also, a hydroxybenzoic acid salt of DMCHA and a taurine-dicyandiamide adduct were tested as examples of the hindered amine excipient compounds. The viscosity of each protein solution was measured as described in Example 2, and the results are presented in Table 1 below, showing the benefit of the added excipient compounds in reducing viscosity.

TABLE 1

| | Excipient | | |
|---|---|---|---|
| | Concentration | Viscosity | Viscosity |
| Excipient Added | (mg/mL) | (cP) | Reduction |
| None | 0 | 79 | 0% |
| DMCHA-HCl | 28 | 50 | 37% |
| DMCHA-HCl | 41 | 43 | 46% |
| DMCHA-HCl | 50 | 45 | 43% |
| DMCHA-HCl | 82 | 36 | 54% |
| DMCHA-HCl | 123 | 35 | 56% |
| DMCHA-HCl | 164 | 40 | 49% |
| DMAPA-HCl | 87 | 57 | 28% |
| DMAPA-HCl | 40 | 54 | 32% |
| DCHMA-HCl | 29 | 51 | 35% |
| DCHMA-HCl | 50 | 51 | 35% |
| TEA-HCl | 97 | 51 | 35% |
| TEA-HCl | 38 | 57 | 28% |
| DMEA-HCl | 51 | 51 | 35% |
| DMEA-HCl | 98 | 47 | 41% |
| DMCHA-hydroxybenzoate | 67 | 46 | 42% |
| DMCHA-hydroxybenzoate | 92 | 42 | 47% |
| Product of Example 8 | 26 | 58 | 27% |
| Product of Example 8 | 58 | 50 | 37% |
| Product of Example 8 | 76 | 49 | 38% |
| Product of Example 8 | 103 | 46 | 42% |
| Product of Example 8 | 129 | 47 | 41% |
| Product of Example 8 | 159 | 42 | 47% |
| Product of Example 8 | 163 | 42 | 47% |
| Niacinamide | 48 | 39 | 51% |
| N-Methyl-2-pyrrolidone | 30 | 45 | 43% |
| N-Methyl-2-pyrrolidone | 52 | 52 | 34% |

### Example 5: Formulations with Anionic Aromatic Excipient Compounds

Formulations of 280 mg/mL BGG were prepared as described in Example 1, with some samples containing added excipient compounds. The viscosity of each solution was measured as described in Example 2, and the results are presented in Table 2 below, showing the benefit of the added excipient compounds in reducing viscosity.

TABLE 2

| | Excipient | | |
|---|---|---|---|
| | Concentration | Viscosity | Viscosity |
| Excipient | (mg/mL) | (cP) | Reduction |
| None | 0 | 79 | 0% |
| Sodium aminobenzoate | 43 | 48 | 39% |
| Sodium hydroxybenzoate | 26 | 50 | 37% |
| Sodium sulfanilate | 44 | 49 | 38% |
| Sodium sulfanilate | 96 | 42 | 47% |
| Sodium indole acetate | 52 | 58 | 27% |
| Sodium indole acetate | 27 | 78 | 1% |
| Vanillic acid, sodium salt | 25 | 56 | 29% |
| Vanillic acid, sodium salt | 50 | 50 | 37% |
| Sodium salicylate | 25 | 57 | 28% |
| Sodium salicylate | 50 | 52 | 34% |
| Adenosine monophosphate | 26 | 47 | 41% |
| Adenosine monophosphate | 50 | 66 | 16% |
| Sodium benzoate | 31 | 61 | 23% |
| Sodium benzoate | 56 | 62 | 22% |

### Example 6: Formulations with Oligopeptide Excipient Compounds

Oligopeptides (n=5) were synthesized by NeoflioLab Inc. (Woburn, Mass.) in >9500 purity with the N terminus as a free amine and the C terminus as a free acid. Dipeptides (n=2) were synthesized by LifeTein LLC (Somerset, N.J.) in 950% purity. Formulations of 280 mg/mL BGG were prepared as described in Example 1, with some samples containing the synthetic oligopeptides as added excipient compounds. The viscosity of each solution was measured as described in Example 2, and the results are presented in Table 3 below, showing the benefit of the added excipient compounds in reducing viscosity.

TABLE 3

| | Excipient | | |
|---|---|---|---|
| | Concentration | Viscosity | Viscosity |
| Excipient Added | (mg/mL) | (cP) | Reduction |
| None | 0 | 79 | 0% |
| ArgX5 | 100 | 55 | 30% |
| ArgX5 | 50 | 54 | 32% |
| HisX5 | 100 | 62 | 22% |
| HisX5 | 50 | 51 | 35% |
| HisX5 | 25 | 60 | 24% |
| Trp2Lys3 | 100 | 59 | 25% |
| Trp2Lys3 | 50 | 60 | 24% |
| AspX5 | 100 | 102 | −29% |
| AspX5 | 50 | 82 | −4% |
| Dipeptide LE (Leu-Glu) | 50 | 72 | 9% |
| Dipeptide YE (Tyr-Glu) | 50 | 55 | 30% |
| Dipeptide RP (Arg-Pro) | 50 | 51 | 35% |
| Dipeptide RK (Arg-Lys) | 50 | 53 | 33% |
| Dipeptide RH (Arg-His) | 50 | 52 | 34% |
| Dipeptide RR (Arg-Arg) | 50 | 57 | 28% |
| Dipeptide RE (Arg-Glu) | 50 | 50 | 37% |
| Dipeptide LE (Leu-Glu) | 100 | 87 | −10% |
| Dipeptide YE (Tyr-Glu) | 100 | 68 | 14% |
| Dipeptide RP (Arg-Pro) | 100 | 53 | 33% |
| Dipeptide RK (Arg-Lys) | 100 | 64 | 19% |
| Dipeptide RH (Arg-His) | 100 | 72 | 9% |
| Dipeptide RR (Arg-Arg) | 100 | 62 | 22% |
| Dipeptide RE (Arg-Glu) | 100 | 66 | 16% |

### Example 7: Synthesis of Guanyl Taurine Excipient

Guanyl taurine was prepared following the method described in U.S. Pat. No. 2,230,965. Taurine (Sigma-Aldrich, St. Louis, Mo.) 3.53 parts were mixed with 1.42 parts of dicyandiamide (Sigma-Aldrich, St. Louis, Mo.) and grinded in a mortar and pestle until a homogeneous mixture was obtained. Next the mixture was placed in a flask and heated at 200° C. for 4 hours. The product was used without further purification.

### Example 8: Protein Formulations Containing Excipient Compounds

Formulations were prepared using an excipient compound and a test protein, where the test protein was intended to simulate either a therapeutic protein that would be used in a therapeutic formulation, or a non-therapeutic protein that would be used in a non-therapeutic formulation. Such formulations were prepared in 50 mM aqueous histidine hydrochloride buffer solution with different excipient compounds for viscosity measurement in the following way. Histidine hydrochloride buffer solution was first prepared by dissolving 1.94 g histidine in distilled water and adjusting the pH to about 6.0 with 1 M hydrochloric acid (Sigma-Aldrich, St. Louis, Mo.) and then diluting to a final volume of 250 mL with distilled water in a volumetric flask. Excipient compounds were then dissolved in the 50 mM histidine HCl buffer solution. A list of the excipient compounds is provided in Table 4. In some cases, excipient compounds were dissolved in 50 mM histidine HCl buffer solution and the resulting solution pH was adjusted with small amounts of sodium hydroxide or hydrochloric acid to achieve pH 6 prior to dissolution of the model protein. In some cases, excipient compounds were adjusted to pH 6 prior to dissolving in 50 mM histidine HCl. In this case the excipient compounds were first dissolved in deionized water at about 5 wt % and the pH was adjusted to about 6.0 with either hydrochloric acid or sodium hydroxide. The prepared salt solution was then placed in a convection laboratory oven at about 65° C. to evaporate the water and isolate the solid excipient. Once excipient solutions in 50 mM histidine HCl had been prepared, the test protein, bovine gamma globulin (BGG) was dissolved at a ratio to achieve a final protein concentration of about 280 mg/mL. Solutions of BGG in 50 mM histidine HCl with excipient were formulated in 20 mL vials and allowed to shake at 100 rpm on an orbital shaker table overnight. BGG solutions were then transferred to 2 mL microcentrifuge tubes and centrifuged for ten minutes at 2300 rpm in an IEC MicroMax microcentrifuge to remove entrained air prior to viscosity measurement.

Viscosity measurements of formulations prepared as described above were made with a DV-IIT LV cone and plate viscometer (Brookfield Engineering, Middleboro, Mass.). The viscometer was equipped with a CP-40 cone and was operated at 3 rpm and 25° C. The formulation was loaded into the viscometer at a volume of 0.5 mL and allowed to incubate at the given shear rate and temperature for 3 minutes, followed by a measurement collection period of twenty seconds. This was then followed by 2 additional steps consisting of 1 minute of shear incubation and subsequent twenty-second measurement collection period. The three data points collected were then averaged and recorded as the viscosity for the sample. Viscosities of solutions with excipient were normalized to the viscosity of the model protein solution without excipient. The normalized viscosity is the ratio of the viscosity of the model protein solution with excipient to the viscosity of the model protein solution with no excipient.

TABLE 4

| | Excipient | Normalized | |
|---|---|---|---|
| | Concentration | Viscosity | Viscosity |
| Excipient Added | (mg/mL) | (cP) | Reduction |
| DMCHA-HCl | 120 | 0.44 | 56% |
| Niacinamide | 50 | 0.51 | 49% |
| Isonicotinamide | 50 | 0.48 | 52% |
| Tyramine HCl | 70 | 0.41 | 59% |
| Histamine HCl | 50 | 0.41 | 59% |
| Imidazole HCl | 100 | 0.43 | 57% |
| 2-methyl-2-imidazoline HCl | 60 | 0.43 | 57% |
| 1-butyl-3-methylimidazolium | 100 | 0.48 | 52% |
| chloride | | | |
| Procaine HCl | 50 | 0.53 | 47% |
| 3-aminopyridine | 50 | 0.51 | 49% |
| 2,4,6-trimethylpyridine | 50 | 0.49 | 51% |
| 3-pyridine methanol | 50 | 0.53 | 47% |
| Nicotinamide adenine | 20 | 0.56 | 44% |
| dinucleotide | | | |
| Sodium phenylpyruvate | 55 | 0.57 | 43% |
| 2-Pyrrolidinone | 60 | 0.68 | 32% |
| Morpholine HCl | 50 | 0.60 | 40% |
| Agmatine sulfate | 55 | 0.77 | 23% |
| 1-butyl-3-methylimidazolium | 60 | 0.66 | 34% |
| iodide | | | |
| L-Anserine nitrate | 50 | 0.79 | 21% |
| 1-hexyl-3-methylimidazolium | 65 | 0.89 | 11% |
| chloride | | | |
| N,N-diethyl nicotinamide | 50 | 0.67 | 33% |
| Nicotinic acid, sodium salt | 100 | 0.54 | 46% |
| Biotin | 20 | 0.69 | 31% |

### Example 9: Preparation of Formulations Containing Excipient Combinations and Test Protein

Formulations were prepared using a primary excipient compound, a secondary excipient compound and a test protein, where the test protein was intended to simulate either a therapeutic protein that would be used in a therapeutic formulation, or a non-therapeutic protein that would be used in a non-therapeutic formulation. The primary excipient compounds were selected from compounds having both anionic and aromatic functionality, as listed below in Table 5. The secondary excipient compounds were selected from compounds having either nonionic or cationic charge at pH 6 and either imidazoline or benzene rings, as listed below in Table 5. Formulations of these excipients were prepared in 50 mM histidine hydrochloride buffer solution for viscosity measurement in the following way. Histidine hydrochloride was first prepared by dissolving 1.94 g histidine in distilled water and adjusting the pH to about 6.0 with 1 M hydrochloric acid (Sigma-Aldrich, St. Louis, Mo.) and then diluting to a final volume of 250 mL with distilled water in a volumetric flask. The individual primary or secondary excipient compounds were then dissolved in 50 mM histidine HCl. Combinations of primary and secondary excipients were dissolved in 50 mM histidine HCl and the resulting solution pH adjusted with small amounts of sodium hydroxide or hydrochloric acid to achieve pH 6 prior to dissolution of the model protein. Once excipient solutions had been prepared as described above, the test protein bovine gamma globulin (BGG) was dissolved into each test solution at a ratio to achieve a final protein concentration of about 280 mg/mL. Solutions of BGG in 50 mM histidine HCl with excipient were formulated in 20 mL vials and allowed to shake at 100 rpm on an orbital shaker table overnight. BGG solutions were then transferred to 2 mL microcentrifuge tubes and centrifuged for ten minutes at 2300 rpm in an IEC MicroMax microcentrifuge to remove entrained air prior to viscosity measurement.

Viscosity measurements of formulations prepared as described above were made with a DV-IIT LV cone and plate viscometer (Brookfield Engineering, Middleboro, Mass.). The viscometer was equipped with a CP-40 cone and was operated at 3 rpm and 25° C. The formulation was loaded into the viscometer at a volume of 0.5 mL and allowed to incubate at the given shear rate and temperature for 3 minutes, followed by a measurement collection period of twenty seconds. This was then followed by 2 additional steps consisting of 1 minute of shear incubation and a subsequent twenty-second measurement collection period. The three data points collected were then averaged and recorded as the viscosity for the sample. Viscosities of solutions with excipient were normalized to the viscosity of the model protein solution without excipient and summarized in Table 5 below. The normalized viscosity is the ratio of the viscosity of the model protein solution with excipient to the viscosity of the model protein solution with no excipient. The example shows that a combination of primary and secondary excipients can give a better result than a single excipient.

TABLE 5

| Primary | Concen- | Secondary | Concen- | |
|---|---|---|---|---|
| Excipient | tration | Excipient | tration | Normalized |
| Name | (mg/mL) | Name | (mg/mL) | Viscosity |
| Salicylic Acid | 30 | None | 0 | 0.79 |
| Salicylic Acid | 25 | Imidazole | 4 | 0.59 |
| 4-hydroxybenzoic | 30 | None | 0 | 0.61 |
| acid | | | | |
| 4-hydroxybenzoic | 25 | Imidazole | 5 | 0.57 |
| acid | | | | |
| 4-hydroxybenzene | 31 | None | 0 | 0.59 |
| sulfonic acid | | | | |
| 4-hydroxybenzene | 26 | Imidazole | 5 | 0.70 |
| sulfonic acid | | | | |
| 4-hydroxybenzene | 25 | Caffeine | 5 | 0.69 |
| sulfonic acid | | | | |
| None | 0 | Caffeine | 10 | 0.73 |
| None | 0 | Imidazole | 5 | 0.75 |

### Example 10: Preparation of Formulations Containing Excipient Combinations and Test Protein

Formulations were prepared using a primary excipient compound, a secondary excipient compound and a test protein, where the test protein was intended to simulate a therapeutic protein that would be used in a therapeutic formulation, or a non-therapeutic protein that would be used in a non-therapeutic formulation. The primary excipient compounds were selected from compounds having both anionic and aromatic functionality, as listed below in Table 6. The secondary excipient compounds were selected from compounds having either nonionic or cationic charge at pH 6 and either imidazoline or benzene rings, as listed below in Table 6. Formulations of these excipients were prepared in distilled water for viscosity measurement in the following way. Combinations of primary and secondary excipients were dissolved in distilled water and the resulting solution pH adjusted with small amounts of sodium hydroxide or hydrochloric acid to achieve pH 6 prior to dissolution of the model protein. Once excipient solutions in distilled water had been prepared, the test protein bovine gamma globulin (BGG) was dissolved at a ratio to achieve a final protein concentration of about 280 mg/mL. Solutions of BGG in distilled water with excipient were formulated in 20 mL vials and allowed to shake at 100 rpm on an orbital shaker table overnight. BGG solutions were then transferred to 2 mL microcentrifuge tubes and centrifuged for ten minutes at 2300 rpm in an IEC MicroMax microcentrifuge to remove entrained air prior to viscosity measurement.

Viscosity measurements of formulations prepared as described above were made with a DV-IIT LV cone and plate viscometer (Brookfield Engineering, Middleboro, Mass.). The viscometer was equipped with a CP-40 cone and was operated at 3 rpm and 25° C. The formulation was loaded into the viscometer at a volume of 0.5 mL and allowed to incubate at the given shear rate and temperature for 3 minutes, followed by a measurement collection period of twenty seconds. This was then followed by 2 additional steps consisting of 1 minute of shear incubation and a subsequent twenty-second measurement collection period. The three data points collected were then averaged and recorded as the viscosity for the sample. Viscosities of solutions with excipient were normalized to the viscosity of the model protein solution without excipient and summarized in Table 6 below. The normalized viscosity is the ratio of the viscosity of the model protein solution with excipient to the viscosity of the model protein solution with no excipient. The example shows that a combination of primary and secondary excipients can give a better result than a single excipient.

TABLE 6

| Primary Excipient | | Secondary Excipient | | |
|---|---|---|---|---|
| | Concen- | | Concen- | |
| | tration | | tration | Normalized |
| Name | (mg/mL) | Name | (mg/mL) | Viscosity |
| Salicylic Acid | 20 | None | 0 | 0.96 |
| Salicylic Acid | 20 | Caffeine | 5 | 0.71 |
| Salicylic Acid | 20 | Niacinam | 5 | 0.76 |
| Salicylic Acid | 20 | Imidazol | 5 | 0.73 |

### Example 11: Preparation of Formulations Containing Excipient Compounds and PEG

Materials: All materials were purchased from Sigma-Aldrich, St. Louis, Mo. Formulations were prepared using an excipient compound and PEG, where the PEG was intended to simulate a therapeutic PEGylated protein that would be used in a therapeutic formulation. Such formulations were prepared by mixing equal volumes of a solution of PEG with a solution of the excipient. Both solutions were prepared in a Tris buffer consisting of 10 mM Tris, 135 mM NaCl, 1 mM trans-cinnamic acid at pH of 7.3. The PEG solution was prepared by mixing 3 g of poly(ethylene oxide) average Mw ˜1,000,000 (Aldrich Catalog #372781) with 97 g of the Tris buffer solution. The mixture was stirred overnight for complete dissolution.

An example of the excipient solution preparation is as follows: An approximately 80 mg/mL solution of citric acid in the Tris buffer was prepared by dissolving 0.4 g of citric acid (Aldrich cat. #251275) in 5 mL of the Tris buffer solution and adjusted the pH to 7.3 with minimum amount of 10 M NaOH solution. The PEG excipient solution was prepared by mixing 0.5 mL of the PEG solution with 0.5 mL of the excipient solution and mixed by using a vortex for a few seconds. A control sample was prepared by mixing 0.5 mL of the PEG solution with 0.5 mL of the Tris buffer solution.

### Example 12: Viscosity Measurements of Formulations Containing Excipient Compounds and PEG

Viscosity measurements of the formulations prepared were made with a DV-IIT LV cone and plate viscometer (Brookfield Engineering, Middleboro, Mass.). The viscometer was equipped with a CP-40 cone and was operated at 3 rpm and 25° C. The formulation was loaded into the viscometer at a volume of 0.5 mL and allowed to incubate at the given shear rate and temperature for 3 minutes, followed by a measurement collection period of twenty seconds. This was then followed by 2 additional steps consisting of 1 minute of shear incubation and subsequent twenty second measurement collection period. The three data points collected were then averaged and recorded as the viscosity for the sample.

The results presented in Table 7 show the effect of the added excipient compounds in reducing viscosity.

TABLE 7

| | Excipient | | |
|---|---|---|---|
| | Concentration | Viscosity | Viscosity |
| Excipient | (mg/mL) | (cP) | Reduction |
| None | 0 | 104.8 | 0% |
| Citric acid Na salt | 40 | 56.8 | 44% |
| Citric acid Na salt | 20 | 73.3 | 28% |
| glycerol phosphate | 40 | 71.7 | 30% |
| glycerol phosphate | 20 | 83.9 | 18% |
| Ethylene diamine | 40 | 84.7 | 17% |
| Ethylene diamine | 20 | 83.9 | 15% |
| EDTA/K salt | 40 | 67.1 | 36% |
| EDTA/K salt | 20 | 76.9 | 27% |
| EDTA/Na salt | 40 | 68.1 | 35% |
| EDTA/Na salt | 20 | 77.4 | 26% |
| D-Gluconic acid/K salt | 40 | 80.32 | 23% |
| D-Gluconic acid/K salt | 20 | 88.4 | 16% |
| D-Gluconic acid/Na salt | 40 | 81.24 | 23% |
| D-Gluconic acid/Na salt | 20 | 86.6 | 17% |
| lactic acid/K salt | 40 | 80.42 | 23% |
| lactic acid/K salt | 20 | 85.1 | 19% |
| lactic acid/Na salt | 40 | 86.55 | 17% |
| lactic acid/Na salt | 20 | 87.2 | 17% |
| etidronic acid/K salt | 24 | 71.91 | 31% |
| etidronic acid/K salt | 12 | 80.5 | 23% |
| etidronic acid/Na salt | 24 | 71.6 | 32% |
| etidronic acid/Na salt | 12 | 79.4 | 24% |

### Example 13: Preparation of PEGylated BSA with 1 PEG Chain Per BSA Molecule

To a beaker was added 200 mL of a phosphate buffered saline (Aldrich Cat. #P4417) and 4 g of BSA (Aldrich Cat. #A7906) and mixed with a magnetic bar. Next 400 mg of methoxy polyethylene glycol maleimide, MW=5,000, (Aldrich Cat. #63187) was added. The reaction mixture was allowed to react overnight at room temperature. The following day, 20 drops of HCl 0.1 M were added to stop the reaction. The reaction product was characterized by SDS-Page and SEC which clearly showed the PEGylated BSA. The reaction mixture was placed in an Amicon centrifuge tube with a molecular weight cutoff (MWCO) of 30 kDa and concentrated to a few milliliters. Next the sample was diluted 20 times with a histidine buffer, 50 mM at a pH of approximately 6, followed by concentrating until a high viscosity fluid was obtained. The final concentration of the protein solution was obtained by measuring the absorbance at 280 nm and using a coefficient of extinction for the BSA of 0.6678. The results indicated that the final concentration of BSA in the solution was 342 mg/mL.

### Example 14: Preparation of PEGylated BSA with Multiple PEG Chains Per BSA Molecule

A 5 mg/mL solution of BSA (Aldrich A7906) in phosphate buffer, 25 mM at pH of 7.2, was prepared by mixing 0.5 g of the BSA with 100 mL of the buffer. Next 1 g of a methoxy PEG propionaldehyde Mw=20,000 (JenKem Technology, Plano, Tex. 75024) was added followed by 0.12 g of sodium cyanoborohydride (Aldrich 156159). The reaction was allowed to proceed overnight at room temperature. The following day the reaction mixture was diluted 13 times with a Tris buffer (10 mM Tris, 135 mM NaCl at pH=7.3) and concentrated using Amicon centrifuge tubes MWCO of 30 kDa until a concentration of approximately 150 mg/mL was reached.

### Example 15: Preparation of PEGylated Lysozyme with Multiple PEG Chains Per Lysozyme Molecule

A 5 mg/mL solution of lysozyme (Aldrich L6876) in phosphate buffer, 25 mM at pH of 7.2, was prepared by mixing 0.5 g of the lysozyme with 100 mL of the buffer. Next 1 g of a methoxy PEG propionaldehyde Mw=5,000 (JenKem Technology, Plano, Tex. 75024) was added followed by 0.12 g of sodium cyanoborohydride (Aldrich 156159). The reaction was allowed to proceed overnight at room temperature. The following day the reaction mixture was diluted 49 times with the phosphate buffer, 25 mM at pH of 7.2, and concentrated using Amicon centrifuge tubes MWCO of 30 kDa. The final concentration of the protein solution was obtained by measuring the absorbance at 280 nm and using a coefficient of extinction for the lysozyme of 2.63. The final concentration of lysozyme in the solution was 140 mg/mL.

### Example 16: Effect of Excipients on Viscosity of PEGylated BSA with 1 PEG Chain Per BSA Molecule

Formulations of PEGylated BSA (from Example 13 above) with excipients were prepared by adding 6 or 12 milligrams of the excipient salt to 0.3 mL of the PEGylated BSA solution. The solution was mixed by gently shaking and the viscosity was measured by a RheoSense microVisc equipped with an A10 channel (100-micron depth), and at a shear rate of 500 s⁻¹. The viscometer measurements were completed at ambient temperature. The results presented in Table 8 shows the effect of the added excipient compounds in reducing viscosity.

TABLE 8

| | Excipient | | |
|---|---|---|---|
| | Concentration | Viscosity | Viscosity |
| Excipient | (mg/mL) | (cP) | Reduction |
| None | 0 | 228.6 | 0% |
| Alpha-Cyclodextrin | 20 | 151.5 | 34% |
| sulfated Na salt | | | |
| K acetate | 40 | 89.5 | 60% |

### Example 17: Effect of Excipients on Viscosity of PEGylated BSA with Multiple PEG Chains Per BSA Molecule

A formulation of PEGylated BSA (from Example 14 above) with citric acid Na salt as excipient was prepared by adding 8 milligrams of the excipient salt to 0.2 mL of the PEGylated BSA solution. The solution was mixed by gently shaking and the viscosity was measured by a RheoSense microVisc equipped with an A10 channel (100 micron depth), and at a shear rate of 500 s⁻¹. The viscometer measurements were completed at ambient temperature. The results presented in Table 9 shows the effect of the added excipient compounds in reducing viscosity.

TABLE 9

| | Excipient | | |
|---|---|---|---|
| | Concentration | Viscosity | Viscosity |
| Excipient Added | (mg/mL) | (cP) | Reduction |
| None | 0 | 56.8 | 0% |
| Citric acid Na salt | 40 | 43.5 | 23% |

### Example 18: Effect of Excipients on Viscosity of PEGylated Lysozyme with Multiple PEG Chains Per Lysozyme Molecule

A formulation of PEGylated lysozyme (from Example 15 above) with potassium acetate as excipient was prepared by adding 6 milligrams of the excipient salt to 0.3 mL of the PEGylated lysozyme solution. The solution was mixed by gently shaking and the viscosity was measured by a RheoSense microVisc equipped with an A10 channel (100 micron depth) at a shear rate of 500 s⁻¹. The viscometer measurements were completed at ambient temperature. The results presented in the next table (Table 10) shows the benefit of the added excipient compounds in reducing viscosity.

TABLE 10

| | Excipient | | |
|---|---|---|---|
| | Concentration | Viscosity | Viscosity |
| Excipient | (mg/mL) | (cP) | Reduction |
| None | 0 | 24.6 | 0% |
| K acetate | 20 | 22.6 | 8% |

### Example 19: Protein Formulations Containing Excipient Combinations

Formulations were prepared using an excipient compound or a combination of two excipient compounds and a test protein, where the test protein was intended to simulate a therapeutic protein that would be used in a therapeutic formulation. These formulations were prepared in 20 mM histidine buffer with different excipient compounds for viscosity measurement in the following way. Excipient combinations were dissolved in 20 mM histidine and the resulting solution pH adjusted with small amounts of sodium hydroxide or hydrochloric acid to achieve pH 6 prior to dissolution of the model protein. Excipient compounds for this Example are listed below in Table 11. Once the excipient solutions had been prepared, the test protein bovine gamma globulin (BGG) was dissolved at a ratio to achieve a final protein concentration of about 280 mg/mL. Solutions of BGG in the excipient solutions were formulated in 5 mL sterile polypropylene tubes and allowed to shake at 80-100 rpm on an orbital shaker table overnight. BGG solutions were then transferred to 2 mL microcentrifuge tubes and centrifuged for about ten minutes at 2300 rpm in an IEC MicroMax microcentrifuge to remove entrained air prior to viscosity measurement.

Viscosity measurements of formulations prepared as described above were made with a DV-IIT LV cone and plate viscometer (Brookfield Engineering, Middleboro, Mass.). The viscometer was equipped with a CP-40 cone and was operated at 3 rpm and 25° C. The formulation was loaded into the viscometer at a volume of 0.5 mL and allowed to incubate at the given shear rate and temperature for 3 minutes, followed by a measurement collection period of twenty seconds. This was then followed by 2 additional steps consisting of 1 minute of shear incubation and subsequent twenty second measurement collection period. The three data points collected were then averaged and recorded as the viscosity for the sample. Viscosities of solutions with excipient were normalized to the viscosity of the model protein solution without excipient, and the results are shown in Table 11 below. The normalized viscosity is the ratio of the viscosity of the model protein solution with excipient to the viscosity of the model protein solution with no excipient.

TABLE 11

| Excipient A | | Excipient B | | |
|---|---|---|---|---|
| | Conc. | | Conc. | Normalized |
| Name | (mg/mL) | Name | (mg/mL) | Viscosity |
| None | 0 | None | 0 | 1.00 |
| Aspartame | 10 | None | 0 | 0.83 |
| Saccharin | 60 | None | 0 | 0.51 |
| Acesulfame K | 80 | None | 0 | 0.44 |
| Theophylline | 10 | None | 0 | 0.84 |
| Saccharin | 30 | None | 0 | 0.58 |
| Acesulfame K | 40 | None | 0 | 0.61 |
| Caffeine | 15 | Taurine | 15 | 0.82 |
| Caffeine | 15 | Tyramine | 15 | 0.67 |

### Example 20: Protein Formulations Containing Excipients to Reduce Viscosity and Injection Pain

Formulations were prepared using an excipient compound, a second excipient compound, and a test protein, where the test protein was intended to simulate a therapeutic protein that would be used in a therapeutic formulation. The first excipient compound, Excipient A, was selected from a group of compounds having local anesthetic properties. The first excipient, Excipient A and the second excipient, Excipient B are listed in Table 12. These formulations were prepared in 20 mM histidine buffer using Excipient A and Excipient B in the following way, so that their viscosities could be measured. Excipients in the amounts disclosed in Table 12 were dissolved in 20 mM histidine and the resulting solutions were pH adjusted with small amounts of sodium hydroxide or hydrochloric acid to achieve pH 6 prior to dissolution of the model protein. Once excipient solutions had been prepared, the test protein bovine gamma globulin (BGG) was dissolved in the excipient solution at a ratio to achieve a final protein concentration of about 280 mg/mL. Solutions of BGG in the excipient solutions were formulated in 5 mL sterile polypropylene tubes and allowed to shake at 80-100 rpm on an orbital shaker table overnight. BGG-excipient solutions were then transferred to 2 mL microcentrifuge tubes and centrifuged for about ten minutes at 2300 rpm in an IEC MicroMax microcentrifuge to remove entrained air prior to viscosity measurement.

Viscosity measurements of the formulations prepared as described above were made with a DV-IIT LV cone and plate viscometer (Brookfield Engineering, Middleboro, Mass.). The viscometer was equipped with a CP-40 cone and was operated at 3 rpm and 25° C. The formulation was loaded into the viscometer at a volume of 0.5 mL and allowed to incubate at the given shear rate and temperature for 3 minutes, followed by a measurement collection period of twenty seconds. This was then followed by 2 additional steps consisting of 1 minute of shear incubation and subsequent twenty second measurement collection period. The three data points collected were then averaged and recorded as the viscosity for the sample. Viscosities of solutions with excipient were normalized to the viscosity of the model protein solution without excipient, and the results are shown in Table 12 below. The normalized viscosity is the ratio of the viscosity of the model protein solution with excipient to the viscosity of the model protein solution with no excipient.

TABLE 12

| Excipient A | | Excipient B | | |
|---|---|---|---|---|
| | Conc. | | Conc. | Normalized |
| Name | (mg/mL) | Name | (mg/mL) | Viscosity |
| None | 0 | None | 0 | 1.00 |
| Lidocaine | 45 | None | 0 | 0.73 |
| Lidocaine | 23 | None | 0 | 0.74 |
| Lidocaine | 10 | Caffeine | 15 | 0.71 |
| Procaine HCl | 40 | None | 0 | 0.64 |
| Procaine HCl | 20 | Caffeine | 15 | 0.69 |

### Example 21: Formulations Containing Excipient Compounds and PEG

Formulations were prepared using an excipient compound and PEG, where the PEG was intended to simulate a therapeutic PEGylated protein that would be used in a therapeutic formulation, and where the excipient compounds were provided in the amounts as listed in Table 13. These formulations were prepared by mixing equal volumes of a solution of PEG with a solution of the excipient. Both solutions were prepared in deionized (DI) Water. The PEG solution was prepared by mixing 16.5 g of poly(ethylene oxide) average Mw ˜100,000 (Aldrich Catalog #181986) with 83.5 g of DI water. The mixture was stirred overnight for complete dissolution.

The excipient solutions were prepared by this general method and as detailed in Table 13 below: An approximately 20 mg/mL solution of potassium phosphate tribasic (Aldrich Catalog #P5629) in DI water was prepared by dissolving 0.05 g of potassium phosphate in 5 mL of DI water. The PEG excipient solution was prepared by mixing 0.5 mL of the PEG solution with 0.5 mL of the excipient solution and mixed by using a vortex for a few seconds. A control sample was prepared by mixing 0.5 mL of the PEG solution with 0.5 mL of DI water. Viscosity was measured and results are recorded in Table 13 below.

TABLE 13

| | Excipient | | Viscosity |
|---|---|---|---|
| | Concentration | Viscosity | Reduction |
| Excipient | (mg/mL) | (cP) | (%) |
| None | 0 | 79.7 | 0 |
| Citric acid Na salt | 10 | 74.9 | 6.0 |
| Potassium phosphate | 10 | 72.3 | 9.3 |
| Citric acid Na salt/ | 10/10 | 69.1 | 13.3 |
| Potassium phosphate | | | |
| Sodium sulfate | 10 | 75.1 | 5.8 |
| Citric acid Na salt/ | 10/10 | 70.4 | 11.7 |
| Sodium sulfate | | | |

### Example 22: Improved Processing of Protein Solutions with Excipients

Two BGG solutions were prepared by mixing 0.25 g of solid BGG with 4 mL of a buffer solution. For Sample A: Buffer solution was 20 mM histidine buffer (pH=6.0). For sample B: Buffer solution was 20 mM histidine buffer containing 15 mg/mL of caffeine (pH=6). The dissolution of the solid BGG was carried out by placing the samples in an orbital shaker set at 100 rpm. The buffer sample containing caffeine excipient was observed to dissolve the protein faster. For the sample with the caffeine excipient (Sample B) complete dissolution of the BGG was achieved in 15 minutes. For the sample without the caffeine (Sample A) the dissolution needed 35 minutes. Next, the samples were placed in 2 separate Amicon Ultra 4 Centrifugal Filter Unit with a 30 kDa molecular weight cut off and the samples were centrifuged at 2,500 rpm at 10 minutes intervals. The filtrate volume recovered after each 10 minute centrifuge run was recorded. The results in Table 14 show the faster recovery of the filtrate for Sample B. In addition, Sample B kept concentrating with every additional run but Sample A reached a maximum concentration point and further centrifugation did not result in further sample concentration.

TABLE 14

| Centrifuge time | Sample A filtrate | Sample B filtrate |
|---|---|---|
| (min) | collected (mL) | collected (mL) |
| 10 | 0.28 | 0.28 |
| 20 | 0.56 | 0.61 |
| 30 | 0.78 | 0.88 |
| 40 | 0.99 | 1.09 |
| 50 | 1.27 | 1.42 |
| 60 | 1.51 | 1.71 |
| 70 | 1.64 | 1.99 |
| 80 | 1.79 | 2.29 |
| 90 | 1.79 | 2.39 |
| 100 | 1.79 | 2.49 |

### Example 23: Protein Formulations Containing Multiple Excipients

This example shows how the combination of caffeine and arginine as excipients has a beneficial effect on decreasing viscosity of a BGG solution. Four BGG solutions were prepared by mixing 0.18 g of solid BGG with 0.5 mL of a 20 mM Histidine buffer at pH 6. Each buffer solution contained different excipient or combination of excipients as described in the table below (Table 15). The viscosity of the solutions was measured as described in previous examples. The results show that the hindered amine excipient, caffeine, can be combined with known excipients such as arginine, and the combination has better viscosity reduction properties than the individual excipients by themselves.

TABLE 15

| | | | Viscosity |
|---|---|---|---|
| | | Viscosity | Reduction |
| Sample | Excipient(s) added | (cP) | (%) |
| A | None | 130.6 | 0 |
| B | Caffeine (10 mg/mL) | 87.9 | 33 |
| C | Caffeine (10 mg/mL)/ | 66.1 | 49 |
| | Arginine (25 mg/mL) | | |
| D | Arginine (25 mg/mL) | 76.7 | 41 |

Arginine was added to 280 mg/mL solutions of BGG in histidine buffer at pH 6. At levels above 50 mg/mL, adding more arginine did not decrease viscosity further, as shown in Table 16.

TABLE 16

| Arginine added | Viscosity | Viscosity reduction |
|---|---|---|
| (mg/mL) | (cP) | (%) |
| 0 | 79.0 | 0% |
| 53 | 40.9 | 48% |
| 79 | 46.1 | 42% |
| 105 | 47.8 | 40% |
| 132 | 49.0 | 38% |
| 158 | 48.0 | 39% |
| 174 | 50.3 | 36% |
| 211 | 51.4 | 35% |

Caffeine was added to 280 mg/mL solutions of BGG in histidine buffer at pH 6. At levels above 10 mg/mL, adding more caffeine did not decrease viscosity further, as shown in Table 17.

TABLE 17

| Caffeine added | Viscosity | Viscosity reduction |
|---|---|---|
| (mg/mL) | (cP) | (%) |
| 0 | 79 | 0% |
| 10 | 60 | 31% |
| 15 | 62 | 23% |
| 22 | 50 | 45% |

### Example 24: Caffeine Effect During TFF Concentration Process

In this Example, bovine gamma globulin (BGG) solutions were concentrated in the presence and absence of caffeine using tangential flow filtration (TFF). The Labscale TFF System, produced by EMVD Millipore (Billerica, Mass.) was used to perform the experiments. The system was fitted with a Pellicon XL TFF cassette that contained an Ultracel membrane with 30 kDa molecular weight cutoff (EMD Millipore, Billerica, Mass.). The nominal membrane surface area was 50 cm². The feed pressure to the cassette was maintained at 30 psi while the retentate pressure was maintained at 10 psi. The filtrate flux was monitored over the course of the experiment by measuring its mass as a function of time. Approximately 12 grams of BGG were dissolved into 500 mL of buffer containing 15 mg/mL caffeine, 150 mM NaCl, and 20 mM histidine, adjusted to pH 6. A control sample was prepared by dissolving 12 grams of BGG into 500 mL of buffer containing 150 mM NaCl, and 20 mM histidine, adjusted to pH 6. The buffer components were purchased from Sigma-Aldrich. Both solutions were filtered through a 0.2 μm polyethersulfone (PES) filter (VWR, Radnor, Pa.) prior to TFF processing. The performance of the test sample and control sample during TFF were measured by the mass transfer coefficient. The mass transfer coefficient was determined for each sample using the following equation (as described in J. Hung, A. U. Borwankar, B. J. Dear, T. M. Truskett, K. P. Johnston, High concentration tangential flow ultrafiltration of stable monoclonal antibody solutions with low viscosities. J. Memb. Sci. 508, 113-126 (2016)):

J=k_{c} ln(C_{w}/C_{b}) (Eq. 3)

Eq. 3 describes the filtrate flux J, where k_{c} is the mass transfer coefficient, C_{w} is the protein concentration in the vicinity of the membrane, and C_{b} is the concentration in the liquid bulk, and Eq. 3 thereby permits calculation of the mass transfer coefficient k_{c}. A graph of the calculated flux J against the ln(C_{b}) yields a linear plot with slope of −kc. Here the flux J is calculated by taking the derivative of the filtrate mass with respect to time and C_{b} is calculated using a mass-balance. The best-fit mass transfer coefficients are listed in Table 18. The introduction of 15 mg/mL caffeine increased the value of the mass transfer coefficient by ˜13%, from 22.5 to 25.4 Lm⁻² hr⁻¹ (LMH).

TABLE 18

| Sample | Mass Transfer Coefficient k_{c} (LMH) |
|---|---|
| Control | 22.5 ± 0.1 |
| 15 mg/mL caffeine | 25.4 ± 0.1 |

### Example 25: Caffeine Effect During TFF Concentration Process

In this Example, bovine gamma globulin (BGG) solutions were concentrated in the presence and absence of caffeine using tangential flow filtration (TFF). The Labscale TFF System, produced by EMD Millipore (Billerica, Mass.) was used to perform the experiments. The system was fitted with a Pellicon XL TFF cassette that contained an Ultracel membrane with 30 kDa molecular weight cutoff (EMD Millipore, Billerica, Mass.). The nominal membrane surface area was 50 cm². A control sample was prepared by dissolving 14.6 grams of BGG into 582 mL of buffer containing 150 mM NaCl, and 20 mM histidine, adjusted to pH 6, such that the initial BGG concentration was nominally 25.1 mg/mL. The material was filtered through a 0.2 μm PES filter (VWR, Radnor, Pa.) and then processed in the TFF device. The pump speed was adjusted such that the feed pressure was initially 30 psi and the retentate valve was adjusted such that the retentate pressure was initially 10 psi. The material was concentrated without adjusting either the pump speed or retentate valve for 4.1 hours. The initial and final concentrations were determined to be 25.4±0.6 and 159±6 mg/mL, respectively, by a Bradford assay, as shown in Table 19 below. A caffeine-containing sample was prepared by dissolving 14.2 g of BGG into 566 mL of buffer containing 15 mg/mL caffeine, 150 mM NaCl, and 20 mM histidine, adjusted to pH 6, such that the initial BGG concentration was nominally 25.1 mg/mL. The material was filtered through a 0.2 μm PES filter (VWR, Radnor, Pa.) and then processed in the TFF device. The pump speed and retentate valve were set to identical levels to those previously. The feed and retentate pressures were confirmed to be 30 psi and 10 psi, respectively, as previously. The material was concentrated without adjusting either the pump speed or retentate valve for 4.1 hours. The initial and final concentrations were determined to be 24.4±0.5 and 225±10 mg/mL, respectively, by a Bradford assay, as shown in Table 19 below. The use of caffeine during TFF processing increased the final protein concentration by approximately 42% when compared to the control, from 159 to 225 mg/mL.

TABLE 19

| | Initial concentration | Final concentration |
|---|---|---|
| Sample | (mg/mL) | (mg/mL) |
| Control | 25.4 ± 0.6 | 159 ± 6 |
| 15 mg/mL caffeine | 24.4 ± 0.5 | 225 ± 10 |

### Example 26: Caffeine Effect During Sterile Filtration of BGG Solutions

Bovine gamma globulin (BGG), L-histidine, and caffeine were purchased from Sigma-Aldrich (St. Louis, Mo., product numbers G5009, H6034, and C7731, respectively). Deionized (DI) water was generated from tap water with a Direct-Q 3 UV purification system from EMD Millipore (Billerica, Mass.). 25-mm polyethersulfone (PES) filters with 0.2-μm pores were purchased from GE Healthcare (Chicago, Ill., catalog number 6780-2502). 1-mL Luer-Lok syringes were purchased from Becton, Dickinson and Company (Franklin Lakes, N.J., reference number 309628). A 20-mM histidine buffer, pH 6.0 was prepared using L-histidine, DI water, and titrated to pH 6.0 with 1 M HCl. A 15 mg/mL solution of caffeine was prepared using the histidine buffer. The caffeine-free and caffeine-containing buffers were used to reconstitute BGG to a final concentration of about 280 mg/mL. The protein concentration, c, was calculated using:

$$c = \frac{{m}_{p}}{b + {vm}_{p}} \qquad ( Eq . 4 )$$

where mₚ is the protein mass, b is the volume of buffer added, and v is the partial specific volume of BGG, here taken to be 0.74 mL/g. The viscosity of each sample was measured using microVisc rheometer (RheoSense, San Ramon, Calif.) at a temperature of 23° C. and shear rate of 250 s⁻¹. The energies required to pass the BGG solutions through the sterile filters were measured using a Tensile Compression Tester (TCT, Instron, Needham, Mass., part number 3343) fitted with a 100 N load cell (Instron, Needham, Mass., part number 2519-103). The syringe plungers were depressed at a rate of 159 mm/min for a distance of 50 mm. The energy requirements were calculated by integrating the load-versus-extension curves measured by the TCT, and results are summarized in Table 20 below.

TABLE 20

| | Protein | Caffeine | | Energy |
|---|---|---|---|---|
| | concentration | concentration | Viscosity | requirement |
| Sample | (mg/mL) | (mg/mL) | (cP) | (mJ) |
| 1 | 280 | 0 | 106 | 198 |
| 2 | 280 | 15.1 | 68.9 | 181 |

### Example 27: Excipients to Improve Protein-A Chromatography Elution

Four purified, research-grade biosimilar antibodies, ipilimumab, ustekinumab, omalizumab, and tocilizumab were purchased from Bioceros (Utrecht, The Netherlands). They were provided as frozen aliquots at protein concentrations of 20, 26, 15 and 23 mg/mL, respectively, in an aqueous 40 mM sodium acetate, 50 mM tris-HCl buffer at pH 5.5. The protein solutions were thawed at room temperature prior to measurement and afterwards, were filtered through a 0.2 μm polyethersulfone filters. The filtered protein stock solutions were mixed in 1:1 ratio of protein stock solution to a binding buffer. The binding buffer, used to promote the binding of the antibodies to the Protein-A resin, was composed of 0.1 M sodium phosphate and 0.15 sodium chloride at pH 7.2 in deionized (DI) water. The DI water was produced by purifying tap water with a Direct-Q 3 UV purification system from EMD Millipore (Billerica, Mass.). These solutions were employed to perform Protein-A binding and elution studies using a Pierce™ Protein-A Spin Plate for IgG Screening (ThermoFisher Scientific catalog #45202). The plate had 96 wells, each containing 50 μL of Protein-A resin. The resin was washed with binding buffer by adding 200 μL of binding buffer to each well and centrifuging the plate at 1000×g for 1 minute and discarding the flow-through. All subsequent centrifugation steps were performed at 1000×g for 1 minute. This wash procedure was repeated once. Following these initial washing steps, the diluted protein samples, i.e., samples containing ipilimumab, ustekinumab, omalizumab, and tocilizumab, were added to the wells in the plate (200 μL per well). The plate was then placed on a Daigger Scientific (Vernon Hills, Ill.) Labgenius orbital shaker and agitated at 260 rpm for 30 minutes, following which the plate was centrifuged and the flow-through was discarded. The wells were then washed by adding 500 μL of binding buffer to each well, centrifuging the plate and discarding the flow-through. This wash step was repeated twice. After these washing steps, the proteins were eluted from the plate using elution buffers to which different excipients had been added. For each elution, 50 μL of a neutralization buffer consisting of 1 M sodium phosphate at pH 7 was added to each well of the collection plate, and then two hundred μL of elution buffer was added to each well of the plate. The plate was agitated at 260 rpm for 1 minute and then centrifuged. The flow-through was recovered for analysis. This elution step was repeated once. The control buffer, with no excipients, contained 20 mM citrate and had a pH of 2.6. Because Protein-A elution buffers often contain some amount of salt, an elution buffer of 100 mM NaCl in the citrate buffer was prepared as a secondary control.

Table 21 lists the excipient solutions used in this example, their concentrations, and final pH of the elution buffers. All excipients were purchased from Sigma Aldrich (St. Louis, Mo.), with the exception of aspartame, which was purchased from Herb Store USA (Los Angeles, Calif.), trehalose, which was purchased from Cascade Analytical Reagents and Biochemicals (Corvallis, Oreg.), and sucrose which was purchased from Research Products International (Mt. Prospect, Ill., product number S24060). All excipient-containing elution buffers were prepared by mixing the appropriate quantity of the excipient with approximately 10 mL of the salt-free citrate buffer control. The elution buffers were prepared at approximately 100 mM excipient. However, not all of the excipients are soluble at this level; Table 21 therefore lists all of the excipient concentrations that were used. The pH of each elution buffer was adjusted to about 2.6±0.1 using either hydrochloride or sodium hydroxide as needed.

For each protein sample, ASD High performance size-exclusion chromatography (SEC) analysis was performed using a TSKgel SuperSW3000 column (30 cm×4.6 mm ID, Tosoh Bioscience, King of Prussia, Pa.) connected to an HPLC workstation (Agilent HP 1100 system). The separation was carried out at a flow of 0.35 mL/min at room temperature. The mobile phase was an aqueous buffer of 100 mM sodium phosphate, 300 mM sodium chloride, pH 7. The protein concentration was monitored by absorbance at 280 nm using an Agilent 1100 Series G1315B diode array detector. The total amount of protein eluted from the Protein-A resin for each protein, i.e., ipilimumab, ustekinumab, omalizumab, and tocilizumab, was estimated by integrating the chromatograms. The integrated peak areas for each protein, i.e., ipilimumab, ustekinumab, omalizumab, and tocilizumab, are listed in Tables 22-25. Tables 22-25 also compare the experimental peak areas to those of the salt-free and salt-containing controls. Values greater than 100% indicate that the elution buffer recovered more protein from the Protein-A resin than the control whereas values less than 100% indicate that the elution buffer recovered less protein from the Protein-A resin than the control.

TABLE 21

| | Sigma-Aldrich | Excipient | |
|---|---|---|---|
| | product number | concentration | |
| Excipient | for excipient | (mM) | pH |
| Caffeine | C7731 | 79 | 2.6 |
| acesulfame | 04054 | 110 | 2.5 |
| potassium | | | |
| 1-methyl-2- | M6762 | 117 | 2.6 |
| pyrrolidone | | | |
| aspartame | N/A | 20 | 2.6 |
| taurine | T8691 | 114 | 2.5 |
| trehalose | N/A | 100 | 2.7 |
| sucrose | N/A | 101 | 2.7 |
| niacinamide | N5535 | 99 | 2.7 |
| sodium chloride | S7653 | 117 | 2.6 |
| control | | | |
| Control | N/A | N/A | 2.5 |

TABLE 22
Ipilimumab recovery from Protein-A resin

| | | Peak area | Peak area |
|---|---|---|---|
| | Peak area | normalized to salt- | normalized to |
| Excipient | (mAU*min) | free control (%) | salt control (%) |
| Citrate | 3409 | 77.9 | 83.6 |
| Acesulfame | 1567 | 35.8 | 38.4 |
| potassium | | | |
| 1-methyl-2- | 386 | 8.8 | 9.5 |
| pyrrolidone | | | |
| Aspartame | 4012 | 91.7 | 98.3 |
| Taurine | 3958 | 90.4 | 97.0 |
| Trehalose | 3667 | 83.8 | 89.9 |
| Sucrose | 4585 | 104.8 | 112.4 |
| Niacinamide | 4295 | 98.2 | 105.3 |
| sodium chloride | 4080 | 93.2 | 100.0 |
| control | | | |
| Control | 4376 | 100.0 | 107.2 |

TABLE 23
Ustekinumab recovery from Protein-A resin

| | | Peak area | Peak area |
|---|---|---|---|
| | Integrated | normalized to | normalized |
| | peak area | salt-free | to salt |
| Excipient | (mAU*min) | control (%) | control (%) |
| Caffeine | 2301 | 86.6 | 75.2 |
| acesulfame | 307 | 16.2 | 14.0 |
| potassium | | | |
| Aspartame | 417 | 17.4 | 15.1 |
| 1-methyl-2- | 2952 | 108.8 | 94.4 |
| pyrrolidone | | | |
| Taurine | 3257 | 118.6 | 103.0 |
| Trehalose | 1549 | 56.6 | 49.1 |
| sucrose | 1274 | 51.2 | 44.4 |
| niacinamide | 3204 | 116.1 | 100.8 |
| sodium | 3176 | 115.2 | 100.0 |
| chloride | | | |
| control | | | |

TABLE 24
Omalizumab recovery from Protein-A resin

| | | Peak area | Peak area |
|---|---|---|---|
| | Integrated | normalized to | normalized |
| | peak area | salt-free | to salt |
| Excipient | (mAU*min) | control (%) | control (%) |
| caffeine | 4040 | 105.5 | 117.5 |
| acesulfame | 3620 | 94.5 | 105.3 |
| potassium | | | |
| 1-methyl-2- | 3334 | 87.0 | 97.0 |
| pyrrolidone | | | |
| aspartame | 3605 | 94.1 | 104.8 |
| taurine | 4337 | 113.2 | 126.1 |
| trehalose | 3571 | 93.2 | 103.8 |
| sucrose | 3639 | 95.0 | 105.8 |
| niacinamide | 4812 | 125.6 | 139.9 |
| sodium chloride | 3439 | 89.8 | 100.0 |
| control | | | |
| control | 3831 | 100.0 | 111.4 |

TABLE 25
Tocilizumab recovery from Protein-A resin

| | | Peak area | Peak area |
|---|---|---|---|
| | Integrated | normalized to | normalized |
| | peak area | salt-free | to salt |
| Excipient | (mAU*min) | control (%) | control (%) |
| caffeine | 3120 | 111.2 | 100.3 |
| acesulfame | 3083 | 109.9 | 99.1 |
| potassium | | | |
| 1-methyl-2- | 261 | 9.3 | 8.4 |
| pyrrolidone | | | |
| aspartame | 556 | 19.8 | 17.9 |
| taurine | 3054 | 108.8 | 98.2 |
| trehalose | 2781 | 99.1 | 89.4 |
| sucrose | 1037 | 37.0 | 33.3 |
| niacinamide | 2550 | 90.9 | 82.0 |
| sodium chloride | 3111 | 110.9 | 100.0 |
| control | | | |
| control | 2806 | 100.0 | 90.2 |

### Example 28: Excipients to Improve Protein-A Chromatography Elution

The test proteins used in this Example are identical to those in Example 27, i.e., ipilimumab, ustekinumab, omalizumab, and tocilizumab. Protein-A binding and elution studies were performed using an identical plate to that in Example 27. The methods for loading and eluting the antibodies from the Protein-A plate were identical to those in Example 27 with the exception of the elution step. In Example 27, two elution washes were performed. However, in this Example, only one wash is performed. As in Example 27, elution buffers were prepared from a 20 mM citrate, pH 2.6 control buffer. The excipients are listed in Table 26 below. All of the excipients were purchased from Sigma-Aldrich (St. Louis, Mo.). The recovered protein was analyzed by HPLC in an identical fashion to that in Example 27, and results of protein recovery for each protein, i.e., ipilimumab, ustekinumab, omalizumab, and tocilizumab, are documented in Tables 26-30 below.

TABLE 26
Excipients used in Example 28

| | | Excipient | |
|---|---|---|---|
| | Sigma-Aldrich | concentration | |
| Excipient | product number | (mM) | pH |
| control | N/A | N/A | 2.5 |
| sodium chloride control | S7653 | 117 | 2.6 |
| niacinamide | N5535 | 99 | 2.7 |
| taurine | T8691 | 114 | 2.5 |
| imidazole | I5513 | 100 | 2.6 |
| 4-hydroxybenzesulfonic | 171506 | 107 | 2.6 |
| acid | | | |
| caffeine | C7731 | 79 | 2.6 |

TABLE 27
Ipilimumab recovery from Protein-A resin

| | | Peak area | Peak area |
|---|---|---|---|
| | | normalized to | normalized |
| | Peak area | salt-free | to salt |
| Excipient | (mAU*min) | control (%) | control (%) |
| control | 4841 | 100.0 | 88.3 |
| sodium chloride control | 5485 | 113.3 | 100.0 |
| niacinamide | 6300 | 130.1 | 114.8 |
| taurine | 7557 | 156.1 | 137.8 |
| imidazole | 6071 | 125.4 | 110.7 |
| 4-hydroxybenzesulfonic | 5836 | 120.6 | 106.4 |
| acid | | | |
| caffeine | 6051 | 125.0 | 110.3 |

TABLE 28
Ustekinumab recovery from Protein-A resin

| | | Peak area | Peak area |
|---|---|---|---|
| | Integrated | normalized to | normalized |
| | peak area | salt-free | to salt |
| Excipient | (mAU*min) | control (%) | control (%) |
| control | 4572 | 100.0 | 107.9 |
| sodium chloride control | 4238 | 92.7 | 100.0 |
| niacinamide | 5848 | 127.9 | 138.0 |
| taurine | 4744 | 103.8 | 112.0 |
| imidazole | 4617 | 101.0 | 108.9 |
| 4-hydroxybenzesulfonic | 4132 | 90.4 | 97.5 |
| acid | | | |
| caffeine | 5084 | 111.2 | 120.0 |

TABLE 29
Omalizumab recovery from Protein-A resin

| | | Peak area | Peak area |
|---|---|---|---|
| | Integrated | normalized to | normalized |
| | peak area | salt-free | to salt |
| Excipient | (mAU*min) | control (%) | control (%) |
| control | 4194 | 100.0 | 91.7 |
| sodium chloride control | 4574 | 109.1 | 100.0 |
| niacinamide | 5748 | 137.0 | 125.7 |
| taurine | 4676 | 111.5 | 102.2 |
| imidazole | 2589 | 61.7 | 56.6 |
| 4-hydroxybenzesulfonic | 3190 | 76.1 | 69.7 |
| acid | | | |
| caffeine | 5807 | 138.5 | 127.0 |

TABLE 30
Tocilizumab recovery from Protein-A resin

| | | Peak area | Peak area |
|---|---|---|---|
| | Integrated | normalized to | normalized |
| | peak area | salt-free | to salt |
| Excipient | (mAU*min) | control (%) | control (%) |
| control | 4667 | 100.0 | 97.5 |
| sodium chloride control | 4786 | 102.6 | 100.0 |
| niacinamide | 5225 | 111.9 | 109.2 |
| taurine | 5396 | 115.6 | 112.7 |
| imidazole | 4754 | 101.9 | 99.3 |
| 4-hydroxybenzesulfonic | 4539 | 97.3 | 94.8 |
| acid | | | |
| caffeine | 5656 | 121.2 | 118.2 |

### Example 29: Excipients that Improve Omalizumab Elution from Protein-A Chromatography Column

Research-grade omalizumab was purchased from Bioceros (Utrecht, The Netherlands) and provided frozen at 15 mg/mL in an aqueous 40 mM sodium acetate, 50 mM tris-HCl buffer, pH 5.5. The protein was thawed at room temperature prior to experiments and filtered through a 0.2 μm polyethersulfone filter. The filtered material was mixed in a 1:1 ratio with a binding buffer that consisted of 20 mM sodium phosphate, pH 7 in DI water. Tap water was purified with a Direct-Q 3 UV purification system from EMD Millipore (Billerica, Mass.) to produce the DI water. Protein-A purification was performed using a HiTrap Protein-A HP 1 mL column from GE Healthcare (Chicago, Ill., product number 29048576). For each experiment, the column was first equilibrated with 10 mL of binding buffer. Following equilibration, 30 mg of protein were loaded onto the Protein-A column. The column was then washed with 5 mL of binding buffer. After washing the column, bound omalizumab was eluted from the column using fractions of one of the elution buffers containing the excipients listed in Table 31 below. The elution buffers were prepared by dissolving the indicated excipients in a 20 mM citrate buffer, pH 4.0. All elution buffers were adjusted to pH 4.0. Five 1-mL fractions were collected. Finally, Protein-A was regenerated by washing the column with 5 mL of 100 mM citrate, pH 3.0 buffer. The flowrate for each step was 1 mL/min, which was maintained by a Fusion 100 infusion pump (Chemyx, Stafford, Tex.). 10-mL NormJect Luer Lok syringes were used (Henke Sass Wolf, Tuttlingen, Germany, reference number 4100-OOOVO).

Elution fractions, E1, E2, E3, E4, and E5, were assayed for total protein content by high performance size-exclusion chromatography (SEC) analysis. SEC analysis was performed using a TSKgel SuperSW3000 column (30 cm×4.6 mm ID, Tosoh Bioscience, King of Prussia, Pa.) connected to an HPLC workstation (Agilent HP 1100 system). The separation was carried out at a flow of 0.35 mL/min at room temperature. The mobile phase was an aqueous buffer of 100 mM sodium phosphate, 300 mM sodium chloride, pH 7. The protein concentration was monitored by absorbance at 280 nm using an Agilent 1100 Series G1315B diode array detector. The total amount of protein eluted from the Protein-A resin was estimated by integrating the chromatograms.

Citrate is a common excipient used in Protein-A chromatography and was therefore used here as a control. The eluate fractions for the control sample exhibited insoluble aggregates on storage overnight at 4° C. as evidenced by the formation of a precipitate phase. Therefore, the peak areas reported in Table 31 below represent the total soluble protein amounts in the eluate fractions. We note that insoluble aggregates were only observed in the control sample and none of the other samples exhibited such aggregates. Peak areas greater than that of the control (using the citrate excipient) indicate that the use of the test excipient can enable a more efficient separation of protein from the column.

TABLE 31

| | Elution | | | | | | |
|---|---|---|---|---|---|---|---|
| | excipient | E1 peak | E2 peak | E3 peak | E4 peak | E5 peak | Total |
| Elution | concentration | area | area | area | area | area | peak area |
| excipient | (mM) | (mAU * min) | (mAU * min) | (mAU * min) | (mAU * min) | (mAU * min) | (mAU * min) |
| citrate | 103 | 352 | 9670 | 4098 | 4245 | 2953 | 21318 |
| (control) | | | | | | | |
| imidazole | 100 | 236 | 10224 | 7373 | 3894 | 2620 | 24348 |
| taurine | 125 | 408 | 17018 | 7676 | 3349 | 2211 | 30662 |
| niacinamide | 102 | 228 | 14492 | 5307 | 2914 | 2014 | 24955 |
| caffeine | 81 | 617 | 21965 | 8069 | 3301 | 1911 | 35863 |

### Example 30: Formulations of BGG with Different Amounts of Caffeine Excipient

Formulations were prepared with different molar concentrations of caffeine (at concentrations listed in Table 32 below) and a test protein, where the test protein was intended to simulate a therapeutic protein that would be used in a therapeutic formulation. The formulations for this Example were prepared in 20 mM histidine buffer for viscosity measurement in the following way. Stock solutions of 0 and 80 mM caffeine were prepared in 20 mM histidine and the resulting solution pH adjusted with small amounts of sodium hydroxide or hydrochloric acid to achieve pH 6 prior to dissolution of the model protein. Additional solutions at various caffeine concentrations were prepared by blending the two stock solutions at various volume ratios, to provide a series of caffeine-containing solutions, at concentrations listed in Table 32 below. Once these excipient solutions had been prepared, the test protein bovine gamma globulin (BGG) was dissolved into each test solution at a ratio to achieve a final protein concentration of about 280 mg/mL by adding 0.7 mL of each excipient solution to 0.25 g lyophilized BGG powder. The BGG-containing solutions were formulated in 5 mL sterile polypropylene tubes and allowed to shake at 100 rpm on an orbital shaker table overnight. These solutions were then transferred to 2 mL microcentrifuge tubes and centrifuged for about five minutes at 2400 rpm in an IEC MicroMax microcentrifuge to remove entrained air prior to viscosity measurement.

Viscosity measurements of formulations prepared as described above were made with a microVisc viscometer (RheoSense, San Ramon, Calif.). The viscometer was equipped with an A-10 chip having a channel depth of 100 microns, and was operated at a shear rate of 250 s⁻¹ and 25° C. To measure viscosity, the test formulation was loaded into the viscometer, taking care to remove all air bubbles from the pipet. The pipet containing the loaded sample formulation was placed in the instrument and allowed to incubate at the measurement temperature for about five minutes. The instrument was then run until the channel was fully equilibrated with the test fluid, indicated by a stable viscosity reading, and then the viscosity recorded in centipoise. Viscosity results that were obtained are presented in Table 32 below.

TABLE 32

| Caffeine conc | | |
|---|---|---|
| (mM) | Viscosity (cP) | Normalized Viscosity |
| 0 | 83 | 1.00 |
| 5 | 67 | 0.81 |
| 10 | 70 | 0.84 |
| 20 | 77 | 0.92 |
| 30 | 63 | 0.76 |
| 40 | 65 | 0.78 |
| 50 | 65 | 0.78 |
| 60 | 57 | 0.69 |
| 70 | 50 | 0.60 |
| 80 | 50 | 0.60 |

### Example 31: Preparation of Solutions of Co-Solutes in Deionized Water

Compounds used as co-solutes to increase caffeine solubility in water were obtained from Sigma-Aldrich (St. Louis, Mo.) and included niacinamide, proline, procaine HCl, ascorbic acid, 2,5-dihydroxybenzoic acid, lidocaine, saccharin, acesulfame K, tyramine, and aminobenzoic acid. Solutions of each co-solute were prepared by dissolving dry solid in deionized water and in some cases adjusting the pH to a value between pH of about 6 and pH of about 8 with 5 M hydrochloric acid or 5 M sodium hydroxide as necessary. Solutions were then diluted to a final volume of either 25 mL or 50 mL using a Class A volumetric flask and concentration recorded based on the mass of compound dissolved and the final volume of the solution. Prepared solutions were used either neat or diluted with deionized water.

### Example 32: Caffeine Solubility Testing

The impact of different co-solutes on the solubility of caffeine at ambient temperature (about 23° C.) was assessed in the following way. Dry caffeine powder (Sigma-Aldrich, St. Louis, Mo.) was added to 20 mL glass scintillation vials and the mass of caffeine recorded. 10 mL of a co-solute solution prepared in accordance with Example 31 was added to the caffeine powder in certain cases; in other cases, a blend of a co-solute solution and deionized water was added to the caffeine powder, maintaining a final addition volume of 10 mL. The volume contribution of the dry caffeine powder was assumed to be negligible in any of these mixtures. A small magnetic stir bar was added to the vial, and the solution was allowed to mix vigorously on a stir plate for about 10 minutes. After about 10 minutes the vial was observed for dissolution of the dry caffeine powder, and the results are given in Table 33 below. These observations indicated that niacinamide, procaine HCl, 2,5-dihydroxybenzoic acid sodium salt, saccharin sodium salt, and tyramine chloride salt all enabled dissolution of caffeine to at least about four times the reported caffeine solubility limit (˜16 mg/mL at room temperature according to Sigma-Aldrich).

TABLE 33

| | Co-solute | | | |
|---|---|---|---|---|
| | | Conc. | Caffeine | |
| Test No. | Name | (mg/mL) | (mg/mL) | Observation |
| 33.1 | Proline | 100 | 50 | DND |
| 33.2 | Niacinamide | 100 | 50 | CD |
| 33.3 | Niacinamide | 100 | 60 | CD |
| 33.4 | Niacinamide | 100 | 75 | CD |
| 33.5 | Niacinamide | 100 | 85 | CD |
| 33.6 | Niacinamide | 100 | 100 | CD |
| 33.7 | Niacinamide | 80 | 85 | CD |
| 33.8 | Niacinamide | 50 | 80 | CD |
| 33.9 | Procaine HCl | 100 | 85 | CD |
| 33.10 | Procaine HCl | 50 | 80 | CD |
| 33.11 | Niacinamide | 30 | 80 | DND |
| 33.12 | Procaine HCl | 30 | 80 | DND |
| 33.13 | Niacinamide | 40 | 80 | MD |
| 33.14 | Procaine HCl | 40 | 80 | DND |
| 33.15 | Ascorbic acid, Na | 50 | 80 | DND |
| 33.16 | Ascorbic acid, Na | 100 | 80 | DND |
| 33.17 | 2,5 DHBA, Na | 40 | 80 | CD |
| 33.18 | 2,5 DHBA, Na | 20 | 80 | MD |
| 33.19 | Lidocaine HCl | 40 | 80 | DND |
| 33.20 | Saccharin, Na | 90 | 80 | CD |
| 33.21 | Acesulfame K | 80 | 80 | DND |
| 33.22 | Tyramine HCl | 60 | 80 | CD |
| 33.23 | Na Aminobenzoate | 46 | 80 | DND |
| 33.24 | Saccharin, Na | 45 | 80 | DND |
| 33.25 | Tyramine HCl | 30 | 80 | DND |

CD = completely dissolved;
MD = mostly dissolved;
DND = did not dissolve

### Example 33: Profile of HUMIRA®

HUMIRA® (AbbVie Inc., Chicago, Ill.) is a commercially available formulation of the therapeutic monoclonal antibody adalimumab, a TNF-alpha blocker typically prescribed to reduce inflammatory responses of autoimmune diseases such as rheumatoid arthritis, psoriatic arthritis, ankylosing spondylitis, Crohn's disease, ulcerative colitis, moderate to severe chronic psoriasis and juvenile idiopathic arthritis. HUMIRA® is sold in 0.8 mL single use doses containing 40 mg of adalimumab, 4.93 mg sodium chloride, 0.69 mg sodium phosphate monobasic dihydrate, 1.22 mg sodium phosphate dibasic dihydrate, 0.24 mg sodium citrate, 1.04 mg citric acid monohydrate, 9.6 mg mannitol and 0.8 mg polysorbate 80. A viscosity vs. concentration profile of this formulation was generated in the following way. An Amicon Ultra 15 centrifugal concentrator with a 30 kDa molecular weight cut-off (EMD-Millipore, Billerica, Mass.) was filled with about 15 mL of deionized water and centrifuged in a Sorvall Legend RT (ThermoFisher Scientific) at 4000 rpm for 10 minutes to rinse the membrane. Afterwards the residual water was removed and 2.4 mL of HUMIRA® liquid formulation was added to the concentrator tube and was centrifuged at 4000 rpm for 60 minutes at 25° C. Concentration of the retentate was determined by diluting 10 μL of retentate with 1990 μL of deionized water, measuring absorbance of the diluted sample at 280 nm, and calculating the concentration using the dilution factor and extinction coefficient of 1.39 mL/mg-cm. Viscosity of the concentrated sample was measured with a microVisc viscometer equipped with an A05 chip (RheoSense, San Ramon, Calif.) at a shear rate of 250 s⁻¹ at 23° C. After viscosity measurement, the sample was diluted with a small amount of filtrate and concentration and viscosity measurements were repeated. This process was used to generate viscosity values at varying adalimumab concentrations, as set forth in Table 34 below.

TABLE 34

| Adalimumab concentration | |
|---|---|
| (mg/mL) | Viscosity (cP) |
| 277 | 125 |
| 253 | 63 |
| 223 | 34 |
| 202 | 20 |
| 182 | 13 |

### Example 34: Reformulation of HUMIRA® with Viscosity-Reducing Excipient

The following example describes a general process by which HUMIRA® was reformulated in buffer with viscosity-reducing excipient. A solution of the viscosity-reducing excipient was prepared in 20 mM histidine by dissolving about 0.15 g histidine and 0.75 g caffeine (Sigma-Aldrich, St. Louis, Mo.) in deionized water. The pH of the resulting solution was adjusted to about 5 with 5 M hydrochloric acid. The solution was then diluted to a final volume of 50 mL in a volumetric flask with deionized water. The resulting buffered viscosity-reducing excipient solution was then used to reformulate HUMIRA® at high mAb concentrations. Next, about 0.8 mL of HUMIRA® was added to a rinsed Amicon Ultra 15 centrifugal concentrator tube with a 30 kDa molecular weight cutoff and centrifuged in a Sorvall Legend RT at 4000 rpm and 25° C. for 8-10 minutes. Afterwards about 14 mL of the buffered viscosity-reducing excipient solution prepared as described above was added to the concentrated HUMIRA® in the centrifugal concentrator. After gentle mixing, the sample was centrifuged at 4000 rpm and 25° C. for about 40-60 minutes. The retentate was a concentrated sample of HUMIRA® reformulated in a buffer with viscosity-reducing excipient. Viscosity and concentration of the sample were measured, and in some cases then diluted with a small amount of filtrate to measure viscosity at a lower concentration. Viscosity measurements were completed with a microVisc viscometer in the same way as with the concentrated HUMIRA® formulation in the previous example. Concentrations were determined with a Bradford assay using a standard curve generated from HUMIRA® stock solution diluted in deionized water. Reformulation of HUMIRA® with the viscosity-reducing excipient gave viscosity reductions of 30% to 60% compared to the viscosity values of HUMIRA® concentrated in the commercial buffer without reformulation, as set forth in Table 35 below.

TABLE 35

| Adalimumab concentration | |
|---|---|
| (mg/mL) | Viscosity (cP) |
| 290 | 61 |
| 273 | 48 |
| 244 | 20 |
| 205 | 14 |

### Example 35: Improved Stability of Adalimumab Solutions with Caffeine as Excipient

The stability of adalimumab solutions with and without caffeine excipient was evaluated after exposing samples to 2 different stress conditions: agitation and freeze-thaw. The adalimumab drug formulation HUMIRA® (AbbVie) was used, having properties described in more detail in Example 33. The HUMIRA® sample was concentrated to 200 mg/mL adalimumab concentration in the original buffer solution as described in Example 38; this concentrated sample is designated “Sample 1.” A second sample was prepared with ˜200 mg/mL of adalimumab and 15 mg/mL of added caffeine as described in Example 40; this concentrated sample with added caffeine is designated “Sample 2.” Both samples were diluted to a final concentration of 1 mg/mL adalimumab with the diluents as follows: Sample 1 diluent is the original buffer solution, and Sample 2 diluent is a 20 mM histidine, 15 mg/mL caffeine, pH=5. Both HUMIRA® dilutions were filtered through a 0.22 μm syringe filter. For every diluted sample, 3 batches of 300 μL each were prepared in a 2 mL Eppendorf tube in a laminar flow hood. The samples were submitted to the following stress conditions: for agitation, samples were placed in an orbital shaker at 300 rpm for 91 hours; for freeze-thaw, samples were cycled 7 times from −17 to 30° C. for an average of 6 hours per condition. Table 36 describes the samples prepared.

TABLE 36

| Sample # | Excipient added | Stress condition |
|---|---|---|
| 1-C | None | None |
| 1-A | None | Agitation |
| 1-FT | None | Freeze-Thaw |
| 2-C | 15 mg/mL caffeine | None |
| 2-A | 15 mg/mL caffeine | Agitation |
| 2-FT | 15 mg/mL caffeine | Freeze-Thaw |

### Example 36: Evaluation of Stability by Dynamic Light Scattering (DLS)

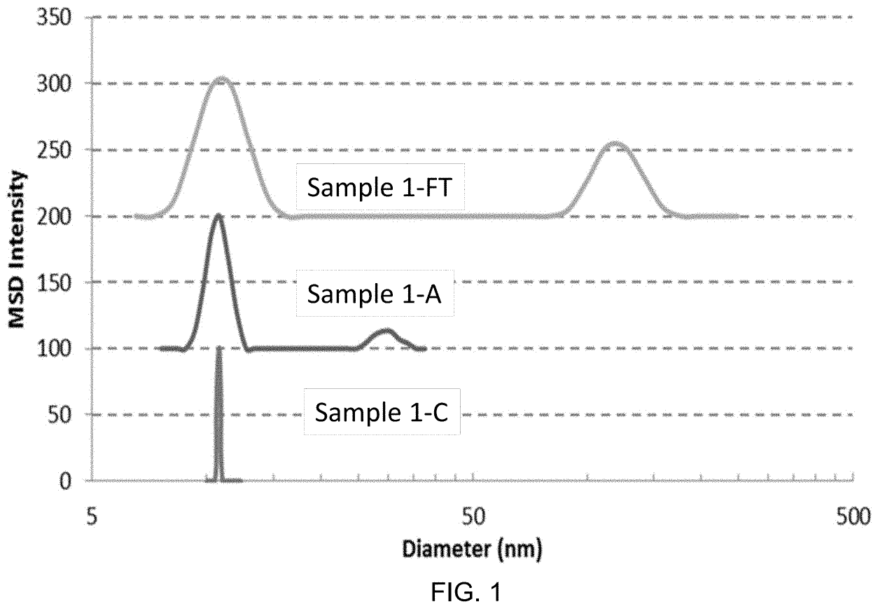

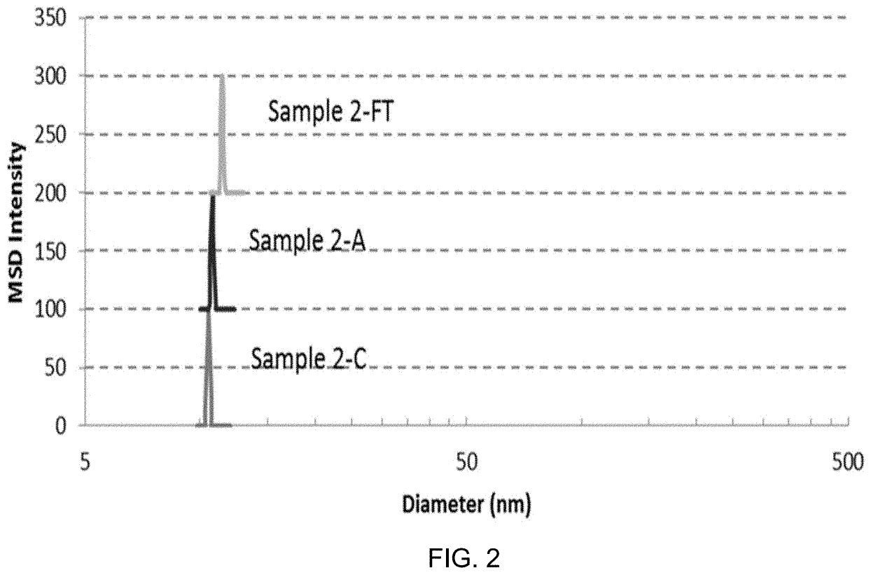

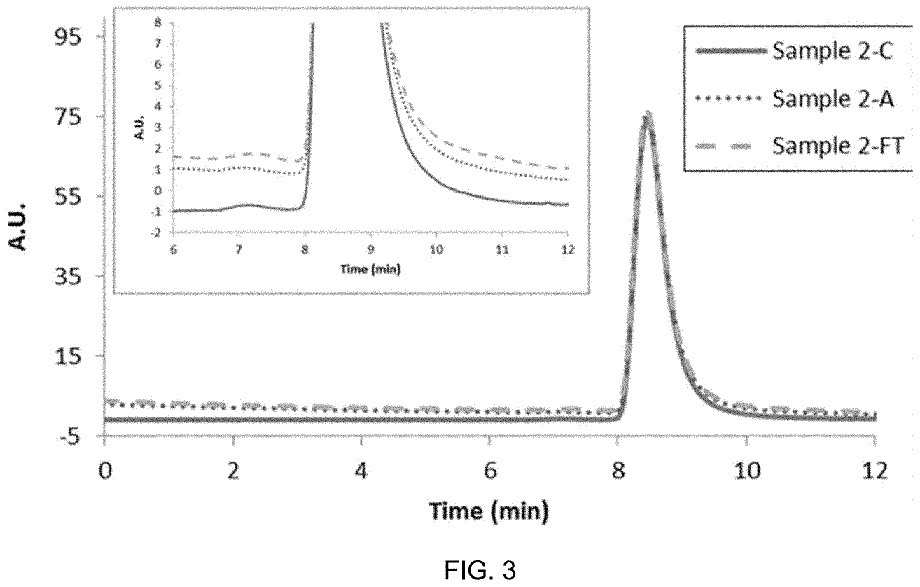

A Brookhaven Zeta Plus dynamic light scattering instrument was used to measure the hydrodynamic radius of the adalimumab molecules in the samples from Example 35, and to look for evidence of the formation of aggregate populations. Table 37 shows the DLS results for the 6 samples prepared according to Example 35: some of them (1-A, 1-FT, 2-A, and 2-FT) had been exposed to stress conditions (“Stressed Samples”), and others (1-C and 2-C) had not been stressed. The DLS data in Table 37, and accompanying FIGS. 1, 2, and 3, show a multimodal particle size distribution of the monoclonal antibody in Stressed Samples that do not contain caffeine. In the absence of caffeine as an excipient, the Stressed Samples 1-A and 1-FT showed higher effective diameter than non-stressed Sample 1-C, and in addition they showed a second population of particles of significantly higher diameter; this new grouping of particles with a larger diameter is evidence of aggregation into subvisible particles. The Stressed Samples containing the caffeine (Samples 2-A and 2-FT) only display one population of particles, at a particle diameter similar to the unstressed Sample 2-C. These results demonstrate that adding caffeine to these samples reduced the formation of aggregates or subvisible particles.

TABLE 37

| | | Diameter | % by | Diameter | % by |
|---|---|---|---|---|---|
| | Effective | of | Intensity of | of | Intensity of |
| | Diameter | Population | Population | Population | Population |
| Sample # | (nm) | #1 (nm) | #1 | #2 (nm) | #2 |
| 1-C | 10.9 | 10.8 | 100 | — | — |
| 1-A | 11.5 | 10.8 | 87 | 28.9 | 13 |
| 1-FT | 20.4 | 11.5 | 66 | 112.2 | 44 |
| 2-C | 10.5 | 10.5 | 100 | — | — |
| 2-A | 10.8 | 10.8 | 100 | — | — |
| 2-FT | 11.4 | 11.4 | 100 | — | — |

Tables 38A and Table 38B display the DLS raw data of adalimumab samples from Example 36 showing the particle size distributions. In these Tables, G(d) is the intensity-weighted differential size distribution. C(d) is the cumulative intensity-weighted differential size distribution.

TABLE 38A

| Sample 1-C | | | Sample 1-A | | | Sample 1-FT | | |
|---|---|---|---|---|---|---|---|---|
| Diameter (nm) | G (d) | C(d) | Diameter (nm) | G (d) | C(d) | Diameter (nm) | G (d) | C(d) |
| 10.6 | 14 | 4 | 9.3 | 13 | 3 | 8.2 | 12 | 2 |
| 10.6 | 53 | 20 | 9.8 | 47 | 15 | 9.2 | 55 | 13 |
| 10.7 | 92 | 46 | 10.3 | 87 | 37 | 10.3 | 98 | 32 |
| 10.8 | 100 | 76 | 10.8 | 100 | 63 | 11.5 | 100 | 52 |
| 10.9 | 61 | 93 | 11.4 | 67 | 80 | 12.9 | 57 | 63 |
| 10.9 | 22 | 100 | 12 | 27 | 87 | 14.5 | 14 | 66 |

| | | | | | |
|---|---|---|---|---|---|
| 26.1 | 4 | 88 | 89.3 | 5 | 67 |
| 27.5 | 10 | 91 | 100.1 | 27 | 72 |
| 28.9 | 13 | 94 | 112.2 | 52 | 83 |
| 30.5 | 13 | 97 | 125.7 | 52 | 93 |
| 32.1 | 7 | 99 | 140.8 | 30 | 99 |
| 33.8 | 4 | 100 | 157.8 | 7 | 100 |

TABLE 38B

| Sample 2-C | | | Sample 2-A | | | Sample 2-FT | | |
|---|---|---|---|---|---|---|---|---|
| Diameter (nm) | G (d) | C(d) | Diameter (nm) | G (d) | C(d) | Diameter (nm) | G (d) | C(d) |
| 10.3 | 14 | 4 | 10.6 | 7 | 2 | 11.3 | 28 | 9 |
| 10.4 | 52 | 19 | 10.6 | 43 | 16 | 11.3 | 64 | 29 |
| 10.5 | 91 | 46 | 10.7 | 79 | 40 | 11.4 | 100 | 60 |
| 10.5 | 100 | 75 | 10.8 | 100 | 71 | 11.5 | 79 | 85 |
| 10.6 | 62 | 93 | 10.8 | 64 | 91 | 11.5 | 43 | 98 |
| 10.7 | 23 | 100 | 10.9 | 29 | 100 | 11.6 | 7 | 100 |

### Example 37: Evaluation of Stability by Size-Exclusion Chromatography (SEC)

Size exclusion chromatography was used to detect subvisible particulates of less than about 0.1 microns in size from the stressed and unstressed adalimumab samples described in Example 36. To perform the SEC, a TSKgel SuperSW3000 column (Tosoh Biosciences, Montgomeryville, Pa.) with a guard column was used, and the elution was monitored at 280 nm. A total of 10 μL of each stressed and unstressed sample from Example 36 was eluted isocratically with a pH 6.2 buffer (100 mM phosphate, 325 mM NaCl), at a flow rate of 0.35 mL/min. The retention time of the adalimumab monomer was approximately 9 minutes. No detectable aggregates were identified in the samples containing the caffeine excipient, and the amount of monomer in all 3 samples remained constant.

### Example 38: Viscosity Reduction of HERCEPTIN® Formulation

The monoclonal antibody trastuzumab (HERCEPTIN® from Genentech) was received as a lyophilized powder and reconstituted to 21 mg/mL in DI water. The resulting solution was concentrated as-is in an Amicon Ultra 4 centrifugal concentrator tube (molecular weight cut-off, 30 kDa) by centrifuging at 3500 rpm for 1.5 hrs. The concentration was measured by diluting the sample 200 times in an appropriate buffer and measuring absorbance at 280 nm using the extinction coefficient of 1.48 mL/mg. Viscosity was measured using a RheoSense microVisc viscometer.

Excipient buffers were prepared containing salicylic acid and caffeine either alone or in combination by dissolving histidine and excipients in distilled water, then adjusting pH to the appropriate level. The conditions of Buffer Systems 1 and 2 are summarized in Table 39.

TABLE 39

| Buffer System | Salicylic Acid | Caffeine | Osmolality | |
|---|---|---|---|---|
| # | concentration | concentration | (mOsm/kg) | pH |
| 1 | 10 mg/mL | 10 mg/mL | 145 | 6 |
| 2 | 0 | 15 mg/mL | 86 | 6 |

HERCEPTIN® solutions were diluted in the excipient buffers at a ratio of ˜1:10 and concentrated in Amicon Ultra 15 (MWCO 30 kDa) concentrator tubes. Concentration was determined using a Bradford assay and compared with a standard calibration curve made from the stock HERCEPTIN® sample. Viscosity was measured using the RheoSense microVisc viscometer. The concentration and viscosity measurements of the various HERCEPTIN® solutions are shown in Table 40 below, where Buffer Systems 1 and 2 refer to those buffers described in Table 39.

TABLE 40

| | | Buffer System 1: | | | |
|---|---|---|---|---|---|
| Control | | Solution with | | Buffer System 2: | |
| solution with | | 10 mg/mL Caffeine + | | Solution with | |
| no added | | 10 mg/mL Salicylic | | 15 mg/mL | |
| excipients | | Acid added | | Caffeine added | |
| | Antibody | | Antibody | | Antibody |
| Vis- | Concen- | Vis- | Concen- | Vis- | Concen- |
| cosity | tration | cosity | tration | cosity | tration |
| (cP) | (mg/mL) | (cP) | (mg/mL) | (cP) | (mg/mL) |
| 37.2 | 215 | 9.7 | 244 | 23.4 | 236 |
| 9.3 | 161 | 7.7 | 167 | 12.2 | 200 |
| 3.1 | 108 | 2.9 | 122 | 5.1 | 134 |
| 1.6 | 54 | 2.4 | 77 | 2.1 | 101 |

Buffer System 1, containing both salicylic acid and caffeine, had a maximum viscosity reduction of 76% at 215 mg/mL compared to the control sample. Buffer System 2, containing just caffeine, had viscosity reduction up to 59% at 200 mg/mL.

### Example 39: Viscosity Reduction of AVASTIN® Formulation

AVASTIN® (monoclonal antibody bevacizumab formulation marketed by Genentech) was received as a 25 mg/mL solution in a histidine buffer. The sample was concentrated in Amicon Ultra 4 centrifugal concentrator tubes (MWCO 30 kDa) at 3500 rpm. Viscosity was measured by RheoSense microVisc and concentration was determined by absorbance at 280 nm (extinction coefficient, 1.605 mL/mg). The excipient buffer was prepared by adding 10 mg/mL caffeine along with 25 mM histidine HCl. AVASTIN® stock solution was diluted with the excipient buffer then concentrated in Amicon Ultra 15 centrifugal concentrator tubes (MWCO 30 kDa). The concentration of the excipient samples was determined by Bradford assay and the viscosity was measured using the RheoSense microVisc. Results are shown in Table 41 below.

TABLE 41

| | | Viscosity | |
|---|---|---|---|
| | Viscosity | with 10 mg/mL | |
| | without added | added caffeine | % Viscosity |
| Concentration | excipient | excipient | Reduction from |
| (mg/mL) | (cP) | (cP) | Excipient |
| 266 | 297 | 113 | 62% |
| 213 | 80 | 22 | 73% |
| 190 | 21 | 13 | 36% |

AVASTIN® showed a maximum viscosity reduction of 73% when concentrated with 10 mg/mL of caffeine to 213 mg/mL when compared to the control AVASTIN® sample.

### Example 40: Preparation of Formulations Containing Caffeine, a Secondary Excipient and Test Protein

Formulations were prepared using caffeine as the excipient compound or a combination of caffeine and a second excipient compound, and a test protein, where the test protein was intended to simulate a therapeutic protein that would be used in a therapeutic formulation. Such formulations were prepared in 20 mM histidine buffer with different excipient compounds for viscosity measurement in the following way. Excipient combinations (Excipients A and B, as described in Table 42 below) were dissolved in 20 mM histidine and the resulting solution pH adjusted with small amounts of sodium hydroxide or hydrochloric acid to achieve pH 6 prior to dissolution of the model protein. Once excipient solutions had been prepared, the test protein bovine gamma globulin (BGG) was dissolved at a ratio to achieve a final protein concentration of about 280 mg/mL. Solutions of BGG in the excipient solutions were formulated in 20 mL glass scintillation vials and allowed to shake at 80-100 rpm on an orbital shaker table overnight. BGG solutions were then transferred to 2 mL microcentrifuge tubes and centrifuged for about ten minutes at 2300 rpm in an IEC MicroMax microcentrifuge to remove entrained air prior to viscosity measurement.

Viscosity measurements of formulations prepared as described above were made with a DV-IIT LV cone and plate viscometer (Brookfield Engineering, Middleboro, Mass.). The viscometer was equipped with a CP-40 cone and was operated at 3 rpm and 25° C. The formulation was loaded into the viscometer at a volume of 0.5 mL and allowed to incubate at the given shear rate and temperature for 3 minutes, followed by a measurement collection period of twenty seconds. This was then followed by 2 additional steps consisting of 1 minute of shear incubation and subsequent twenty second measurement collection period. The three data points collected were then averaged and recorded as the viscosity for the sample in Table 42 below. Viscosities of solutions with excipient were normalized to the viscosity of the model protein solution without excipient. The normalized viscosity is the ratio of the viscosity of the model protein solution with excipient to the viscosity of the model protein solution with no excipient.

TABLE 42

| Excipient A | | Excipient B | | |
|---|---|---|---|---|
| | Conc. | | Conc. | Normalized |
| Name | (mg/mL) | Name | (mg/mL) | Viscosity |
| — | 0 | — | 0 | 1.00 |
| Caffeine | 15 | — | 0 | 0.77 |
| Caffeine | 15 | Sodium acetate | 12 | 0.77 |
| Caffeine | 15 | Sodium sulfate | 14 | 0.78 |
| Caffeine | 15 | Aspartic acid | 20 | 0.73 |
| Caffeine | 15 | CaCl₂ dihydrate | 15 | 0.65 |
| Caffeine | 15 | Dimethyl Sulfone | 25 | 0.65 |
| Caffeine | 15 | Arginine | 20 | 0.63 |
| Caffeine | 15 | Leucine | 20 | 0.69 |
| Caffeine | 15 | Phenylalanine | 20 | 0.60 |
| Caffeine | 15 | Niacinamide | 15 | 0.63 |
| Caffeine | 15 | Ethanol | 22 | 0.65 |

### Example 41: Preparation of Formulations Containing Dimethyl Sulfone and Test Protein

Formulations were prepared using dimethyl sulfone (Jarrow Formulas, Los Angeles, Calif.) as the excipient compound and a test protein, where the test protein was intended to simulate a therapeutic protein that would be used in a therapeutic formulation. Such formulations were prepared in 20 mM histidine buffer for viscosity measurement in the following way. Dimethyl sulfone was dissolved in 20 mM histidine and the resulting solution pH adjusted with small amounts of sodium hydroxide or hydrochloric acid to achieve pH 6 and then filtered through a 0.22 micron filter prior to dissolution of the model protein. Once excipient solutions had been prepared, the test protein bovine gamma globulin (BGG) was dissolved at a concentration of about 280 mg/mL. Solutions of BGG in the excipient solutions were formulated in 20 mL glass scintillation vials and allowed to shake at 80-100 rpm on an orbital shaker table overnight. BGG solutions were then transferred to 2 mL microcentrifuge tubes and centrifuged for about ten minutes at 2300 rpm in an IEC MicroMax microcentrifuge to remove entrained air prior to viscosity measurement.

Viscosity measurements of formulations prepared as described above were made with a DV-IIT LV cone and plate viscometer (Brookfield Engineering, Middleboro, Mass.). The viscometer was equipped with a CP-40 cone and was operated at 3 rpm and 25° C. The formulation was loaded into the viscometer at a volume of 0.5 mL and allowed to incubate at the given shear rate and temperature for 3 minutes, followed by a measurement collection period of twenty seconds. This was then followed by 2 additional steps consisting of 1 minute of shear incubation and subsequent twenty second measurement collection period. The three data points collected were then averaged and recorded as the viscosity for the sample. Viscosities of solutions with excipient were normalized to the viscosity of the model protein solution without excipient. The normalized viscosity recorded in Table 43 is the ratio of the viscosity of the model protein solution with excipient to the viscosity of the model protein solution with no excipient.

TABLE 43

| Dimethyl sulfone concentration (mg/mL) | Normalized viscosity |
|---|---|
| 0 | 1.00 |
| 15 | 0.92 |
| 30 | 0.71 |
| 50 | 0.71 |
| 30 | 0.72 |

### Example 42: Preparation of Buffer Solutions

A buffer of 20 mM 2-(N-morpholino) ethanesulfonic acid (MES), 50 mM glycine and 35 mM caffeine was prepared by dissolving 0.392 g MES monohydrate, 0.374 g glycine and 0.682 g caffeine in 90 mL of Milli-Q ultrapure water. After all contents were dissolved, the solution pH was adjusted to 5.5 and final volume of 100 mL by adding Milli-Q ultrapure water in volumetric flask. The buffer solution was then vacuum filtered through a 0.2 μm PES filter using a bottle top filter device. A similar buffer containing 20 mM histidine, 50 mM glycine and 35 mM caffeine was also prepared in the same way.

A control buffer of 20 mM tris(hydroxymethyl)aminomethane (TRIS), 100 mM sodium chloride, 55 mM mannitol and 0.1 mM diethylenetriaminepentaacetic acid (DTPA) was prepared by dissolving 1.211 g TRIS, 2.938 g sodium chloride, 2.098 g mannitol and 0.019 g DTPA in 450 mL of Milli-Q ultrapure water. After all contents were dissolved, the solution pH was adjusted to 7.0 and the volume was adjusted to 500 mL by adding Milli-Q ultrapure water in a volumetric flask. The buffer solution was vacuum filtered through a 0.2 μm PES filter using a bottle top filter device.

### Example 43: Ipilimumab Formulations

A sample of the monoclonal antibody ipilimumab was acquired from Bioceros (The Netherlands) and buffer exchanged into the three prepared buffers of Example 42 using Amicon Ultra 15 centrifugal concentrator tubes with a 30 kDa molecular weight cut-off (EMD Millipore, Billerica, Mass.). The target final protein concentration was 20 mg/mL, and the final concentration was measured by absorbance at 280 nm (A280) with Synergy HT plate reader (BioTek, Winooski, Vt.). The absorbance of protein solution was subtracted from the absorbance of a blank buffer solution. The blank-subtracted protein solution absorbance is divided by the reported extinction coefficient and then multiplied by the protein dilution factor (20×) to determine the final protein concentration. Since caffeine interferes with absorbance measurement at 280 nm, the protein concentration of solutions containing caffeine were determined by mass balance against A280-measured protein solution. This gave an approximate concentration close to the measured A280 protein solution based on mass.

The prepared protein solutions were then added to a 384 micro-well plate (Aurora Microplates, Whitefish, Mont.). Each solution was loaded into three wells at 35 μL per well. The micro-well plate was then centrifuged at 400×g in a Sorvall Legend RT centrifuge to remove any encapsulated air pockets. A pre-cut, pressure sensitive sealing tape (Thermo Scientific) was applied on top of the micro-well plate to prevent evaporation before placing into DLS instrument (DynaPro II DLS plate reader, Wyatt Technology Corp., Goleta, Calif.). The DLS instrument sample compartment was held at 65° C. and particle size of protein solutions was recorded for 9 hours. Table 44 shows radius size for ipilimumab in three different formulations at 1-hour measurements.

TABLE 44

| | DLS particle size (radius in nm) | | |
|---|---|---|---|
| | of Ipilimumab @ 65° C. | | |
| | Buffer system: 20 mM | Buffer system: 20 mM | |
| | MES, 50 mM glycine, | histidine, 50 mM glycine, | |
| Time | 35 mM caffeine, | 35 mM caffeine, | |
| (h) | pH 6.0 | pH 6.0 | Control |
| 0 | 40.8 | 32.1 | 11.5 |
| 1 | 14.4 | 12.7 | 8.9 |
| 2 | 13.0 | 12.3 | 10.4 |
| 3 | 11.3 | 11.3 | 12.4 |
| 4 | 10.9 | 11.4 | 15.1 |
| 5 | 10.1 | 11.4 | 18.8 |
| 6 | 9.6 | 11.8 | 23.5 |
| 7 | 9.4 | 12.2 | 29.9 |
| 8 | 9.5 | 12.8 | 39.0 |
| 9 | 9.5 | 13.0 | 55.5 |

### Example 44: Testing Stability of Protein Formulations by Urea Denaturation

It is known that urea denatures proteins in solution and causes them to unfold. The screening methodology in this example involved adding a specific concentration of urea to a therapeutic protein solution such as ustekinumab. This test example was based on the hypothesis that protective excipients would prevent or diminish the unfolding of a therapeutic protein in the presence of urea, and measuring the amount of unfolded protein would allow one to identify the excipients that were effective at stabilizing the protein in the presence of urea. One method to track protein unfolding involves the use of extrinsic fluorescent dyes such as Sypro orange. Sypro orange binds to hydrophobic regions in the unfolded protein structure, leading to an increase in the fluorescence signal observed. Measuring the differences in the fluorescence intensity of unfolded protein-Sypro orange complex in presence of different excipients thus allows one to identify any stabilizing effects.

All excipients used in this Example, listed in Table 45 below, were of the highest purity, and were obtained from Sigma Aldrich (St. Louis, Mo.) or Cayman Chemical (Ann Arbor, Mich.). Stock solutions of the excipients were prepared by dissolving each of the excipients at a concentration of 100 mg/mL in 20 mM histidine buffer, pH 6.0. The histidine buffer had been prepared by dissolving 1.55 g of histidine in 0.500 L Milli-Q water and adjusting the pH to 6.0 using 1 M HCl. Then, an excipient-containing protein formulation was prepared by combining each excipient preparation to a final concentration of 5 mg/mL with ustekinumab to a final concentration of 1 mg/mL. A stock solution of 9M urea had been prepared for use in this Example by dissolving 27 g of urea in the same histidine buffer and the pH adjusted to 6.0 using 1 M HCl. This urea stock solution was then added to a final concentration of 6M to produce the test solutions (excipient plus protein plus urea). The Sypro orange dye from the stock solution (5000×) was then spiked into a final concentration of 20× for each. The pH of the mixture was rechecked and confirmed to be at pH 6.0. The test solutions were allowed to incubate at room temperature for 30 min. 200 μL of the samples were transferred to a Greiner CellStar black well clear flat-bottom 96 well plate and the fluorescence of each sample was measured using a BioTek Synergy HT plate reader with an excitation of 485 nm and emission filters of 590/20 nm. The fluorescence intensities of the different test formulations were compared to that of the control formulation (protein and urea without any excipient) and those test formulations exhibiting decreased fluorescence were considered to include stabilizing excipients. As shown in Table 45 below, a number of excipients reflected increased stability as compared to the control, when their fluorescence was compared to that of the control. These conclusions were drawn because an excipient's ability to stabilize the protein correlates with its ability to decrease the fluorescence measured during the experiment: a stabilizing excipient would prevent or reduce protein unfolding, which would lead to a decrease in the protein-Sypro orange interactions, which in turn would be manifested as a diminished fluorescent intensity. The results of these tests are summarized in Table 45 below.

TABLE 45

| Test No. | Excipient | % increased stability |
|---|---|---|
| 1 | Castanospermine | 7.0 |
| 2 | Theanine | 6.1 |
| 3 | 4-phenylbutyric acid | 9.0 |
| 4 | p-aminobenzoic acid | 2.7 |
| 5 | Arabitol | 2.7 |
| 6 | Sedoheptulose | 3.7 |
| 7 | Nicotinamide | 2.7 |
| 8 | Xylitol | 6.3 |
| 9 | isonicotinic acid | 4.4 |
| 10 | Spermine | 16.8 |
| 11 | Spermidine | 12.9 |
| 12 | Cystamine | 8.8 |
| 13 | Neamine | 2.8 |
| 14 | Tryptamine | 11.6 |
| 15 | Cytidine | 1.6 |
| 16 | methyl cytidine | 4.0 |
| 17 | benzamide oxime | 1.4 |
| 18 | Nicotinamide adenine dinucleotide | 32.5 |
| 19 | Adenosine | 53.6 |
| 20 | Melzitose | 4.7 |
| 21 | Raffinose | 1.7 |

### Example 45: Stabilization of Protein Formulations at Low pH

Therapeutic proteins, particularly antibodies are exposed to low pH solution conditions during different stages of processing, especially purification and viral clearance. This exposure to acidic pH conditions can lead to conformational changes, which in turn lead to unfolding and aggregation of the protein. The screening methodology to identify stabilizing excipients involved incubating a therapeutic protein such as omalizumab at an acidic pH. Protective excipients would prevent or diminish the unfolding of a therapeutic protein at low pH, so measuring the amount of unfolded protein would allow one to identify the excipients that were effective at stabilizing the protein in the presence of low pH. The unfolding of therapeutic proteins at acidic pH can be followed using extrinsic fluorescent dyes such as Sypro orange. Addition of Sypro orange (Thermo-Fisher, Waltham Mass.), as performed in this Example, allows the dye to bind to hydrophobic regions in the unfolded protein, leading to an increase in the fluorescence signal. Measuring the differences in the fluorescence intensities of the unfolded protein-Sypro orange in presence of difference excipients thus allows one to identify stabilizers.

All excipients used in this Example, listed in Table 46 below, were of the highest purity, and were obtained from Sigma Aldrich (St. Louis, Mo.) or Cayman Chemical (Ann Arbor, Mich.). Stock solutions of the excipients were prepared by dissolving each of the excipients at a concentration of 100 mg/mL in 0.15 M glycine buffer pH 2.6. The acidification buffer was prepared by dissolving 1.65 g of histidine in 0.09 L Milli-Q water, adjusting the pH to 2.6 using 1 M HCl, and making the volume to 0.100 L. Then, each excipient-containing protein formulation was prepared by combining each excipient preparation to a final concentration of 5 mg/mL with ustekinumab to final concentration of 1 mg/mL. The glycine acidification buffer was then added to the excipient-protein mixture followed by spiking in the Sypro orange dye from the stock solution (5000×) to a final concentration of 20×. 200 μL of the samples were transferred to a Greiner CellStar black well clear flat-bottom 96 well plate and fluorescence of each sample was measured using a BioTek Synergy HT plate reader with an excitation wavelength of 485 nm and emission filter wavelength of 590/20 nm. The fluorescence intensities of the different test formulations were compared to that of the control formulation (protein and glycine buffer without any excipient) and those exhibiting decreased fluorescence were considered to include stabilizing excipients. As shown in Table 46, a number of excipients reflected increased stability as compared to the control, when their fluorescence was compared to that of the control. These conclusions were drawn because an excipient's ability to stabilize the protein correlates with its ability to decrease the fluorescence measured during the experiment: a stabilizing excipient would prevent or reduce protein unfolding, which would lead to a decrease in the protein-Sypro orange interactions, which in turn would be manifested as a diminished fluorescent intensity. The results of these tests are summarized in Table 46 below.

TABLE 46

| Test No. | Excipient | % change in stability |
|---|---|---|
| 1 | Cytidine | 79.8 |
| 2 | Melzitose | 2.0 |
| 3 | Arabitol | 3.4 |
| 4 | Erythritol | 1.8 |
| 5 | Cytidine monophosphate | 83.1 |
| 6 | Iditol | 11.6 |
| 7 | Xylitol | 0.4 |
| 8 | Lactitol | 0.6 |
| 9 | Psicose | 1.1 |
| 10 | Sedoheptulose | 2.6 |
| 11 | Emtricitabine | 2.9 |
| 12 | Methyl cytidine | 21.7 |
| 13 | Raffinose | 2.6 |
| 14 | Cystamine | 17.8 |
| 15 | Spermine | 90.0 |
| 16 | Mannose | 9.4 |
| 17 | Trehalose | 2.5 |
| 18 | Pullulan | 20.0 |
| 19 | Aminobenzoic acid | 43.0 |
| 20 | Allyl cysteine | 26.9 |
| 21 | Neamine | 5.9 |
| 22 | Benzamide oxime | 70.6 |
| 23 | Isonicotinamide | 65.4 |
| 24 | Diethylenetriaminepentaacetic acid | 11.8 |
| 25 | Meglumine | 41.8 |
| 26 | Pyridyl ethylamine | 85.6 |
| 27 | Spermidine | 98.1 |
| 28 | Theanine | 8.3 |
| 29 | Castanospermine | 95.9 |
| 30 | Adenosine | 32.2 |

### Example 46: Tests of Excipients as Thermal Stabilizers

Therapeutic proteins are frequently subjected to fluctuations in temperatures which may lead to changes in tertiary and secondary structural elements. This can lead to aggregation of the protein and decrease the amount of active native protein. Excipients protecting against thermal stress were identified in this Example by thermal degradation studies in the presence or absence of the excipients set forth in Table 47 below. All excipients used in this Example, listed in Table 47 below, were of the highest purity, and were obtained from Sigma Aldrich (St. Louis, Mo.) or Cayman Chemical (Ann Arbor, Mich.). Stock solutions of the excipients were prepared by dissolving each of the excipients at a concentration of 100 mg/mL in 20 mM histidine buffer at pH 6.0. The histidine buffer had been prepared by dissolving 1.55 g of histidine in 0.500 L Milli-Q water and adjusting the pH to 6.0 using 1 M HCl. Then, an excipient-containing protein formulation was prepared by combining each excipient preparation to a final concentration of 5 mg/mL with ustekinumab to final concentration of 1 mg/mL. The formulation was aliquoted into 0.2 mL microcentrifuge tubes and incubated at 65° C. in a heating block for 120 min. The aliquots were withdrawn at 0, 15, 30, 60, 90 and 120 minutes. The samples were quenched on ice for 5 minutes and spun down at 5000 rpm for 10 minutes. The samples were then analyzed by size exclusion-HPLC where the supernatant was loaded onto an Agilent 1100 HPLC system fitted with TSKgel SW3000 column (30 cm×4.8 mm ID) and Agilent G1351B diode array detector set to 280 nm. The mobile phase of 50 mM phosphate buffer, 100 mM NaCl at pH 6.5 was used at a flow rate of 0.35 mL/min. The monomer fraction was calculated by integrating the monomer peak area and changes in the integrated peak area was plotted as a function of time. The thermal stability was correlated with the fraction of monomer remaining at the end of the 2 h incubation.

TABLE 47

| | Monomer Fraction % |
|---|---|
| Excipient added | change from control |
| Sedoheptulose | 3.4 |
| Melzitose | 2.0 |
| Arabitol | 6.0 |
| Pullulan | 1.5 |
| Adenosine | 8.4 |
| Nicotinamide adenine dinucleotide | 9.1 |

### Example 47: Tests of Excipients as Stabilizers Against Mechanical Shear Stresses

Therapeutic proteins often subjected to mechanical stress by agitation, stirring etc., and the imparted shear stress can lead to aggregation of the protein. Excipients that offer protection against shear stresses were identified by agitating therapeutic protein solutions in the presence of the test excipients (listed in Table 48 below) and observing any change in the number of aggregated particles. All excipients used in this Example, listed in Table 48 below, were of the highest purity, and were obtained from Sigma Aldrich (St. Louis, Mo.) or Cayman Chemical (Ann Arbor, Mich.). Stock solutions of the excipients were prepared by dissolving each of the excipients at a concentration of 100 mg/mL in Milli-Q water. Then, an excipient-containing protein formulation was prepared by combining each excipient preparation to a final concentration of 0.5 mg/mL with omalizumab to final concentration of 2 mg/mL. The samples were transferred to a 0.5 mL cryogenic vial (ThermoFisher, Waltham Mass.) and secured to an orbital shaker. They were then incubated at 22° C. for 72 h with agitation set at 300 rpm. At the end of incubation period, 100 μL of each sample was transferred to 96-well plates, and the absorbance was measured at 350 nm. Any increase in aggregation was measured by observing the changes in light scattering at 350 nm, and comparing those changes to the light scattering at 350 nm of a control formulation (prepared identically to the test samples but without the addition of the excipient). Excipients that are protective in nature were identified by their ability to slow the rate of aggregation and decrease the A350 absorbance value after the agitation step.

TABLE 48

| Excipient | % decrease of A350 signal vs. control |
|---|---|
| Rebaudioside A | 73.2 |
| Madecassoside | 73.2 |
| Tubeimoside | 74.5 |
| Mogroside V | 68.2 |
| Harpagoside | 69.0 |
| Hederacoside | 58.9 |

### Example 48: Freeze-Thaw Stability of Protein Solutions with Excipients

All excipients used in this Example, listed in Table 49 below, were obtained from Sigma Aldrich (St. Louis, Mo.) or Cayman Chemical (Ann Arbor, Mich.). Stock solutions of test excipients (as listed in Table 49) were prepared by dissolving the each of the excipients at a concentration of 100 mg/mL in 20 mM histidine buffer at pH 6.0. The buffer was prepared by dissolving 1.55 g of histidine in 0.500 L Milli-Q water and adjusting the pH to 6.0 using 1 M HCl. Then, an excipient-containing protein formulation was prepared by combining each excipient preparation to a final concentration of 5 mg/mL with omalizumab to final concentration of 5 mg/mL. 0.4 mL of each formulation was transferred to the wells within a 96-well polypropylene plate (Advangene, IL). The samples were frozen to −80° C. in a freezer and then thawed at room temperature for at least 5 cycles, then 100 μL of the samples were transferred to a Greiner CellStar black well clear flat-bottom 96-well plate. The stabilizing effect of the excipients was analyzed by measuring the formation of protein aggregates using light scattering analysis at 350 nm, and comparing those changes to the light scattering at 350 nm of a control formulation (prepared identically to the test samples but without the addition of the excipient). Excipients that are protective in nature were identified by their ability to slow the rate of aggregation and decrease the A350 absorbance value after the agitation step.

TABLE 49

| Excipient | % reduction in light scattering at 350 nm vs. control |
|---|---|
| Arabinogalactan | 15.3 |
| Raffinose | 3.7 |
| Melezitose | 7.4 |
| Pullulan | 4.0 |
| seduheptalose | 16.0 |
| Arabitol | 17.8 |
| Iditol | 11.7 |
| Psicose | 15.3 |
| Meglumine | 15.3 |
| DTPA | 17.2 |
| allyl cysteine | 4.9 |
| isonicotinamide | 4.8 |
| Xylitol | 10.7 |
| Mannitol | 17.8 |

### Example 49: Excipient Testing by DLS Diffusion Interaction Parameter kD

A stock solution of 20 mM histidine hydrochloride (His HCl) buffer was prepared for use in formulating excipient and protein solutions by dissolving 3.1 g of histidine (Sigma-Aldrich, St. Louis, Mo.) in Type 1 ultrapure water. The resulting solution was titrated to pH 6 by dropwise addition of 1 M hydrochloric acid. After pH adjustment, the buffer was diluted to a final volume of 1 L in a volumetric flask with Type 1 ultrapure water. All excipients used in this Example, listed in Table 50 below, were of the highest purity, and were obtained from Sigma Aldrich (St. Louis, Mo.) or Cayman Chemical (Ann Arbor, Mich.).

A series of six test protein solutions were prepared using the proteins described in Table 50, ranging in protein concentration from about 4 mg/mL to about 20 mg/mL, all in 20 mM His HCl buffer at pH 6. In a 384-well microplate (Aurora Microplates, Whitefish, Mont.), 15 L of protein solution was combined with 15 μL of a stock excipient solution prepared in 20 mM His HCl buffer at pH 6, using the excipients described in Table 50, such that each excipient was tested at 6 different protein concentrations. The microplate containing the protein-excipient combinations was centrifuged at 400×g in a Sorvall Legend RT centrifuge and then shaken on a plate shaker to adequately mix the samples. A second centrifuge step was completed to remove air bubbles. The diffusion interaction parameter (kD) of these protein-excipient formulations was measured by dynamic light scattering (DLS) in dilute solution as a way of probing the impact of excipients on protein-protein interactions (PPI). To perform the DLS studies, the microplate prepared above was loaded into a DynaPro II DLS plate reader (Wyatt Technologies Corp., Goleta, Calif.) and the diffusion coefficient of each sample was measured at 25° C. For each excipient-containing test solution, the measured diffusion coefficient was plotted as a function of protein concentration, and the slope of the linear fit of the data was recorded as the kD. A more negative kD indicated a stronger net attractive PPI and a more positive kD indicated a stronger net repulsive PPI. Table 50 sets forth the kD values of each excipient-containing test solution, where these test values for each excipient can be compared to the kD value of the control solution (containing protein in the histidine buffer but no excipient).

TABLE 50

| | Excipient | | |
|---|---|---|---|
| | stock con- | Omalizumab | Ustekinumab |
| | centration | k_{D} | k_{D} |
| Excipient ID | (mM) | (mL/g) | (mL/g) |
| Control (none) | 0 | −24.6 | 28.3 |
| Isoguvacine HCl | 200 | −7.9 | 1.0 |
| Cycloserine | 200 | −14.7 | −13.6 |
| 4-Aminobenzoic acid | 200 | −17.4 | 1.5 |
| DL-Norepinephrine HCl | 100 | −19.8 | 1.5 |
| Homovanillic acid | 200 | −21.2 | 5.5 |
| 1-methyl-4-imidazoleacetic | 200 | −21.8 | 47.4 |
| acid | | | |
| 18-crown-6 ether | 200 | −22.2 | 32.3 |
| Piracetam | 200 | −23.6 | 27.3 |
| 1-aminobenzotriazole | 200 | −24.1 | 24.0 |
| rasagiline mesylate | 100 | −24.1 | 26.5 |
| 2-Methylimidazole | 200 | −24.5 | −0.5 |
| 15-crown-5 ether | 200 | −25.0 | 26.7 |
| Chloroquine Phosphate | 100 | −26.4 | 15.2 |
| 4-hydroxy-3-methoxycinnamic | 200 | −26.6 | 21.0 |
| acid | | | |
| Benzyl acetonacetate | 100 | −27.0 | 26.6 |
| Guanfacine HCl | 100 | — | 36.1 |
| Kojic Acid | 200 | −29.1 | 33.2 |
| 3-(1-Pyridinio)-1- | 200 | −30.2 | 26.7 |
| propanesulfonate | | | |
| pyridoxine HCl | 200 | −4.3 | −0.5 |
| Tetramethylethylenediamine | 200 | −14.6 | 0.1 |
| HCl | | | |
| L-ornithine | 200 | −16.5 | −4.3 |
| Sodium borate | 200 | −28.8 | 17.9 |

### Example 50: Excipient Testing for Viscosity Reduction

Biosimilar monoclonal antibodies omalizumab and ustekinumab, acquired from Bioceros (The Netherlands), were buffer-exchanged into 20 mM His HCl buffer at pH 6 and concentrated using Amicon Ultra 15 centrifugal concentrator tubes with a 30 kDa molecular weight cut-off (EMD Millipore, Billerica, Mass.). The resulting concentrated formulations were analyzed by absorbance at 280 nm for protein concentration by making serial dilutions of the concentrated formulation in 20 mM His HCl, loading 100 μL of each dilution into a UV clear 96 half-well microplate (Greiner Bio-One, Austria), and measuring absorbance at 280 nm with a Synergy HT plate reader (BioTek, Winooski, Vt.). The blanked, path-length corrected absorbance measurement was then divided by the respective extinction coefficient and multiplied by the dilution factor to determine the protein concentration. Excipient solutions were prepared in 20 mM HisHCl pH 6 at 10× the desired final concentration or the solubility limit of the compound, and pH adjusted to 6 as necessary with either concentrated hydrochloric acid or sodium hydroxide. Concentrated protein formulation was then combined with a 10× excipient solution of the excipients listed in Table 51 below (9 parts protein, 1 part excipient solution or buffer) in a 384-well microplate (Aurora Microplates, Whitefish, Mont.). All excipients used in this Example, listed in Table 51 below, were of the highest purity, and were obtained from Sigma Aldrich (St. Louis, Mo.) or Cayman Chemical (Ann Arbor, Mich.). The concentration of protein in each sample is the same since each sample was diluted by the same volume. The microplate was then centrifuged at 400×g in a Sorvall Legend RT centrifuge and shaken on a plate shaker. After shaking, 2 μL of a 5-fold dilution of polyethylene glycol surface-modified gold nanoparticles (nanoComposix, San Diego, Calif.) in 20 mM His HCl was added to each sample well. The microplate was shaken a second time to mix the gold nanoparticles into the sample, and then placed in a DynaPro II DLS plate reader (Wyatt Technology Corp., Goleta, Calif.) to measure the apparent particle size of the gold nanoparticles at 25° C. The ratio of the apparent particle size of the gold nanoparticle in a protein formulation to the known particle size of the gold nanoparticle in water was used to determine the viscosity of the protein formulation according to the Stokes-Einstein equation. In this Example, the ratio of apparent radius to the actual radius of the gold nanoparticles was multiplied by the viscosity of water at 25° C. to calculate the viscosity of the protein formulation in centipoise (cP). The results of these tests are summarized in Table 51 below.

TABLE 51

| Excipient Solution | | | | |
|---|---|---|---|---|
| | Excipient | Final | Omalizumab | Ustekinumab |
| | mass | volume | viscosity | viscosity |
| Excipient name | (g) | (mL) | (cP) | (cP) |
| 3-Aminobenzamide | 0.023 | 0.2 | 105 | 12.8 |
| N,N-Dimethyl- | 0.435 | 5 | 83 | 12.6 |
| acetamide | | | | |
| 3-(1-Pyridinio)- | 1.036 | 5 | 82 | — |
| 1-propanesulfonate | | | | |
| Sulfolane | 0.593 | 5 | 76 | 10.6 |
| 1,3-Dimethyl- | 0.643 | 5 | 72 | 11.4 |
| 3,4,5,6-tetra- | | | | |
| hydro-2-(1H)- | | | | |
| pyrimidone | | | | |
| Acetoin | 0.439 | 5 | 71 | 8.7 |
| diaminopimelic | 0.388 | 2 | 68 | — |
| acid | | | | |
| None | — | — | 67 | 14.1 |
| None | — | — | 67 | 13.8 |
| Dimethyl isosorbide | 0.871 | 5 | 66 | 13.4 |
| 15-Crown-5 | 1.106 | 5 | 66 | 11.8 |
| Boric acid | 0.301 | 5 | 64 | 13.1 |
| Piracetam | 0.711 | 5 | 58 | 11.6 |
| Benzyl acetoacetate | 0.966 | 5 | 53 | 11.5 |
| Phenylboronic acid | 0.591 | 5 | 52 | — |
| trans-4-hydroxy- | 0.655 | 5 | 49 | 12.3 |
| L-proline | | | | |
| Kojic acid | 0.053 | 0.4 | 48 | 11.8 |
| dioxane | 0.444 | 5 | 48 | |
| N,N-Dimethyl-L- | 0.191 | 1 | 42 | 11.8 |
| phenylalanine | | | | |
| Gluconolactone | 0.893 | 5 | 36 | — |
| quinic acid | 0.902 | 5 | 31 | — |
| Tryptamine HCl | 0.397 | 2 | 29 | 12.5 |
| Trigonelline | 0.862 | 5 | 21 | — |
| Tetramethyl- | 0.589 | 5 | 7 | — |
| ethylenediamine | | | | |
| 1-methyl-1H- | 0.126 | 1 | — | 10.5 |
| imidazole-5- | | | | |
| carboxylic acid | | | | |

### Example 51: Thermal Degradation Assay with Infliximab

REMICADE® infliximab was obtained from the Clinigen Group and reconstituted according to the instructions in the Janssen package insert, resulting in a 10 mg/mL infliximab solution in 5 mM phosphate buffer, pH about 7, with 50 mg/mL sucrose and 0.05 mg/mL polysorbate 80. The reconstituted drug product was then combined 1:1 by volume with 50 mM sodium acetate buffer at pH 5. The resulting solution was then injected onto a small preparative scale cation exchange column (GE Healthcare, Chicago, Ill.). After the infliximab was loaded onto the column, the column was washed with 10 column volumes of 50 mM sodium acetate buffer, pH 5. The infliximab was then eluted from the column with five column volumes of a 250 mM sodium chloride, 50 mM sodium acetate buffer at pH 5. The eluted infliximab was then buffer-exchanged into 20 mM phosphate buffer at pH 7 using Amicon Ultra 15 (EMD Millipore, Billerica, Mass.) centrifugal concentrators with a 30 kDa molecular weight cut-off. Stock solutions of either 4 or 8 mg/mL infliximab in 20 mM phosphate buffer at pH 7 were then used in subsequent tests.

All excipients used in this Example, listed in Table 52 below, were of the highest purity, and were obtained from Sigma Aldrich (St. Louis, Mo.) or Cayman Chemical (Ann Arbor, Mich.). The stock infliximab solutions were mixed with stock excipient solutions containing the excipients listed in Table 52 formulated in 20 mM phosphate buffer, pH 7 to achieve a final infliximab concentration of about 2 mg/mL in each sample. The samples containing the infliximab solutions and the stock excipient solutions were then aliquoted into five 100 μL aliquots in PCR tubes. The aliquots were incubated at 55° C. in a dry bath (Benchmark Scientific, Sayreville, N.J.) for different lengths of time, ranging from 15 minutes to about 3 hours to stress them thermally; the samples were then placed on ice once removed from the dry bath to quench thermal aggregation. These stressed samples were then analyzed for monomer content by high performance size exclusion chromatography (HP-SEC) using an Agilent 1100 series HPLC equipped with a diode array detector monitoring absorbance at 280 nm. The HPLC was operated at a column temperature of 25° C. with a mobile phase of 100 mM phosphate, 300 mM NaCl at pH 7 at a flow rate of 0.35 mL/min through a TSKgel SuperSW3000 4.6 mm×30 cm column (Tosoh Bioscience, Tokyo, Japan). For each sample, the monomer peak area was divided by the monomer peak area obtained from an identical but unstressed sample to obtain the percent monomer remaining after exposure to thermal stress. The remaining monomer as a percentage of the unstressed sample was then plotted as a function of incubation time and the absolute value of the slope of a linear fit to the data was recorded as the monomer loss rate. The determined monomer loss rate was then normalized by dividing the monomer loss rate by the monomer loss rate of the buffer control with no excipient, and the results are shown in Table 52 below.

TABLE 52

| | | |
|---|---|---|
| Excipient | Monomer | Normalized |

| | Conc | loss rate | monomer |
|---|---|---|---|
| ID | (mM) | (% mon/min) | loss rate |
| None | 0 | 0.1602 | 1 |
| Trehalose | 250 | 0.0781 | 0.488 |
| NaCl | 150 | 0.0727 | 0.454 |
| Arg HCl | 125 | 0.1186 | 0.740 |
| Etidronate | 100 | 0.0284 | 0.177 |
| Etidronate | 25 | 0.0824 | 0.514 |
| Etidronate | 50 | 0.0685 | 0.428 |
| Etidronate | 100 | 0.0321 | 0.200 |
| Trehalose | 200 | 0.0737 | 0.460 |
| Sorbitol | 200 | 0.0656 | 0.409 |

### Example 52: DLS Viscosity Measurements of Concentrated Omalizumab with Nicotinamide Mononucleotide and Itaconic Acid

Nicotinamide mononucleotide (NMN) was collected from nutritional supplement capsules purchased from Genex Formulas (Orlando, Fla.) and itaconic acid was purchased from Sigma-Aldrich (St. Louis, Mo.). These substances were used as excipients in the following experiment.

A biosimilar of the monoclonal antibody omalizumab acquired from Bioceros (The Netherlands) was buffer-exchanged into a 20 mM His HCl pH 6 buffer, and concentrated using an Amicon Ultra 15 centrifugal concentrator tube with a 30 kDa molecular weight cut-off (EMD Millipore, Billerica, Mass.). The buffer was prepared by dissolving 1.55 g of Histidine in 0.5 L Milli-Q water and adjusting the pH to 6.0 using 1 M HCl. The resulting concentrated formulation was analyzed by A280 for protein concentration by making serial dilutions of the concentrated formulation in 20 mM His HCl, loading 100 microliters of each dilution into a UV clear 96 half-well microplate (Greiner Bio-One, Austria), and measuring absorbance at a wavelength of 280 nm with a Synergy HT plate reader (BioTek, Winooski, Vt.). The blanked, path-length-corrected A280 measurement for each sample was then divided by the respective extinction coefficient and multiplied by the dilution factor to determine the protein concentration. Stock excipient solutions were prepared in 20 mM HisHCl pH 6 using the excipients mentioned above at 1 M or the solubility limit of the compound, and pH adjusted to 6 as necessary with either concentrated hydrochloric acid or sodium hydroxide. The concentrated protein formulation was then combined with the stock excipient solution or a control at a ratio of 9 parts protein formulation: 1 part excipient solution or buffer (for the control), and aliquots were added to the wells of a 384 well microplate (Aurora Microplates, Whitefish, Mont.). The microplate was then centrifuged at 400×g in a Sorvall Legend RT and shaken on a plate shaker. After the microplate was shaken, 2 microliters of a 5-fold dilution of polyethylene glycol surface-modified gold nanoparticles having a diameter of 100 nm (nanoComposix, San Diego, Calif.) in 20 mM His HCl were added to each sample well. The microplate was shaken a second time to mix the gold nanoparticles into the samples, and then it was placed in a DynaPro II DLS plate reader (Wyatt Technology Corp., Goleta, Calif.) to measure the apparent particle size of the gold nanoparticles at 25° C. The ratio of the apparent particle size of the gold nanoparticle in a protein formulation to the apparent particle size of the gold nanoparticle in buffer (no protein) was used to determine the viscosity of the protein formulation according to the Stokes-Einstein equation. In this example, the ratio of apparent radius to the actual radius of the gold nanoparticle was multiplied by the viscosity of water at 25° C. to calculate the viscosity of the protein formulation in centipoise (cP). Results using two different excipients are shown in Table 53 below.

TABLE 53

DLS Viscosity (cP)

| Excipient ID | Replicate 1 | Replicate 2 |
|---|---|---|
| Nicotinamide mononucleotide | 33.6 | 22.2 |
| Itaconic acid | 32.3 | 33.7 |
| No excipient (buffer control) | 73.6 | 78.4 |

### Example 53: DLS Viscosity Measurements of Concentrated Omalizumab

4-(2-Hydroxyethyl)piperazine-1-ethanesulfonic acid (HEPES), dicyclomine hydrochloride, pridinol methanesulfonate, 1-butylimidazole, and 1-hexylimidazole were purchased from Sigma Aldrich (St. Louis, Mo.) and used in preparing excipient solutions in this example. O-(octylphosphoryl)choline was purchased from Sigma Aldrich (St. Louis, Mo.) as a 1 M solution and used as a stock excipient solution in this example.

A concentrated formulation of a biosimilar of the monoclonal antibody omalizumab acquired from Bioceros (The Netherlands) was prepared as described in Example 52, and it was analyzed for protein concentration as described in Example 52. Stock excipient solutions using the excipients mentioned above were prepared in 20 mM HisHCl pH 6 buffer (prepared as described in Example 52) at 1 M or the solubility limit of the compound, and pH adjusted to 6 as necessary with either concentrated hydrochloric acid or sodium hydroxide. O-(octylphosphoryl)choline was used as a stock excipient without additional preparation. The concentrated protein formulation was then combined with a stock excipient solution or a control at a ratio of 9 parts protein formulation; 1 part excipient solution or buffer (for the control), and aliquots were added to the wells of a 384 well microplate (Aurora Microplates, Whitefish, Mont.). The microplate was then centrifuged at 400×g in a Sorvall Legend RT and shaken on a plate shaker. After the microplate was shaken, 2 microliters of a 5-fold dilution of polyethylene glycol surface-modified gold nanoparticles having a diameter of 100 nm (nanoComposix, San Diego, Calif.) in 20 mM His HCl were added to each sample well. The microplate was shaken a second time to mix the gold nanoparticles into the samples, and then it was placed in a DynaPro II DLS plate reader (Wyatt Technology Corp., Goleta, Calif.) to measure the apparent particle size of the gold nanoparticles at 25° C. The ratio of the apparent particle size of the gold nanoparticle in a protein formulation to the apparent particle size of the gold nanoparticle in buffer (no protein) was used to determine the viscosity of the protein formulation according to the Stokes-Einstein equation. In this example, the ratio of apparent radius to the actual radius of the gold nanoparticle was multiplied by the viscosity of water at 25° C. to calculate the viscosity of the protein formulation in centipoise (cP). Results using five different excipients are shown in Table 54 below.

TABLE 54

DLS Viscosity (cP)

| Excipient ID | Replicate 1 | Replicate 2 |
|---|---|---|
| HEPES | 15.9 | 17.4 |
| Dicyclomine HCl | * | * |
| Pridinol Methanesulfonate | 45.5 | 36.2 |
| O-(octylphosphoryl)choline | 35.6 | 35.4 |
| 1-butylimidazole | 16.8 | 15.6 |
| 1-hexylimidazole | 13.6 | 14.0 |
| None (buffer control) | 63.1 | 52.1 |

* Combination of excipient with protein resulted in reversible precipitation

### Example 54: DLS Viscosity Measurements of Concentrated Omalizumab

The following chemicals were used to prepare stock excipient solutions for this example: tetraethylammonium chloride, tetramethylammonium acetate, 1-methylimidazole, 1-butylimidazole, 1-hexylimidazole, 2-ethylimidazole, 2-methylimidazole, and spectinomycin were purchased from Sigma Aldrich (St. Louis, Mo.). Triglycine, tetraglycine, and 2-butylimidazole were purchased from Chem-Impex (Wood Dale, Ill.). Hordenine HCl was purchased from Bulk Supplements (Henderson, Nev.).

A concentrated formulation of a biosimilar of the monoclonal antibody omalizumab acquired from Bioceros (The Netherlands) was prepared as described in Example 52, and it was analyzed for protein concentration as described in Example 52. Stock excipient solutions using the excipients mentioned above were prepared in 20 mM HisHCl pH 6 buffer (prepared as described in Example 52) at 1 M or the solubility limit of the compound, and pH adjusted to 6 as necessary with either concentrated hydrochloric acid or sodium hydroxide. The concentrated protein formulation was then combined with a stock excipient solution or a control at a ratio of 9 parts protein: 1 part excipient solution or buffer (for the control), and aliquots were added to the wells of a 384 well microplate (Aurora Microplates, Whitefish, Mont.). The microplate was then centrifuged at 400×g in a Sorvall Legend RT and shaken on a plate shaker. After the microplate was shaken, 2 microliters of a 5-fold dilution of polyethylene glycol surface-modified gold nanoparticles having a diameter of 100 nm (nanoComposix, San Diego, Calif.) in 20 mM His HCl were added to each sample well. The microplate was shaken a second time to mix the gold nanoparticles into the samples, and then it was placed in a DynaPro II DLS plate reader (Wyatt Technology Corp., Goleta, Calif.) to measure the apparent particle size of the gold nanoparticles at 25° C. The ratio of the apparent particle size of the gold nanoparticle in a protein formulation to the apparent particle size of the gold nanoparticle in buffer (no protein) was used to determine the viscosity of the protein formulation according to the Stokes-Einstein equation. In this example, the ratio of apparent radius to the actual radius of the gold nanoparticle was multiplied by the viscosity of water at 25° C. to calculate the viscosity of the protein formulation in centipoise (cP). Results using 12 different excipients are shown in Table 55 below.

TABLE 55

| | DLS Viscosity (cP) | |
|---|---|---|
| Excipient ID | Replicate 1 | Replicate 2 |
| Tetraethylammonium HCl | 73.7 | 58.3 |
| Tetramethylammonium acetate | 68.2 | 85.7 |
| 1-methylimidazole | 53.4 | 54.8 |
| 1-butylimidazole | 40.5 | 74.0 |
| 1-hexylimidazole | 27.0 | 33.1 |
| 2-ethylimidazole | 57.5 | 71.8 |
| 2-butylimidazole | 26.0 | — |
| 2-methylimidazole | 65.6 | 81.3 |
| Triglycine | 70.0 | 79.5 |
| Tetraglycine | 89.7 | 72.2 |
| Hordenine HCl | 36.9 | 22.8 |
| Spectinomycin | 60.1 | 33.9 |
| None (buffer control) | 121.2 | 121.6 |

### Example 55: Excipients Increasing Thermal Stability

Therapeutic proteins are frequently subjected to fluctuations in temperatures, which may lead to changes in their tertiary and secondary structural elements. This leads to aggregation of the protein and a decrease in the active native species. Excipients protecting against thermal stress were tested by thermal degradation studies in the presence or absence of the excipients. The excipient stock was prepared by dissolving the excipients listed in Table 56 below at a concentration of 100 mg/mL in 20 mM Histidine buffer, pH 6.0 (prepared as described in Example 52). Each test sample was prepared by adding the excipient stock to the buffer to attain a final concentration of 5 mg/mL of the excipient and diluting protein from the 20 mg/mL ustekinumab stock in histidine buffer (prepared as described in Example 51) to the final concentration of 1 mg/mL. The formulation was aliquoted into 0.2 mL microcentrifuge tubes and incubated at 65 deg C. in a heating block for 120 min. Aliquots were withdrawn at 0 min, 30 min, 60 min, 90 min and 120 min. The samples were then quenched on ice for 5 min and spun down at 9000 rpm for 10 min. Following this, samples of the supernatant were analyzed by SE-HPLC as follows: the supernatant was loaded onto an Agilent 1100 HPLC system fitted with TSKgel SW3000 size exclusion chromatography column (30 cm×4.8 mm ID) and Agilent G1351B Diode array detector monitoring at 280 nm. 0.5% Phosphoric Acid, 150 mM NaCl, pH 3.5 mobile phase was used at a flow rate of 0.35 mL/min. For each sample, the monomer fraction was calculated by integrating the peak areas under the monomer peak and changes in the integrated peak area plotted as a function of time. The thermal stability was correlated with the fraction of monomer remaining at the end of the 2 h incubation, and the increase in percent monomer compared with the control (without added excipient) was recorded. The results for the seven tested excipients are summarized in Table 56 below.

TABLE 56

| | | % increase in monomer |
|---|---|---|
| Excipient | Class | content vs. control |
| Altrose | carbohydrates | 2.8 |
| Turanose | carbohydrates | 5.8 |
| N-Acetyl Mannosamine | carbohydrates | 3.1 |
| Gulonolactone | carbohydrates | 2.6 |
| cellobiosan | carbohydrates | 2.65 |
| Kestose | carbohydrates | 5.9 |
| Sedoheptulose | carbohydrates | 4.1 |

### Example 56: Excipients Improving Thermal Stability of ADCs

Antibody-drug conjugates (ADCs) are therapeutic proteins that are generated via the conjugation of small molecules to monoclonal antibodies through a chemical linker that allows site-specific delivery of the small molecule drug. The conjugated linker and small molecule combination alters the chemical and physical nature (charge, hydrophobicity, etc.) of the ADC as compared to its protein precursor and introduces additional stability concerns. The compound ustekinumab-FITC of Example 59 was used as a model ADC compound for these tests. Excipients protecting the model ADC against thermal stress were tested by thermal degradation studies in the presence or absence of the excipients. The excipient stock was prepared by dissolving the excipients listed in Table 57 below at a concentration of 100 mg/mL in 20 mM Histidine buffer, pH 6.0 (prepared as described in Example 52). Each test sample was prepared by adding the excipient stock to the buffer to attain a final concentration of 5 mg/mL and ustekinumab-FITC (as described in Example 59 below) in the histidine buffer to final concentration of 1 mg/mL of ustekinumab-FITC. The formulation was aliquoted into 0.2 mL microcentrifuge tubes and incubated at 65° C. in a heating block for 120 min. The aliquots were withdrawn at 0 min, 30 min, 60 min, 90 min and 120 min. The samples were then quenched on ice for 5 min and spun down at 9000 rpm for 10 min. The samples were analyzed by SE-HPLC where the supernatant was loaded onto an Agilent 1100 HPLC system fitted with TSK gel SW3000 size exclusion chromatography column (30 cm×4.8 mm ID) and Agilent G1351B Diode array detector monitoring at 280 nm. 0.5% Phosphoric Acid, 150 mM NaCl, pH 3.5 mobile phase was used at a flow rate of 0.35 mL/min. The monomer fraction was calculated by integrating the peak areas under the monomer peak and changes in the integrated peak area plotted as a function of time, and the increase in percent monomer compared with the control (without added excipient) was recorded. The thermal stability was correlated with the fraction of monomer remaining at the end of the 2 h incubation.

TABLE 57

| | | % increase in monomer |
|---|---|---|
| Excipient | class | content vs. control |
| Turanose | Sugar | 48.06 |
| N-Acetyl | Sugar | 23.49 |
| Mannosamine | | |
| Sedoheptulose | Sugar | 37.68 |
| Gulonolactone | Sugar | 79.95 |
| Altrose | Sugar | 63.41 |
| Pullulan | Sugar | 63.18 |

### Example 57: Excipients Protecting Against Freeze/Thaw Stress

Therapeutic proteins are frequently kept at low temperatures to improve their kinetic stability and minimize structural perturbations that could lead to the aggregation of the protein and a decrease in the active native species. In certain cases, this might be done by freezing the formulation until use. However, the low temperatures, concentration gradients, and ice formation during repeated freezing and thawing can stress the protein. Excipients protecting against thermal stress were tested and identified by thermal degradation studies in the presence or absence of the excipients. The excipient stock was prepared by dissolving the excipients listed in Table 58 below at a concentration of 1M in 20 mM Histidine buffer, pH 6.0, prepared as described in Example 52. Each test sample was prepared by adding the excipient stock to a final concentration of 100 mM of the excipient and diluting protein from the 20 mg/mL omalizumab stock in histidine buffer (prepared as described in Example 50) to final concentration of 2 mg/mL; the control was prepared in the same way, but without adding the excipient stock. The formulations were then aliquoted into 0.5 mL cryovials and frozen at −80° C. for 120 min. The samples were then thawed using a water bath kept at room temperature. This freeze-thaw cycle was repeated 6 times, following which each sample was aliquoted into 0.2 mL microcentrifuge tubes and spun down at 9000 rpm for 10 min. The samples were analyzed by SE-HPLC where the supernatant was loaded onto an Agilent 1100 HPLC system fitted with TSK gel SW3000 size exclusion chromatography column (30 cm×4.8 mm ID) and Agilent G1351B Diode array detector monitoring at 280 nm. 0.5% Phosphoric Acid, 150 mM NaCl, pH 3.5 mobile phase was used at a flow rate of 0.35 mL/min. The monomer fraction was calculated by integrating the peak areas under the monomer peak and changes in the integrated peak area plotted as a function of time.

TABLE 58

| | | % increase in monomer |
|---|---|---|
| Excipient | class | content vs. control |
| Kestose | Sugar | 71.18 |
| Turanose | Sugar | 75.99 |
| N-acetyl | Sugar | 6.63 |
| mannosamine | | |
| Sedoheptulose | Sugar | 11.35 |
| Trehalose | Sugar | 77.68 |
| Gulonolactone | Sugar | 10.02 |

### Example 58: Accelerated Aging Study

Excipients protecting against thermal stress were tested by thermal degradation studies in the presence or absence of the excipients. The excipient stock was prepared by dissolving the excipients listed in Table 59 below at a concentration of 1 M in 20 mM Histidine buffer, pH 6.0 (prepared as described in Example 52). Each test sample was prepared by adding the excipient to a final concentration of 100 mM and diluting protein from the 20 mg/mL ustekinumab stock in histidine buffer to final concentration of 2 mg/mL; the control was prepared in the same way, but without adding the excipient stock. 1 mL of each sample was aliquoted into 2 mL glass vials (West Pharmaceutical services, PA) and incubated at 40° C. for 4 weeks. The aliquots were withdrawn after 0, 1, 2, 3, and 4 weeks. The samples were analyzed by SE-HPLC where the supernatant was loaded onto an Agilent 1100 HPLC system fitted with TSKgel SW3000 size exclusion chromatography column (30 cm×4.8 mm ID) and Agilent G1351B Diode array detector monitoring at 280 nm. 0.5% Phosphoric Acid, 150 mM NaCl, pH 3.5 mobile phase was used at a flow rate of 0.35 mL/min. The monomer fraction was calculated by integrating the peak areas under the monomer peak and changes in the integrated peak area plotted as a function of time. The thermal stability was correlated with the fraction of monomer remaining at the end of the 4 week incubation.

TABLE 59

| | | % increase in monomer |
|---|---|---|
| Excipient | Class | content vs. control |
| Sedoheptulose | carbohydrates | 7.7 |
| Turanose | carbohydrates | 8.3 |
| N-Acetyl | carbohydrates | 4.4 |
| Mannosamine | | |
| Kestose | carbohydrates | 8.4 |

### Example 59: Synthesis of Ustekinumab FITC

A model compound to represent an antibody drug conjugate (ADC) was synthesized as follows. Ustekinumab was purchased from Bioceros (Utrecht, The Netherlands) as frozen aliquots at mAb concentration of 26 mg/mL in an aqueous 40 mM sodium acetate, 50 mM tris-HCl buffer at pH 5.5. The sample was buffer exchanged into a carbonate buffer at pH 9.2 and then incubated with 5 equivalents of fluorescein isothiocyanate (FITC) dissolved in anhydrous dimethyl sulfoxide, resulting in incorporation of 1.6 equivalents of FITC per equivalent of ustekinumab. The average mole ratio of FITC to ustekinumab was determined by measuring absorbance at 280 nm (representing protein+FITC) and absorbance at 495 nm (representing FITC). The calculations used 1.61 L/g·cm as the extinction coefficient of the mAb at 280 nm, 148,600 as the MW of the mAb, and 68,000 L/g·cm as the extinction coefficient for FITC at 495 nm. Excess unreacted FITC was removed by dialysis with 20 mM histidine buffer at pH 6. Next, the sample was concentrated to 15 mg/mL using an Amicon 30 kDa MWCO centrifuge tube.

### Example 60: Stability of Ustekinumab-FITC

The ADC model compound of Example 59 was diluted in 20 mM histidine buffer at pH 6 (prepared as described in Example 52) to a mAb concentration of 1 mg/mL. Samples were prepared with the added excipients listed in Table 60 below, and tested for their ability to protect the model ADC compound from mechanical shear stress. The samples were mechanically stressed by placing on a shaker table at 300 rpm for 72 h at 23° C. After the solutions were stressed, the particle size of the ADC complex was determined by dynamic light scattering. The control sample after shear had a particle radius of 143 nm, indicating significant aggregation compared with an unstressed sample (radius 5.5 nm). The excipient-containing samples did not show a significant increase in particle radius compared with the unstressed control sample (radius 5.5 nm), demonstrating a protective effect against mechanical shear stresses. Results are shown in Table 60 below.

TABLE 60

| | Excipient Concentration | DLS particle radius |
|---|---|---|
| Excipient | (mg/mL) | (nm) after shear test |
| None | 0 | 143 |
| Tubeimoside | 2.5 | 5.4 |
| Rebaudioside A | 5 | 5.8 |
| PPG1000 | 1 | 6.1 |
| PS80 | 1 | 5.8 |
| Rubusoside | 8.3 | 6.2 |
| Madecassoside | 5 | 6.4 |

### Example 61: Impact of Co-Solute on Caffeine Solubility in Aqueous Buffer During Refrigerated Storage

A 25 mM histidine buffer, pH 6 was prepared by dissolving 0.387 g of histidine in Milli-Q Type 1 water, titrating to pH 6 with hydrochloric acid, and diluting to a final volume of 100 mL with Milli-Q water. The buffer was then used to prepare 50 mM co-solute solutions, using the following excipients: sodium benzoate, 1-methyl-2-pyrrolidone, proline, phenylalanine, arginine monohydrochloride, benzyl alcohol, and nicotinamide. Into a 5 mL aliquot of each resulting solution was dissolved about 0.1 g caffeine to achieve a caffeine concentration of 20 mg/mL. Different volumetric ratios of 20 mg/mL caffeine with co-solutes and the corresponding solution containing the excipient solutions but no caffeine were prepared in triplicate in a 96 well microplate, maintaining a total well volume of 300 μL in all cases. The resulting microplates were then sealed with microplate tape and stored in a refrigerator with the temperature maintained in the range of 2 to 5° C. Over the course of storage, the microplates were visually observed for evidence of precipitate in the well. The earliest observed precipitate of the three wells for each condition was recorded, and results are summarized in Table 61 below.

TABLE 61

| | Caffeine Conc. | | | | | |
|---|---|---|---|---|---|---|
| Co-solute | (mg/mL) | Day 1 | Day 7 | Day 18 | Day 27 | Day 56 |
| Sodium | 7.5 | — | — | — | — | — |
| Benzoate | | | | | | |
| Sodium | 10 | — | — | — | — | — |
| Benzoate | | | | | | |
| Sodium | 12.5 | — | — | — | — | — |
| Benzoate | | | | | | |
| Sodium | 15 | — | — | — | — | PPT |
| Benzoate | | | | | | |
| Sodium | 20 | — | — | PPT | PPT | PPT |
| Benzoate | | | | | | |
| Benzyl alcohol | 7.5 | — | — | — | — | — |
| Benzyl alcohol | 10 | — | — | — | — | — |
| Benzyl alcohol | 12.5 | — | — | — | — | — |
| Benzyl alcohol | 15 | — | — | — | PPT | PPT |
| Benzyl alcohol | 20 | | PPT | PPT | PPT | PPT |
| 1-methyl-2- | 7.5 | — | — | — | — | — |
| pyrrolidone | | | | | | |
| 1-methyl-2- | 10 | — | — | — | — | — |
| pyrrolidone | | | | | | |
| 1-methyl-2- | 12.5 | | | | | PPT |
| pyrrolidone | | | | | | |
| 1-methyl-2- | 15 | — | — | PPT | PPT | PPT |
| pyrrolidone | | | | | | |
| 1-methyl-2- | 20 | PPT | PPT | PPT | PPT | PPT |
| pyrrolidone | | | | | | |
| Nicotinamide | 7.5 | — | — | — | — | — |
| Nicotinamide | 10 | — | — | — | — | — |
| Nicotinamide | 12.5 | — | — | — | — | — |
| Nicotinamide | 15 | — | — | — | — | PPT |
| Nicotinamide | 20 | — | — | PPT | PPT | PPT |
| Proline | 7.5 | — | — | — | — | — |
| Proline | 10 | — | — | — | — | — |
| Proline | 12.5 | — | — | — | PPT | PPT |
| Proline | 15 | — | PPT | PPT | PPT | PPT |
| Proline | 20 | — | PPT | PPT | PPT | PPT |
| Phenylalanine | 7.5 | — | — | — | — | — |
| Phenylalanine | 10 | — | — | — | — | — |
| Phenylalanine | 12.5 | — | — | — | — | — |
| Phenylalanine | 15 | — | — | — | — | PPT |
| Phenylalanine | 20 | — | PPT | PPT | PPT | PPT |
| Arginine HCl | 7.5 | — | — | — | — | — |
| Arginine HCl | 10 | — | — | — | — | PPT |
| Arginine HCl | 12.5 | — | — | — | PPT | PPT |
| Arginine HCl | 15 | — | PPT | PPT | PPT | PPT |
| Arginine HCl | 20 | PPT | PPT | PPT | PPT | PPT |
| None | 7.5 | — | — | — | — | — |
| None | 10 | — | — | — | — | PPT |
| None | 12.5 | — | — | — | PPT | PPT |
| None | 15 | — | — | PPT | PPT | PPT |
| None | 20 | — | PPT | PPT | PPT | PPT |

(PPT) = precipitate observed
(—) = clear solution observed

### Example 62: Testing Excipients for Reducing Viscosity of an Antibody Solution

A stock buffer solution of 20 mM histidine-HCl pH 6.0 (His HCl) was prepared by dissolving 1.55 g of histidine (Sigma-Aldrich, St. Louis, Mo.) in Type 1 ultrapure water. The contents were allowed to fully dissolve and the pH was adjusted to 6.0 using hydrochloric acid solution. After pH adjustment, the final volume was brought up to 0.5 L in a volumetric flask. All excipients were dissolved in His HCl and prepared at 10× concentration (1M) or at the solubility limit of the compound. Excipient solutions were pH measured and adjusted to pH 6.0 when needed.

In this example, protein solution with excipient concentration of 0.1M or lower was measured for viscosity. Biosimilar monoclonal antibody omalizumab purchased from Bioceros (The Netherlands) was buffer-exchanged into His HCl and concentrated to approximately 200 mg/mL using pre-rinsed Amicon-15 centrifugal devices (EMD Millipore, Billerica, Mass.) with a 30 kDa molecular weight cut-off limit. A dispersion of polyethylene glycol surface-modified gold nanoparticles (nanoComposix, San Diego, Calif.) was thoroughly mixed and diluted 5-fold into His HCl. In a separate PCR tube, 2.1 μL of gold-nanoparticles, 5.3 μL of 10× excipient solution and 47.6 μL of concentrated omalizumab was combined and thoroughly mixed. Each solution was transferred twice with a volume of 25 μL onto a 384-well plate (Aurora Microplates, Whitefish, Mont.) and centrifuged (Sorvall Legend RT) at 400×g for 1 minute. A tape seal was used to prevent evaporation of samples. The plate was then transferred to a DynaPro II DLS plate reader (Wyatt Technology Corp., Goleta, Calif.) to measure the apparent particle size of the gold nanoparticles at 25° C. The ratio of measured apparent radius to known radius particle size was calculated to determine the viscosity of protein formulation according to the Stokes-Einstein equation.

In this example, the diffusion interaction parameter (kD) of a dilute protein solution was measured by DLS in the presence of 0.1M or lower excipient. From the previously prepared excipient stock solutions, a 0.2M of excipient solution was prepared separately. The kD was measured using 5 different protein concentrations ranging from 10 mg/mL to 0.6 mg/mL in the presence of 0.1M excipient. 15 μL of protein solution was combined with 15 μL of 0.2M excipient solution (1:1 mixture) onto a 384-well plate (Aurora Microplates, Whitefish, Mont.). After loading the samples, the well plate was shaken on a plate shaker to mix the contents for 5 minutes. Upon mixing, the well plate was centrifuged at 400×g in a Sorvall Legend RT for 1 minute to force out any air pockets. The well plate was then loaded into a DynaPro II DLS plate reader (Wyatt Technologies Corp., Goleta, Calif.) and the diffusion coefficient of each sample was measured at 25° C. For each excipient, the measured diffusion coefficient was plotted as a function of protein concentration, and the slope of the linear fit of the data was recorded as the kD. The results are summarized in Table 62A and 62B below for two different series of tests.

TABLE 62A

| | Viscosity normalized | k_{D} |
|---|---|---|
| Excipient added | to control | (mh/g) |
| None (control) | 1.00 | −34.8 |
| 1,3-Dimethyl-2-imidazolidinone | 0.98 | −30.9 |
| 6-hydroxypyridine-2-carboxylic acid | 0.93 | −31.9 |
| Cyclohexane methylamine | 0.87 | −20.9 |
| 1,5-naphthalenedisulfonic acid | 0.83 | −25.1 |
| caffeic acid | 0.73 | −33.9 |
| Aspartame | 0.71 | ** |
| 1-Adamantyl-ethylamine HCl | 0.64 | −22.0 |
| 1,3-Diaminopropane | 0.49 | −11.3 |

**= data error, no k_{D} information is available for this test.

TABLE 62B

| | Viscosity normalized | k_{D} |
|---|---|---|
| Excipient Added | to control | (mL/g) |
| m-xylylenediamine | 0.22 | −13.1 |
| 1,3-diaminopropane | 0.22 | −11.4 |
| spermidine | 0.31 | −13.7 |
| nicotinic acid | 0.55 | −24.5 |
| ethanolamine HCl | 0.51 | −22.4 |
| lysine | 0.46 | −10.1 |
| 4-aminopyridine | 0.59 | −22.5 |
| quinic acid | 0.67 | −24.2 |
| folinic acid calcium salt | 0.93 | −34.9 |
| nicotinamide | 0.83 | −31.4 |
| mononucleotide | | |
| cysteamine HCl | 0.73 | −18.5 |
| None (control) | 1.00 | −30.0 |
| DL-3-phenylserine | 1.04 | −30.7 |
| dipyridamole | 1.10 | −31.2 |
| sarcosine | 1.33 | −29.9 |

### Example 63: Preparation of Protein Formulation Using Infliximab

Clinical grade REMICADE® (Janssen) was purchased from Pharmaceutical Buyers Inc. (New Hyde Park, N.Y.) and each vial contained lyophilized infliximab (100 mg) with Na₂HPO₄-2H₂O (6.1 mg), NaH₂PO₄—H₂O (2.2 mg), polysorbate 80 (0.5 mg), and sucrose (500 mg). This REMICADE® formulation was reconstituted to 10 mg/mL protein concentration with deionized (DI) water per the instruction of manufacturer. REMICADE-derived solutions containing 10 mg/mL of infliximab were then exchanged into formulation vehicles to produce test formulations with and without caffeine, as set forth in Table 63 below, using Amicon Ultra-15 centrifugal concentration units with filter membrane of 30 kDa MWCO. For this purpose, 15 mL of reconstituted infliximab solution was added to a centrifugal unit, and the solution volume was reduced to about 2 mL by centrifugation. Buffer exchange was then conducted three times with about 13 mL of formulation vehicle each time. After buffer exchange, about 5 mL of infliximab formulation was recovered from the centrifugal unit. The concentration of infliximab was determined using calibrated peak area by size exclusion chromatography (SEC) with a standard curve generated with infliximab at known concentrations. SEC analysis was done with an Agilent 1100 series HPLC equipped with a diode array detector monitoring absorbance at 280 nm. The HPLC was operated at a column temperature of 25° C. with a mobile phase of 100 mM phosphate, 300 mM NaCl at pH 7 at a flow rate of 0.35 mL/min through a TSKgel SuperSW3000 4.6 mm×30 cm column (Tosoh Bioscience, Tokyo, Japan). The final infliximab concentration was adjusted to 20 mg/mL using formulation vehicles, and the following two infliximab test formulations were prepared, as shown in Table 63, to be used for lyophilization studies as described below.

TABLE 63

| Test | Infliximab, | Sucrose, | Sodium | | Caffeine, |
|---|---|---|---|---|---|
| Formulation # | mg/mL | mg/mL | phosphate, mM | pH | mM |
| F1 | 20 | 13.4 | 5 | 7.2 | 0 |
| F2 | 20 | 13.4 | 5 | 7.2 | 6.7 |

### Example 64: Preparation of Protein Formulation Using Ipilimumab

Research grade ipilimumab (biosimilar of YERVOY®) was purchased from Bioceros Holding B.V. (Utrecht, NL) and the antibody had been purified by Protein A, viral inactivation, and anion exchange before use. A procedure similar to that of Example 63 was followed to exchange 20 mg/mL ipilimumab stock solution to produce liquid test formulations with and without caffeine, with the test formulations also containing mannitol, NaCl, polysorbate 80, and tris buffer as set forth in Table 64 below. Two ipilimumab test formulations were prepared, to be used for lyophilization studies as described below.

TABLE 64

| Test | Ipilimumab, | Mannitol, | NaCl, | PS80, | | | Caffeine, |
|---|---|---|---|---|---|---|---|
| Formulation # | mg/mL | mg/mL | mg/mL | mg/mL | Tris, mM | pH | mM |
| F3 | 30 | 10 | 1 | 0.02 | 5 | 7 | 0 |
| F4 | 30 | 10 | 1 | 0.02 | 5 | 7 | 6.7 |

### Example 65: Dissolution Time, Visual Inspection, and Viscosity Measurements for Test Formulations Reconstituted after Lyophilization

For test formulations F1 and F3 (prepared according to Examples 63 and 64), lyophilization was performed in quadruplicate, so that four lyophilized specimens were obtained for each of these two test formulations. For test formulations F2 and F4 (prepared according to Examples 63 and 64), lyophilization was performed in duplicate, and two lyophilized specimens were obtained for each of these two test formulations.

For each specimen to be lyophilized, 3 mL of liquid formulation was placed in a 10 mL glass vial. Each sample was then subjected to lyophilization with the cycle parameters summarized in Table 65a. Briefly, all the samples were first frozen to −45° C. and held for 2 hours. Primary drying was then conducted at −25° C. and at a reduced pressure of 100 mTorr for about 48 hours, followed by secondary drying at 40° C. and 100 mTorr for 10 hours. With this conservative cycle, elegant lyophilized cakes were generated for all the formulations.

TABLE 65a

| Steps | Temperature | Temperature Ramp Rate | Hold Time | Pressure |
|---|---|---|---|---|
| 1 | 5° C. | — | 0 min | Atmospheric |
| 2 | 0° C. | 1.0° C./min | 120 min | Atmospheric |
| 3 | −45° C. | 1.0° C./min | 120 min | Atmospheric |
| 4 | −45° C. | — | 32 min | 100 mTorr |
| 5 | −25° C. | 0.5° C./min | 2853 min | 100 mTorr |
| 6 | 40° C. | 0.2° C./min | 600 min | 100 mTorr |
| 7 | 15° C. | 1.0° C./min | NA | 100 mTorr |

For each test formulation, half of the vials were reconstituted within 48 hours after lyophilization for analysis, while reconstitution and analysis for the other half of the vials were conducted after they had been stored without reconstitution at 40° C. for 4 weeks. To reconstitute the lyophilized products, 390 μL of DI water or 50 mM caffeine solution in deionized (DI) water was added to each vial to generate highly concentrated infliximab and ipilimumab formulations. Reconstituted formulations that had been lyophilized with and without caffeine were analyzed to compare their viscosity and stability at two points in time after lyophilization: immediately after lyophilization and after 4 weeks of 40° C. storage. In addition, one sample of each formulation that had been lyophilized without caffeine (F1 for infliximab and F3 for ipilimumab) was reconstituted only with DI water, while the other sample was reconstituted with a solution of 50 mM caffeine in DI water. Concentrations of infliximab and ipilimumab in the reconstituted formulations were determined by calibrated SEC peak area.

Specimens from the two time points (TO and 4 weeks at 40° C.) were reconstituted as described above. The dissolution process of each formulation was visually monitored, and the time for lyophilisate to completely dissolve was recorded. Reconstituted solutions were visually inspected for color and clarity. As shown in Table 65b, caffeine reduced dissolution time for both infliximab and ipilimumab. By visual inspection, caffeine formulation of infliximab (F2-W) also generated a visually clearer reconstituted solution compared with the formulation lyophilized in the absence of caffeine (F1-W and F1-C).

TABLE 65b

| | | | | Dissolution | | |
|---|---|---|---|---|---|---|
| | | | | time | | |
| ID for | | | | (min) | | |
| initial | | | ID for | | After 4 | Visual |
| liquid | | Reconstitution | reconstituted | At | weeks at | inspection |
| formulation | Protein | solution | Formulation | T0 | 40° C. | of solution |
| F1 | infliximab | DI Water | F1-W | 4 | 18 | Light |
| | | | | | | yellow, hazy |
| | | 50 mM | F1-C | 3.5 | 7 | Light |
| | | caffeine in | | | | yellow, |
| | | DI water | | | | slightly hazy |
| F2 | infliximab | DI Water | F2-W | 2.5 | 2 | colorless |
| F3 | ipilimumab | DI Water | F3-W | 35 | 40 | Light yellow |
| | | 50 mM | F3-C | 11 | 12 | Light yellow |
| | | caffeine in | | | | |
| | | DI water | | | | |
| F4 | ipilimumab | DI Water | F4-W | 7.5 | 10 | Light yellow |

After reconstitution of each specimen, infliximab and ipilimumab concentrations were measured by SEC method and the viscosity of each sample was measured with a RheoSense microVisc viscometer. The instrument was equipped with an A05 chip and housed in a 20° C. temperature-controlled chamber for viscosity measurement. For each viscosity measurement, about 100 μL of the liquid sample was loaded into a clean test pipette and place onto the microVisc instrument. The sample was then analyzed at a shear rate of 250/sec. Four measurements were obtained for each sample and their averaged viscosity was recorded as shown in Table 65c. Caffeine, included either in the lyophilized formulation or in the reconstitution solution, significantly reduced viscosity of reconstituted infliximab and ipilimumab formulations, with >8000 viscosity reduction compared to the formulations without caffeine (F1-W and F3-W).

TABLE 65c

| | | | | Measured after 4 | |
|---|---|---|---|---|---|
| | | Measured at T0 | | weeks at 40° C. | |
| | | mAb | | mAb | |
| ID for | | concentration | | concentration | |
| Formulation | | by SEC | Viscosity | by SEC | Viscosity |
| reconstituted | Antibody | (mg/mL) | (cP) | (mg/mL) | (cP) |
| F1-W | Infliximab | 128 | 78.9 ± 0.5 | 127 | 228.4 ± 6.2 |
| F1-C | Infliximab | 117 | 18.1 ± 0.4 | 125 | 27.5 ± 0.1 |
| F2-W | Infliximab | 118 | 17.1 ± 0.2 | 124 | 17.8 ± 0.3 |
| F3-W | Ipilimumab | 164 | 101.3 ± 1.0 | 176 | 131.8 ± 2.8 |
| F3-C | Ipilimumab | 178 | 27.8 ± 0.7 | 176 | 22.9 ± 0.9 |
| F4-W | Ipilimumab | 185 | 24.3 ± 0.2 | 171 | 19.4 ± 0.8 |

### Example 66: Stability Measurements for Test Formulations Reconstituted after Lyophilization

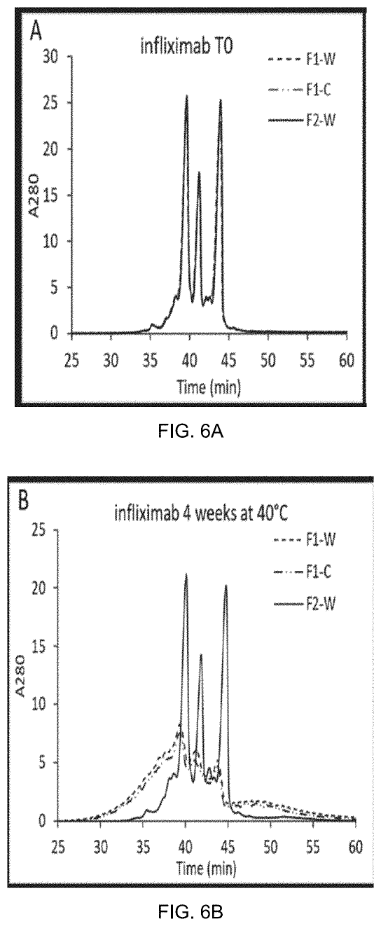

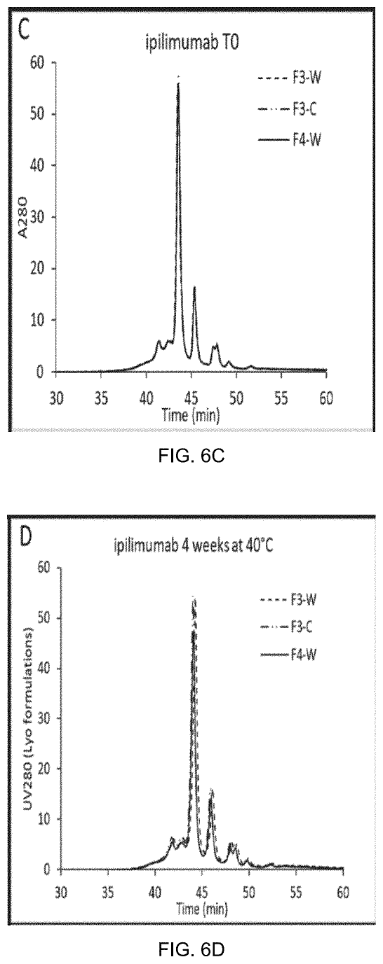

The effects of caffeine on the stability of infliximab and ipilimumab during lyophilization (T0) and after storage at 40° C. were studied. Changes in charge variants of the two antibodies during the freeze-drying process and storage were monitored by cation exchange chromatography (CEX-HPLC). For this purpose, reconstituted formulations were first diluted to about 5 mg/mL and loaded on to a Thermo Fisher ProPac WCX-10 cation exchange column of 4 mm in diameter and 25 cm in length. A salt gradient was applied to separate and elute different charge variants of the antibodies from the column while monitoring the UV signal at 280 nm. Results at TO showed that the presence of caffeine had no impact on the charge variants of either infliximab (as shown in FIG. 6A) or ipilimumab (as shown in FIG. 6C), with their CEX-HPLC chromatograms identical to the formulations lyophilized in the absence of caffeine. However, in the accelerated aging test samples (i.e., those samples reconstituted after 4 weeks at 40° C.), infliximab lyophilized without caffeine (F1-W and F1-C, as shown in FIG. 6B) showed significant changes in charge variants, indicating that the infliximab had experienced chemical degradation during the stability test. We concluded that infliximab lyophilized in the presence of caffeine (F2-W) was significantly more stable under the same thermal stress, showing little change in the distribution of charge variants after 4 weeks of storage. Lyophilized ipilimumab showed only minor changes in the distribution of charge variants in either of the formulations stored at 40° C. for 4 weeks (as shown in FIG. 6D).

### Example 67: Protein Aggregation Analyzed by Size Exclusion Chromatography

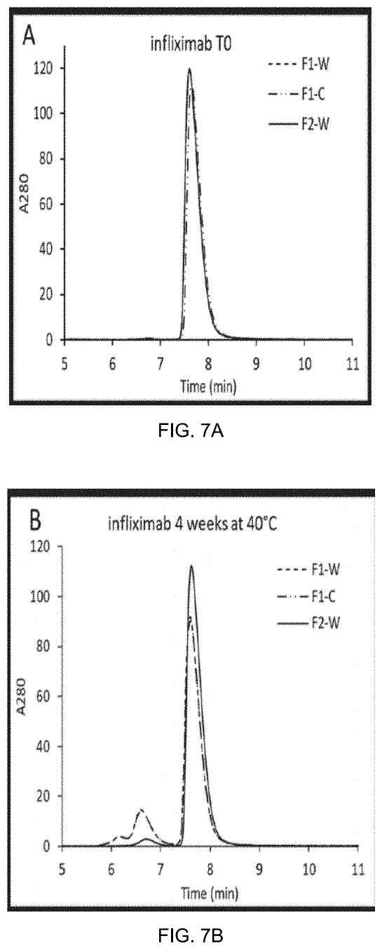

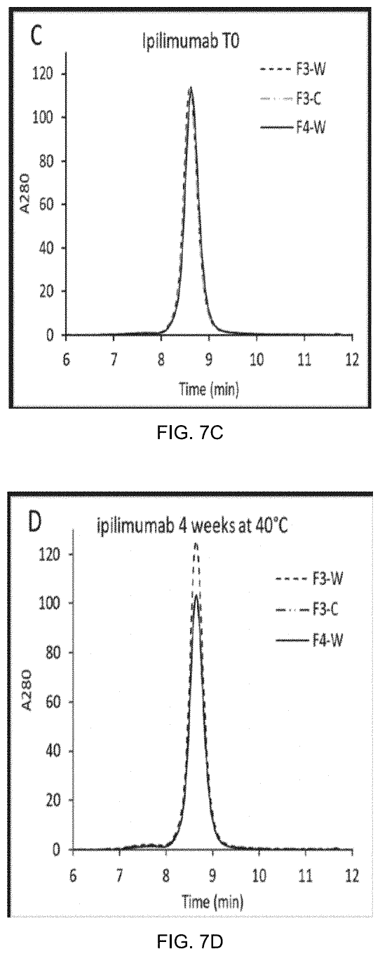

Size exclusion chromatography (SEC) was employed to analyze for the presence of soluble aggregates in the reconstituted formulations of the lyophilized antibodies prepared as described in Examples 64 and 65. For this purpose, reconstituted formulations of infliximab and ipilimumab were first diluted to about 2 mg/mL and loaded onto a Tosoh TSKgel Super SW3000 SEC column with a diameter of 4.6 mm and a length of 30 cm. An isocratic elution was applied to elute mAb species of different sizes from the column. UV signal at 280 nm was monitored. As shown in FIGS. 7A and 7C, reconstituted formulations for different lyophilisates (lyophilized with or without caffeine had the same SEC chromatograms. Furthermore, Table 66 summarizes SEC percent peak area of monomer and high molecular weight (HMW) species in the different formulations of the two antibodies. In the formulations reconstituted immediately after lyophilization, infliximab formulations (F1-W, F1-C and F2-W) contained about 98.7% of monomer and 1.3% of HMW species. Considering a 2% assay variation for this assay, there was no significant change in infliximab size distribution compared to the liquid formulations prior to lyophilization (pre-lyo), with 99.7% of monomer and 0.3% HMW species. Same results were observed for ipilimumab reconstituted immediately after lyophilization. The data suggest that caffeine does not affect physical stability of either infliximab or ipilimumab during the lyophilization process. For ipilimumab lyophilisates stored at 40° C. (F3-W, F3-C and F4-W), after 4 weeks of storage, there was a ˜1.5% decrease in monomer with a concomitant increase in HMW species, either with or without added caffeine. For infliximab lyophilized without caffeine (F1-W and F1-C), the stability was not good; the monomer decreased by about 17% from 98.7% to 81.7% (as shown in Table 66) with an increase of 17% in HMW species. The change in monomer and HMW species of infliximab lyophilized with caffeine was only 2% after the 4-week storage at 40° C. Caffeine not only significantly reduced viscosity of high concentration infliximab formulation, but also increased chemical and physical stability of lyophilized infliximab under thermal stress.

TABLE 66

| | Percent Monomer | | | Percent HMW | | |
|---|---|---|---|---|---|---|
| | pre-lyo | | After 4 | pre-lyo | | After 4 |
| Formulation | liquid | Lyophilized | weeks at | liquid | Lyophilized | weeks at |
| ID | formulation | T0 | 40° C. | formulation | T0 | 40° C. |
| F1-W | 99.7% | 98.7% | 81.6% | 0.3% | 1.3% | 18.4% |
| F1-C | | 98.7% | 81.7% | | 1.3% | 18.3% |
| F2-W | 99.7% | 99.2% | 97.1% | 0.3% | 0.9% | 2.9% |
| F3-W | 98.7% | 98.4% | 96.8% | 1.3% | 1.6% | 3.2% |
| F3-C | | 98.5% | 96.8% | | 1.5% | 3.2% |
| F4-W | 98.6% | 98.5% | 97.0% | 1.4% | 1.5% | 3.1% |

### Example 68: Sub-Visible Particles Measured by FlowCAM

Sub-visible particles in the formulations were analyzed using FlowCam VS1 instrument by Fluid Imaging Technologies. The instrument was equipped with a FC100 flow cell and a 10× objective. To analyze a formulation, the sample was diluted by 4 times using filtered formulation buffer. Analysis was conducted with 300 μL of sample and a flow rate of 0.15 mL/min. Duplicate samples were analyzed for each formulation. The concentration of particles in different size ranges was calculated by VisualSpreadsheet software.

FlowCam data for subvisible particle counts in different size ranges are listed in Table 67a for infliximab and Table 67b for ipilimumab. For infliximab formulation lyophilized with caffeine (F2-W), the formulations reconstituted at either TO or after 40° C. storage had lower total particle counts than the formulation lyophilized without caffeine (F1-W and F1-C), suggesting of caffeine could reduce aggregation of lyophilized infliximab. In case of ipilimumab, the total particle counts were significantly higher in reconstituted formulations for both time points (TO and 4 weeks at 40° C.). The data suggest that aggregation of ipilimumab could happen during reconstitution process, since caffeine either in the lyophilisate or reconstitution solution inhibited the aggregation.

TABLE 67a

| | | Particles per milliliter in | | | | |
|---|---|---|---|---|---|---|
| | | each particle size range | | | | |
| Formulation | Storage | | 2-10 | 10-20 | 20-50 | >50 |
| ID | conditions | Total | μm | μm | μm | μm |
| F1-W | | 71812 | 60348 | 8504 | 2196 | 768 |
| F1C | | | | | | |
| F2-W | Pre-lyo | 1536 | 1536 | 0 | 0 | 0 |
| | liquid | | | | | |
| | formulation | | | | | |
| F1-W | Lyophilized | 23244 | 20392 | 2304 | 548 | 0 |
| F1C | T0 | 28046 | 25362 | 2056 | 632 | 0 |
| F2-W | | 9078 | 7536 | 962 | 550 | 28 |
| F1-W | 4 weeks at | 36692 | 33922 | 2468 | 274 | 28 |
| F1C | 40° C. | 21768 | 19822 | 1564 | 356 | 28 |
| F2-W | | 14392 | 11760 | 2028 | 576 | 28 |

TABLE 67b

| | | Particles per milliliter in | | | | |
|---|---|---|---|---|---|---|
| | | each particle size range | | | | |
| Formulation | Storage | | 2-10 | 10-20 | 20-50 | >50 |
| ID | conditions | Total | μm | μm | μm | μm |
| F3-W | Pre-lyo | 6541 | 5856 | 508 | 164 | 14 |
| F3-C | liquid | | | | | |
| F4-W | formulation | 6527 | 5979 | 507 | 41 | 0 |
| F3-W | Lyophilized | 563308 | 492598 | 59470 | 10962 | 276 |
| F3-C | T0 | 179404 | 157698 | 17794 | 3568 | 362 |
| F4-W | | 152596 | 133926 | 13594 | 4698 | 440 |
| F3-W | After 4 | 1245242 | 1120360 | 113126 | 11536 | 222 |
| F3-C | weeks at | 336420 | 309598 | 24304 | 2502 | 0 |
| F4-W | 40° C. | 138254 | 126084 | 10468 | 1702 | 0 |

### Example 69: Lyophilization of Infliximab with Caffeine

Infliximab (Remicade®) was reconstituted with DI water per the instructions of manufacturer. In this Example, all infliximab solutions were prepared by reconstituting Remicade®) as described above. Infliximab solutions at 10 mg/mL were then exchanged into formulation vehicles containing four different levels of caffeine addition to other excipients as set forth in Table 68, using Amicon Ultra-15 centrifugal filter units with filter membrane of 30 kDa MWCO. The reconstituted samples were designated RF-INFL1 (0 mM caffeine), RF-INFL2 (50 mM caffeine), RF-INFL3 (25 mM caffeine), and RF-INFL4 (75 mM caffeine). To prepare the reconstituted samples to be used for lyophilization, 15 mL of reconstituted infliximab was added to a centrifugal filter unit and the volume was reduced to about 2 mL by centrifugation. Buffer exchange was then conducted three times with about 13 mL of formulation vehicle each time. Centrifugation was conducted at 3,200×g and room temperature. After buffer exchange, about 5 mL of Infliximab formulation was recovered from the centrifugal unit. The concentration of Infliximab was determined using peak area by SEC and a calibration curve generated with reconstituted infliximab diluted to different concentrations as the standards. Final infliximab concentration was adjusted to 20 mg/mL using the corresponding formulation vehicles. In each 3 mL glass vial, 0.8 mL of the final formulation was filled and subjected to lyophilization. A lyophilization cycle of about 48 hours was used, with the primary drying being conducted at −25° C. and secondary drying at 40° C.; details of the process are shown in Table 69. After lyophilization, one vial from each formulation was reconstituted immediately with DI water and analyzed. The rest of the vials were stored at 2-8° C. for 37 weeks or 40° C. for 12 weeks to monitor storage stability of lyophilized infliximab with and without caffeine. To generate highly concentrated infliximab formulations with a target concentration of 150 mg/mL, all of the vials were reconstituted to about 0.1 mL with DI water and the infliximab concentration in reconstituted formulations were measured by SEC method. Formulation compositions before lyophilization and after reconstitution are summarized in Table 68. The concentration change of PS80 from Remicade® was not monitored during the process; the PS80 concentration values in Table 68 were calculated based on volume changes of the formulations and an initial PS80 concentration of 0.05 mg/mL in 10 mg/mL reconstituted Remicade®. All four formulations with different concentrations of caffeine generated pharmaceutically elegant cakes.

TABLE 68

| | Pre-lyophilization | Reconstituted |
|---|---|---|
| Component | liquid formulation | formulation |
| infliximab (mg/mL) | 20 | 150 |
| sucrose (mg/mL) | 13.4 | 100.5 |
| PS80 (mg/mL) | 0.1 | 0.75 |
| caffeine (mM) | 0, 3.3, 6.7 or 10 mM | 0, 25, 50 and 75 mM |
| sodium phosphate | 5 mM | 37.5 mM |
| (mM) | | |

| | |
|---|---|
| pH | 7.2 |

TABLE 69

| Lyophilization Step | Temp | Rate | Hold Time | Pressure |
|---|---|---|---|---|
| 1 | 5° C. | | 0 min | |
| 2 | 0° C. | 1.0° C./min | 120 min | |
| 3 | −45° C. | 1.0° C./min | 120 min | |
| 4 | −45° C. | | 28 min | 100 mTorr |
| 5 | −25° C. | 0.5° C./min | 1595 min | 100 mTorr |
| 6 | 40° C. | 0.2° C./min | 600 min | 100 mTorr |
| 7 | 15° C. | 1.0° C./min | NA | 100 mTorr |

Dissolution time and appearance of the lyophilized samples were assessed according to the stability study plan shown in Table 70. The stability study was conducted at 40° C. for 12 weeks for all four formulations. In addition, a study of about 9 months (37 weeks) at 2-8° C. was performed with the formulation containing 50 mM caffeine in the reconstituted formulation (RF-INFL2), in comparison with the control formulation without caffeine (RF-INFL1).

TABLE 70

| | caffeine in | | | | | | | |
|---|---|---|---|---|---|---|---|---|
| | formulation | | 40° C. | | | 2-8° C. | | |
| Formulation | reconstituted | | 2 | 4 | 12 | 4 | 12 | 37 |
| ID | (mM) | T0 | weeks | weeks | weeks | weeks | weeks | weeks |
| RF-INFL1 | 0 | x | x | x | x | x | x | x |
| RF-INFL2 | 50 | x | x | x | x | x | x | x |
| RF-INFL3 | 25 | x | | x | x | | | |
| RF-INFL4 | 75 | x | | x | x | | | |

At each time point, one vial of each formulation was reconstituted for analysis. All of the reconstituted formulations with highly concentrated infliximab were colorless clear solutions. Reconstitution time was fast, considering the high concentration of infliximab in the formulations (Table 71). For the formulations containing caffeine, the reconstitution time was shorter than the control (RF-INFL1).

TABLE 71

| | Reconstitution time (minutes) | | | | | | |
|---|---|---|---|---|---|---|---|
| | for lyophilized samples in stability study | | | | | | |
| | | 40° C. | | | 2-8° C. | | |
| | | 2 | 4 | 12 | 4 | 12 | 37 |
| Formulation ID | T0 | weeks | weeks | weeks | weeks | weeks | weeks |
| RF-INFL1 | 3 | 4 | 5 | 5 | 4 | 4 | 7 |
| RF-INFL2 | 2 | 3 | 4 | 2 | 3 | 2 | 3 |
| RF-INFL3 | 4 | — | 3 | 3 | — | — | — |
| RF-INFL4 | 2 | — | 3 | 2 | — | — | — |

After reconstitution, infliximab concentrations were measured by SEC method and viscosity was measured with microVisc viscometer at 25° C., as summarized in Table 72. The reported viscosity is an average of four measurements. Viscosity reduction effect was seen for caffeine at all three levels with a concentration dependence plateaued at about 50 mM caffeine in the reconstituted formulation. Averaged viscosity reduction was 65%, 78% and 81% for caffeine concentration at 25 mM, 50 mM and 75 mM, respectively. This viscosity reducing effect maintained constant throughout the 37-week storage at 2-8° C.

TABLE 72

| Stability study time | | Formulation | Measured Infliximab | Viscosity |
|---|---|---|---|---|
| point | | ID | Concentration (mg/mL) | (cP) |
| T0 | | RF-INFL1 | 134 | 80.96 ± 1.60 |
| | | RF-INFL2 | 135 | 17.68 ± 1.08 |
| | | RF-INFL3 | 134 | 30.53 ± 0.56 |
| | | RF-INFL4 | 144 | 15.73 ± 0.15 |
| 40° C. | 2 weeks | RF-INFL1 | 130 | 91.34 ± 0.68 |
| | | RF-INFL2 | 134 | 16.99 ± 0.78 |
| | 4 weeks | RF-INFL1 | 137 | 88.13 ± 0.75 |
| | | RF-INFL2 | 141 | 21.85 ± 1.73 |
| | | RF-INFL3 | 132 | 29.56 ± 0.36 |
| | | RF-INFL4 | 137 | 20.00 ± 0.19 |
| | 12 weeks | RF-INFL1 | 144 | 98.12 ± 2.42 |
| | | RF-INFL2 | 135 | 18.42 ± 0.13 |
| | | RF-INFL3 | 135 | 34.52 ± 0.05 |
| | | RF-INFL4 | 132 | 15.51 ± 0.30 |
| 2-8° C. | 4 weeks | RF-INFL1 | 135 | 83.17 ± 0.89 |
| | | RF-INFL2 | 136 | 18.81 ± 0.11 |
| | 12 weeks | RF-INFL1 | 135 | 82.83 ± 4.34 |
| | | RF-INFL2 | 146 | 23.57 ± 1.36 |
| | 37 weeks | RF-INFL1 | 146 | 86.70 ± 0.78 |
| | | RF-INFL2 | 142 | 16.88 ± 0.02 |

Changes in charge variants of infliximab during the freeze-drying process and storage were monitored by CEX-HPLC. The chromatographic profiles for charge variants were the same before and after lyophilization, indicating that stability of infliximab during lyophilization and reconstitution processes was not affected by caffeine. Multiple charge variants were separated by IEX-HPLC. Changes in three main peaks, indicated as PK1, PK2 and PK3 at about 40, 41, and 45 minutes retention time, respectively. At 40° C., there was a slight decrease after 12 weeks of storage, which was about 700 for PK1, 300 for PK2 and 700 for PK3. All the lyophilized formulations were stable at 2-8° C. with no apparent change detected after 37 weeks of storage. Caffeine at three different concentrations showed no impact on the chemical stability of lyophilized infliximab during storage.

SE-HPLC was employed to analyze the soluble aggregates in the reconstituted formulations, showing that caffeine had no negative impact on the physical stability of infliximab during lyophilization process. Table 73 summarizes the monomer content and higher molecular weight species (HMWS) for liquid formulation before lyophilization, immediately after lyophilization (T0), as well as for samples stored at 2-8° C. or 40° C. At 2-8° C., all formulations essentially had no change by SEC, with the monomer peak content maintained at 9900 after 37 weeks. After 40° C. storage for 12 weeks, there was a decrease of about 50% in monomer peak for all formulations. Caffeine had no negative impact on the storage stability of lyophilized infliximab.

TABLE 73

| Time point | | Formulation ID | HMWS | Monomer |
|---|---|---|---|---|
| Pre-Lyophilization | | RF-INFL1 | 0.67% | 99.33% |
| | | RF-INFL2 | 0.76% | 99.24% |
| | | RF-INFL3 | 0.74% | 99.26% |
| | | RF-INFL4 | 0.61% | 99.39% |
| T0 | | RF-INFL1 | 1.15% | 98.85% |
| | | RF-INFL2 | 0.99% | 99.01% |
| | | RF-INFL3 | 1.03% | 98.98% |
| | | RF-INFL4 | 1.11% | 98.89% |
| 40° C. | 2 weeks | RF-INFL1 | 2.54% | 97.46% |
| | | RF-INFL2 | 2.18% | 97.81% |
| | 4 weeks | RF-INFL1 | 3.42% | 96.58% |
| | | RF-INFL2 | 3.26% | 96.74% |
| | | RF-INFL3 | 3.82% | 96.18% |
| | | RF-INFL4 | 4.11% | 95.89% |
| | 12 weeks | RF-INFL1 | 6.40% | 93.60% |
| | | RF-INFL2 | 5.44% | 94.56% |
| | | RF-INFL3 | 5.59% | 94.41% |
| | | RF-INFL4 | 5.49% | 94.51% |
| 2-8° C. | 4 weeks | RF-INFL1 | 0.86% | 99.14% |
| | | RF-INFL2 | 0.95% | 99.05% |
| | 12 weeks | RF-INFL1 | 0.94% | 99.06% |
| | | RF-INFL2 | 0.81% | 99.19% |
| | 37 weeks | RF-INFL1 | 0.80% | 99.20% |
| | | RF-INFL2 | 1.10% | 98.90% |

### EQUIVALENTS

While specific embodiments of the subject invention have been disclosed herein, the above specification is illustrative and not restrictive. While this invention has been particularly shown and described with references to preferred embodiments thereof, it will be understood by those skilled in the art that various changes in form and details may be made therein without departing from the scope of the invention encompassed by the appended claims. Many variations of the invention will become apparent to those of skilled art upon review of this specification. Unless otherwise indicated, all numbers expressing reaction conditions, quantities of ingredients, and so forth, as used in this specification and the claims are to be understood as being modified in all instances by the term “about.” Accordingly, unless indicated to the contrary, the numerical parameters set forth herein are approximations that can vary depending upon the desired properties sought to be obtained by the present invention.

1. A method of improving the stability of a reconstituted formulation of a therapeutic protein, comprising the steps of:
preparing a liquid formulation comprising the therapeutic protein;
lyophilizing the liquid formulation to produce a lyophilized formulation; and
reconstituting the lyophilized formulation with a reconstituting solvent to produce the reconstituted formulation,
the method further comprising adding an effective amount of a lyo-enhancing excipient to the liquid formulation before or during the lyophilizing step; and/or adding an effective amount of a lyo-enhancing excipient to the lyophilized formulation after the lyophilizing step;
wherein the reconstituted formulation has improved stability in comparison to the stability of a control formulation otherwise identical to the reconstituted formulation but lacking the lyo-enhancing excipient.

2. The method of claim 1, wherein the therapeutic protein is a therapeutic antibody.

3. The method of claim 2, wherein the therapeutic antibody is infliximab or ipilimumab.

4. The method of claim 1, wherein the lyo-enhancing excipient is a hindered amine.

5. The method of claim 4, wherein the hindered amine is caffeine or nicotinamide.

6. The method of claim 1, wherein the method comprises adding an effective amount of a lyo-enhancing excipient to the liquid formulation before or during the lyophilizing step.

7. The method of claim 6, wherein the method further comprises adding an effective amount of a second lyo-enhancing excipient to the lyophilized formulation.

8. (canceled)

9. (canceled)

10. The method of claim 1, wherein the method comprises adding an effective amount of a lyo-enhancing excipient to the lyophilized formulation.

11. The method of claim 10, wherein the lyo-enhancing excipient is added to the reconstituting solvent prior to the step of reconstituting the liquid formulation.

12. The method of claim 11, wherein the lyo-enhancing excipient is added to the reconstituting solvent in an amount such that the concentration of the lyo-enhancing excipient in the reconstituted formulation is between about 0.1 mg/mL and about 200 mg/mL.

13. (canceled)

14. (canceled)

15. The method of claim 11, wherein the lyo-enhancing excipient is added to the reconstituting solvent in an amount such that the mass concentration of the lyo-enhancing excipient in the reconstituted formulation is between about 1% and about 10% of the mass concentration of the therapeutic protein in the reconstituted formulation.

16-21. (canceled)

22. A method of improving the stability of a freeze concentrate formulation of a therapeutic protein, comprising the steps of:
preparing a liquid formulation comprising the therapeutic protein;
adding an effective amount of a lyo-enhancing excipient to the liquid formulation before the freezing step;
and freezing the liquid formulation to prepare a freeze concentrate formulation,
wherein the freeze concentrate formulation has improved stability in comparison to the stability of a control formulation otherwise identical to the freeze concentrate formulation but lacking the lyo-enhancing excipient.

23. A method of reducing the viscosity of a reconstituted formulation of a therapeutic protein, comprising the steps of:
preparing a liquid formulation comprising the therapeutic protein,
lyophilizing the liquid formulation to produce a lyophilized formulation, and reconstituting the lyophilized formulation with a reconstituting solvent to produce the reconstituted formulation,
wherein the method further comprises adding an effective amount of a lyo-enhancing excipient to the liquid formulation before or during the lyophilizing step, and/or wherein the method further comprises adding an effective amount of a lyo-enhancing excipient to the lyophilized formulation after the lyophilizing step,
wherein the viscosity of the reconstituted formulation is reduced in comparison to the stability of a control formulation otherwise identical to the reconstituted formulation but lacking the lyo-enhancing excipient.

24-45. (canceled)

46. A reduced viscosity formulation, wherein the formulation is a reconstituted formulation of a lyophilizate that comprises a therapeutic protein, the formulation further comprising:
a liquid medium, and a lyo-enhancing excipient,
wherein the therapeutic protein is present in the formulation in an amount of at least about 100 mg/ml;
wherein the lyo-enhancing excipient is caffeine or nicotinamide, wherein the lyo-enhancing excipient is present in the composition in an amount between about 2 and 50 mg/ml;
wherein the viscosity of the reduced viscosity formulation is at least about 30% lower than the viscosity of a control formulation otherwise identical to the reduced viscosity formulation but lacking the lyo-enhancing excipient.

47. The reduced viscosity formulation of claim 46, wherein the therapeutic protein is a therapeutic antibody.

48. The reduced viscosity formulation of claim 47, wherein the therapeutic antibody is infliximab or ipilimumab.

49. The reduced viscosity formulation of claim 46, wherein the lyo-enhancing excipient is caffeine.

50-76. (canceled)
